(19) United States
(12) Patent Application Publication
(10) Pub. No.: US 2026/0102357 A1
(43) Pub. Date: Apr. 16, 2026

(54) Sulfur-Containing Ionizable Lipids for the Delivery of Therapeutic Agents

(71) Applicant: NanoVation Therapeutics Inc., Vancouver (CA)

(72) Inventors: Deaglan Arnold, London (CA); Marco A. Ciufolini, Vancouver (CA); Fariba Saadati, Vancouver (CA)

(73) Assignee: NanoVation Therapeutics Inc., Vancouver, BC (CA)

(21) Appl. No.: 19/115,816

(22) PCT Filed: Sep. 27, 2023
(86) PCT No.: PCT/CA2023/051272

§ 371 Date: Mar. 27, 2025

Related U.S. Application Data
(60) Provisional application No. 63/410,281, filed on Sep. 27, 2022.

(51) Int. Cl. A61K 9/51; A61K 31/7088; C07C 323/25; C07C 323/52; C07D 307/12; C07D 307/14

(52) U.S. Cl. CPC A61K 9/5123; A61K 31/7088; C07C 323/25; C07C 323/52; C07D 307/12; C07D 307/14

(57) ABSTRACT

The present disclosure relates to a sulfur-containing ionizable lipid or a pharmaceutically acceptable salt thereof that incorporates a dithioacetal or dithioketal moiety in one or more of its lipophilic chains. Further provided is a delivery vehicle, such as a lipid nanoparticle, comprising the ionizable lipid for the delivery of cargo, such as nucleic acid.

### TECHNICAL FIELD

Provided herein are sulfur-containing lipids that may be formulated in a delivery vehicle so as to facilitate the encapsulation of cargo, such as, without limitation, nucleic acids (e.g., RNA or DNA), proteins, peptides, pharmaceutical drugs and salts thereof.

### BACKGROUND

Nucleic acid-based therapeutics have enormous potential in medicine. To realize this potential, however, the nucleic acid must be delivered to a target site in a patient. This presents challenges since nucleic acid is rapidly degraded by enzymes in the plasma upon administration.

Even if the nucleic acid is delivered to a disease site, there still remains the challenge of intracellular delivery. To address these problems, lipid nanoparticles have been developed that protect nucleic acid from such degradation and facilitate delivery across cellular membranes to gain access to the intracellular compartment, where the relevant translation machinery resides.

A key component of a lipid nanoparticle (LNP) is an ionizable lipid. The ionizable lipid is typically positively charged at low pH, which facilitates association with the negatively charged nucleic acid. However, the ionizable lipid is neutral at physiological pH, making it more biocompatible in biological systems. Further, it has been suggested that after the LNPs are taken up by a cell by endocytosis, the ionizability of these lipids at low pH enables endosomal escape. This in turn enables the nucleic acid to be released into the intracellular compartment.

An earlier example of an LNP product approved for clinical use and reliant on ionizable lipid is Onpattro®. Onpattro® is an LNP-based short interfering RNA (siRNA) drug for the treatment of polyneuropathies induced by hereditary transthyretin amyloidosis. Onpattro® is reliant on an ionizable lipid referred to as “DLin-MC3-DMA” or more commonly “MC3”, 1 (FIG. 1), by investigators. Furthermore, MC3 represents an evolution of a structurally related ionizable lipid, referred to by investigators as “KC2”, 2 (FIG. 1). MC3 is considered a state-of-the art ionizable lipid for the delivery of siRNA, requiring about 3 times less siRNA than KC2. Nonetheless, KC2 is superior in other applications and remains a valuable research tool.

While the foregoing ionizable lipids are especially efficacious for the delivery of siRNA-containing LNPs to hepatic cells, they are much less effective for the hepatic delivery of mRNA-containing LNPs. To illustrate, mRNA vaccines, including the COVID-19 Pfizer/BioNTech and Moderna vaccines, rely on lipid nanoparticles to deliver mRNA to the cytoplasm of liver cells. After entry into the host cell, the mRNA is transcribed to produce antigenic proteins. In the case of the COVID-19 vaccines, the mRNA encodes the highly immunogenic Sars-Cov-2 spike protein. Such vaccines, however, incorporate other types of ionizable lipids besides MC3 or KC2. In particular, the Pfizer/BioNTech vaccine comprises an ionizable lipid referred to as “ALC-0315”, 3 (Scheme 1), and the Moderna vaccine comprises an ionizable lipid referred to as “SM-102”, 4.

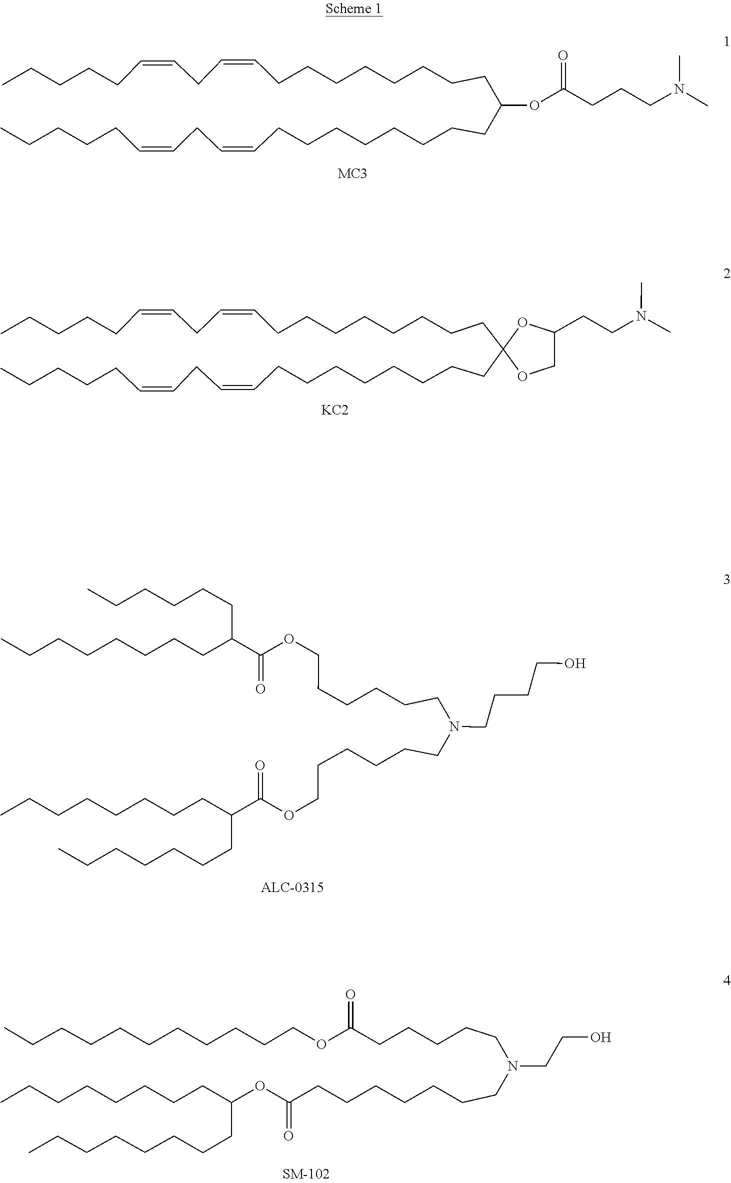

Furthermore, the above lipids were optimized for delivery of therapeutic nucleic acids to the liver. However, there remains a need to develop new lipids for the delivery of charged cargo, such as nucleic acids to other organs, such as the spleen, lungs, bone marrow, skin, etc. The delivery of cargo beyond the liver would expand the clinical utility of LNPs to target disease conditions that affect tissues and organs beyond the liver. There is also an ongoing need to develop LNPs with improved delivery of nucleic acid or other charged cargo to the liver.

The present disclosure seeks to address one or more of the above identified problems and/or provides useful alternatives to known products and/or compositions for the delivery of nucleic acid.

### Definitions

As used herein, “type 1 ionizable head” or “MC-type ionizable head” refers to a moiety that has a head group of the lipid of Formula I below, or equivalents thereof, with n¹ ranging from 1 to 5:

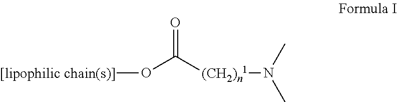

As used herein, “type 2 ionizable head” or “KC-type ionizable head” refers to a moiety that has a head group of the lipid of Formula II below, or equivalents thereof, with n² ranging from 1 to 5:

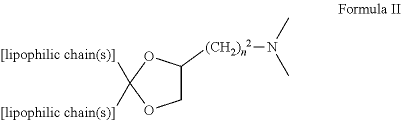

As used herein, “type 3 ionizable head” refers to a moiety that is the head group of the structure as defined by Formula III below, or equivalents thereof, with m¹ and n³ independently ranging from 1 to 5:

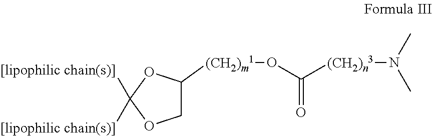

As used herein, “type 4 ionizable head” refers to a moiety that is the head group of the structure as defined by Formula IV below, or equivalents thereof, with m² and n⁴ independently ranging from 2 to 5:

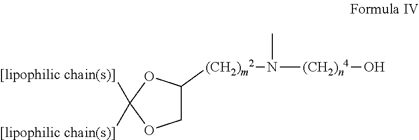

As used herein, “type 5 ionizable head” refers to a moiety that is the head group of the structure as defined by Formula V below, or equivalents thereof, with m³ and n⁵ independently ranging from 1 to 5:

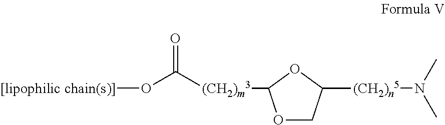

As used herein, “type 6 ionizable head” refers to a moiety that is the head group of the structure as defined by Formula VI below, or equivalents thereof, with m⁴ ranging from 1 to 5, and n⁶ independently ranging from 2 to 5:

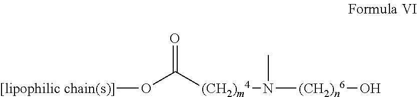

As used herein, “type 7 ionizable head” refers to a moiety that is the head group of the structure as defined by Formula VII below, or equivalents thereof, with n⁷ ranging from 1 to 5:

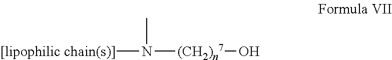

As used herein, “type 8 ionizable head” refers to a moiety that is the head group of the structure as defined by Formula VIII below, or equivalents thereof, with n⁸ ranging from 1 to 5:

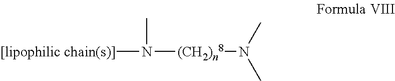

As used herein, “type 9 ionizable head” refers to a moiety that is the head group of the structure as defined by Formula IX below, or equivalents thereof, with m⁵ and n⁹ independently ranging from 1 to 5:

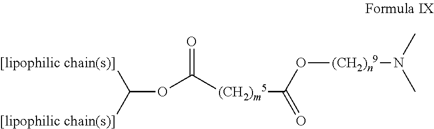

As used herein, the term “ionizable lipid” refers to a lipid that, at a given pH, is in an electrostatically neutral form and that may either accept or donate protons, thereby becoming electrostatically charged, and for which the electrostatically neutral form has a calculated logarithm of the partition coefficient between water and 1-octanol (i.e., a c Log P) that is greater than 8.

As used herein, the term “alkyl” or “alkyl group” as described herein is a carbon-containing chain that is linear or branched. The term is also meant to encompass a carbon-containing chain that optionally has varying degrees of unsaturation and that is optionally substituted.

As used herein, the term “Cₘ to Cₙ alkyl” or “Cₘ to Cₙ alkyl group” refers to a linear or branched carbon chain having a total minimum of m carbon atoms and up to n carbon atoms, and that is optionally unsaturated and optionally substituted. For example, a “C₁ to C₃ alkyl” or “C₁ to C₃ alkyl group” is an alkyl having between 1 and 3 carbon atoms.

The term “optionally substituted” with reference to an alkyl means that at least one hydrogen atom of the alkyl group can be replaced by a non-hydrogen atom or group of atoms (i.e., a “substituent”), and/or the alkyl is interrupted by one or more substituents comprising heteroatoms selected from O, S and NR′, wherein R′ is as defined below. Non-limiting examples of groups that may replace a hydrogen atom include halogen; alkyl groups; cycloalkyl groups; oxo groups (═O); hydroxyl groups (—OH); —(C═O)OR′; —O(C═O)R′; —C(═O)R′; —OR′; —S(O)ₓR′; —S—SR′; —C(═O)SR′; —SC(═O)R′; —NR′R′; —NR′C(═O)R′; —C(═O)NR′R′; —NR′C(═O)NR′R′; —OC(═O)NR′R′; —NR′C(═O)OR′; —NR′S(O)ₓNR′R′; —NR′S(O)ₓR′; and —S(O)ₓNR′R′, wherein R′ at each occurrence is independently selected from H, C₁-C₁₅ alkyl or cycloalkyl, and x is 0, 1 or 2.

As used herein, the term “lipophilic chain” refers to an alkyl group bonded to a nitrogen or carbon atom of the lipid, said alkyl group comprising at least 6 C atoms and optionally comprising C═C double bonds, and/or ring structures, and/or carbonyl groups, and/or heteroatoms such as N, O, S, and such that the parent compound of said alkyl group has a C Log P of at least 6.

For example, lipid MC3, 1, and lipid KC2, 2, have a pair of lipophilic chains derived from (6Z,9Z)-octadeca-6,9-diene, which has a C Log P of 9.25:

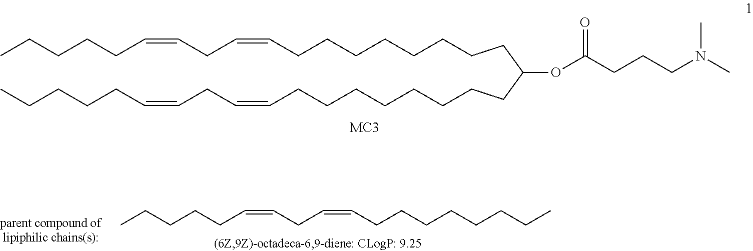

Lipid ALC-0315, 3, has a pair of lipophilic chains derived from hexyl 2-hexyldecanoate, which has a C Log P of 10.01:

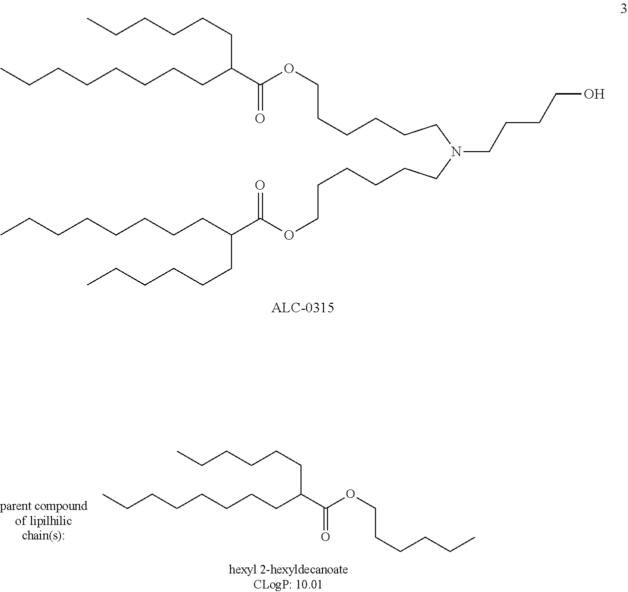 [SMILES: CCCCCCCCC(CCCCCC)C(=O)OCCCCCCN(CCCCO)CCCCCCOC(=O)C(CCCCCC)CCCCCCCC ; CCCCCCCCC(CCCCCC)C(=O)OCCCCCC]

Lipid SM-102, 4, has one lipophilic chain derived from undecyl hexanoate, which has a C Log P of 7.59, and one lipophilic chain derived from heptadecane-9-yl octanoate, which has a C Log P of 11.6:

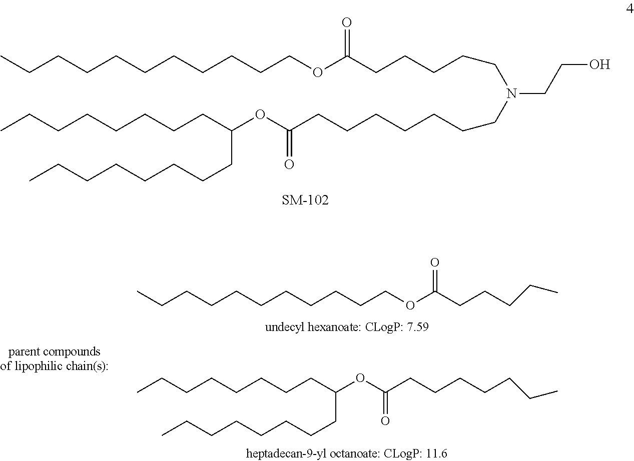

As used herein, the term “helper lipid” means a compound selected from: a sterol such as cholesterol or a derivative thereof, a diacylglycerol or a derivative thereof, such as a glycerophospholipid, including phosphatidic acid (phosphatidate) (PA), phosphatidylethanolamine (cephalin) (PE), phosphatidylcholine (PC), phosphatidylserine (PS), and the like; and a sphingolipid, such as a ceramide, a sphingomyelin, a cerebroside, a ganglioside, or reduced analogues thereof, that lack a double bond in the sphingosine unit. An example of a diacylglycerol derivative is a glycerophospholipid-cholesterol conjugate in which one of the acyl chains is substituted with a moiety comprising cholesterol. The term encompasses lipids that are either naturally-occurring or synthetic.

As used herein, the term “delivery vehicle” includes any preparation in which the lipid described herein is capable of being formulated and includes but is not limited to delivery vehicles comprising helper lipids.

As used herein, the term “nanoparticle” is any suitable particle in which the lipid can be formulated and that may comprise one or more helper lipid components. The one or more lipid components may include an ionizable lipid prepared by the method described herein and/or may include additional lipid components, such as the one or more helper lipid components. The term includes, but is not limited to, vesicles with one or more bilayers, including multilamellar vesicles, unilamellar vesicles and vesicles with an electron-dense core. The term also includes polymer-lipid hybrids, including particles in which the lipid is attached to a polymer.

As used herein, the term “encapsulated,” with reference to incorporating a cargo molecule (e.g., mRNA) within a delivery vehicle refers to any association of the cargo with any component or compartment of the delivery vehicle such as a nanoparticle.

The term “pharmaceutically acceptable salt” with reference to a form of the lipid of the disclosure in a protonated form (i.e., charged) and/or as part of a pharmaceutical formulation in which an LNP is formulated refers to a salt of the lipid prepared from pharmaceutically acceptable acids, including inorganic and organic acids.

The article “a” or “an” as used herein is meant to include both singular and plural, unless otherwise indicated.

### SUMMARY

The present disclosure is based, at least in part, on the surprising discovery that LNP formulations of nucleic acids comprising ionizable lipids that incorporate certain sulfur-containing moieties, for example, dithioacetal and dithioketal moieties, in one or more of their lipophilic chains are more potent than the benchmark MC3 for liver or spleen delivery of nucleic acid, such as RNA. In some embodiments, the lipids of the disclosure exhibit a different organ selectivity relative to known lipids. In particular, certain embodiments described herein promote delivery of nucleic acid, such as mRNA, selectively to the spleen more efficiently than known lipids. In addition, the chemical synthesis of the lipids of certain embodiments herein is more straightforward and/or economical than that of known lipids.

According to one aspect of the disclosure, there is provided a lipid having a structure of Formula A:

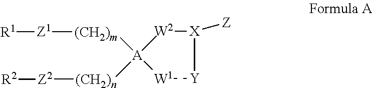

or a pharmaceutically acceptable salt thereof,
wherein
R¹ and R² are, independently, a linear or branched alkyl group comprising between six and twenty C atoms, optionally substituted, optionally comprising C═C double bonds of E or Z geometry, and optionally comprising heteroatoms selected from N, O or S, wherein
at least one of R¹ or R² comprises at least one S atom, wherein
if two or more S atoms are present in R¹ and/or R², the two or more S atoms are either bound to the same or to different respective carbon atoms, and wherein
the two or more S atoms are not bound to each other,
Z¹ and Z² are each a carboxy group

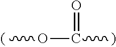

and wherein the C atom of the carboxy group is bound to the (CH₂)ₘ or (CH₂)ₙ moiety, and the O atom of the carboxy group is bound to the R¹ or R² group, or wherein the O atom of the carboxy group is bound to the (CH₂)ₘ or (CH₂)ₙ moiety, and the C atom of the carboxy group is bound to the R¹ or R² group;
m and n are, independently, 4 to 8;
A is either C or N, and if A is C, then
W¹ and Y are either bonded to each other or not bonded to each other, as indicated by the dashed bond, wherein
   if W and Y are bonded to each other, then
      W¹ is O or S
      W² is O or S
      X is CH
      Y is (CH₂)ₘ, wherein m is 1 or 2; and
      Z is a group selected from structures a-c below, wherein the wavy line represents
      the bond to X:
      a.

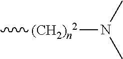

type 2 ionizable head;
b.

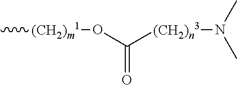

type 3 ionizable head; and
c.

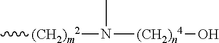

type 4 ionizable head,
if W and Y are not bonded to each other, then
   W¹ is H;
   W² is O or S or NH or NR², wherein R² is a C₁ to C₄ alkyl optionally substituted with an OH group; and
   Group

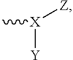

wherein the wavy line represents the bond to W², is a group selected from structures d-h below, wherein the wavy line represents the bond to W².
d.

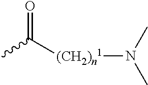

if W² is O, type 1 ionizable head;
e.

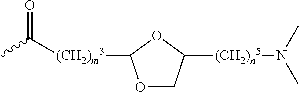

if W² is O, type 5 ionizable head;
f.

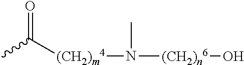

if W² is O, type 6 ionizable head;
g. [symbol] (CH₂)ₙ_{⁷}—OH if W² is NH or NR², type 7 ionizable head;
h.

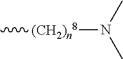

if W² is NH or NR², type 8 ionizable head; and
i.

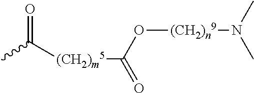

if W² is O, type 9 ionizable head,
if A is N, then
   W¹ and Y are absent
   W² and X together form a group of structure (CR^{a}R^{b})ₚ, wherein R^{a} and R^{b} are independently selected from H, C₁-C₅ alkyl or C₁-C₅ cycloalkyl, and wherein index p is 2 to 6; and
   Z is OH or NR′R″, wherein R′ and R″ are independently C₁-C₅ alkyl, C₁-C₅ cycloalkyl, or moieties of a heterocyclic group that incorporates the N atom to which R′ and R″ are bound.

According to one embodiment of the foregoing aspect, the A is N, W¹ and Y are absent, W² and X together form a group of the structure (CR^{a}R^{b})ᵣ, and Z is NR′R″ and wherein R′ and R″ are moieties of the heterocyclic group that incorporates the N atom to which R′ and R″ are bound and wherein the heterocyclic group is selected from pyrrolidine, piperidine or morpholine.

According to the above aspect or embodiments thereof, the A may be a carbon atom.

According to the above aspect or embodiments thereof, the W¹ and Y may not be bonded to each other.

According to the above aspect or embodiments thereof, the W² may be O.

According to the above aspect or embodiments thereof, the moiety

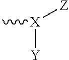

of Formula A is structure d.

According to the above aspect or embodiments thereof, the W² may be NR³ and wherein R³ is substituted with an OH group.

According to the above aspect or embodiments thereof, Z may be structure g.

According to the above aspect or embodiments thereof, W¹ and Y may be bonded to each other.

According to the above aspect or embodiments thereof, Z may be structure a.

In one embodiment, the lipid has a structure of any one of the compounds 5-19 or pharmaceutically acceptable salts thereof as set forth in Table 1 hereinafter.

According to another aspect of the disclosure, there is provided a lipid or a pharmaceutically acceptable salt thereof comprising:
a protonatable amino head group;
at least two lipophilic chains, wherein the protonatable amino head group has a central nitrogen atom or carbon atom to which each of the two lipophilic chains are directly bonded;
at least one of the lipophilic chains has the formula:

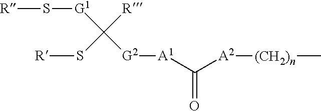

wherein R′ and R″ are, independently, linear or branched optionally substituted C₃-C₁₂ alkyl and optionally with varying degrees of unsaturation;
R′″ is H or a linear, branched, or cyclic optionally substituted C₁-C₆ alkyl group;
G¹ and G² are, independently, a group of (CR^{a}R^{b})ₚ, wherein R^{a} and R^{b} are independently selected from H or optionally substituted C₁-C₅ alkyl or cycloalkyl, and wherein p is 0 to 6;
one of A¹ and A² is an O, and a respective other one of A¹ and A² is a bond, or wherein A¹ and A² are both O;
n is 4 to 8;
each lipophilic chain has between 15 and 30 carbon atoms in total; and
wherein the lipid has (i) a pKₐ of between 6 and 8; and (ii) a log P of at least 11.

According to another embodiment of any of the above aspects or embodiments, the lipid, when formulated in a lipid nanoparticle comprising an mRNA encoding for luciferase, results in an increase in luminescence intensity per mg of organ in vivo of at least about 10% as measured in the liver and/or spleen relative to an otherwise identical lipid nanoparticle containing a DLin-MC3-DMA ionizable lipid. The formulations are prepared and the biodistribution assay is carried out as described in Example 2 hereinafter.

According to another aspect of the disclosure, there is provided a lipid nanoparticle comprising the lipid as described in any aspect or embodiment above and a nucleic acid.

In another embodiment, the lipid nanoparticle comprises a helper lipid and optionally a hydrophilic polymer-lipid conjugate. The helper lipid may be selected from cholesterol, a diacylglycerol, a glycerophospholipid-cholesterol conjugate and a sphingolipid.

According to another aspect of the disclosure, there is provided a lipid nanoparticle comprising:
an ionizable lipid with at least two lipophilic chains, at least one of the two lipophilic chains having a dithioacetal and dithioketal moiety;
one or more helper lipids;
optionally a hydrophilic polymer-lipid conjugate; and
a nucleic acid.

According to a further aspect of the disclosure, there is provided a method for administering a nucleic acid to a subject in need thereof, the method comprising preparing or providing the lipid nanoparticle of any one of the foregoing aspects or embodiments comprising the nucleic acid and administering the lipid nanoparticle to the subject.

According to a further aspect of the disclosure, there is provided a method for delivering a cargo molecule to a cell, the method comprising contacting the lipid nanoparticle of any one of the foregoing aspects or embodiments with the cell in vivo or in vitro. The cargo molecule may be a nucleic acid.

According to a further aspect of the disclosure, there is provided use of the lipid or the pharmaceutically acceptable salt thereof of any one of the aspects or embodiments above or the lipid nanoparticle of any one of the aspects or embodiments above in the manufacture of a medicament to treat or prevent a disease, disorder or condition that is treatable and/or preventable by a nucleic acid.

According to a further aspect of the disclosure, a use of the lipid or the pharmaceutically acceptable salt thereof of any one of the aspects or embodiments above or the lipid nanoparticle of any one of the aspects or embodiments above to deliver a nucleic acid to a patient to treat or prevent a disease, disorder or condition that is treatable or preventable by the nucleic acid.

Other objects, features, and advantages of the present disclosure will be apparent to those of skill in the art from the following detailed description and figures.

### BRIEF DESCRIPTION OF THE DRAWINGS

FIG. 1 is a bar graph showing entrapment (%), particle size and polydispersity index (PDI) of mRNA-containing lipid nanoparticles (LNPs) comprising the ionizable lipids 1, 5-15, 18 and 19. The LNPs are composed of 50/10/38.5/1.5 mol % of ionizable lipid/DSPC/chol/PEGDMG and the amine-to-phosphate ratio (N/P) was 6.

FIG. 2A shows luminescence intensity/mg in the liver for the mRNA-containing LNPs comprising the ionizable lipids 1, 5-15, 18 and 19 after 4 hours post-intravenous administration to CD-1 mice. The LNPs contain 50/10/38.5/1.5 mol % of ionizable lipid/DSPC/chol/PEG-DMG (N/P=6).

FIG. 2B shows luminescence intensity/mg in the spleen for the mRNA-containing LNPs comprising the ionizable lipids 1, 5-15, 18 and 19 after 4 hours post-intravenous administration to CD-1 mice. The LNPs contain 50/10/38.5/1.5 mol % of ionizable lipid/DSPC/chol/PEG-DMG (N/P=6).

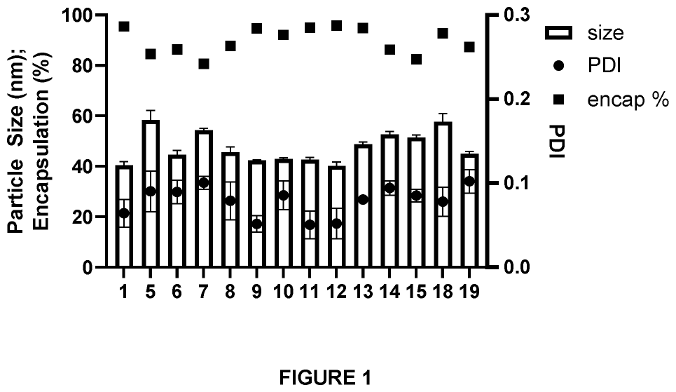

### DETAILED DESCRIPTION

Various aspects and embodiments of the disclosure are directed to ionizable lipids having structures of Formula A and pharmaceutically acceptable salts thereof.

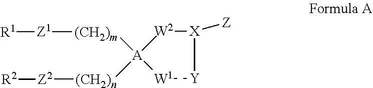

Formulations comprising such lipids find use in the delivery of nucleic acid to any target site of interest. In some embodiments, such lipids have been found to be particularly efficacious for the delivery of mRNA when formulated in a suitable delivery vehicle. In further embodiments, such lipids can be easily synthesized and prepared by processes having improved economics relative to known methods for making ionizable lipids.

### Methods to Produce Lipids of Formula A

Lipids of Formula A or pharmaceutically acceptable salts thereof can be prepared using any suitable method known to those of skill in the art. Particularly suitable methods are described below and are exemplified, without intending to be limiting, with the synthesis of compound 5-19 of Table 1.

TABLE 1

| | |
|---|---|
| 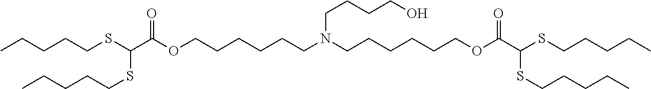 [SMILES: CCCCCSC(SCCCCC)C(=O)OCCCCCCN(CCCCO)CCCCCCOC(=O)C(SCCCCC)SCCCCC] | 5 |
| 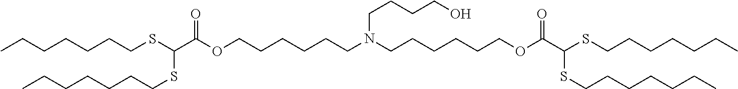 [SMILES: CCCCCCCSC(SCCCCCCC)C(=O)OCCCCCCN(CCCCO)CCCCCCOC(=O)C(SCCCCCCC)SCCCCCCC] | 6 |
| 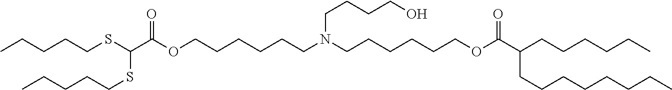 [SMILES: CCCCCCCCC(CCCCCC)C(=O)OCCCCCCN(CCCCO)CCCCCCOC(=O)C(SCCCCC)SCCCCC] | 7 |
| 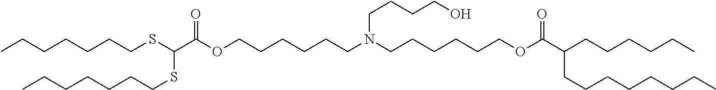 [SMILES: CCCCCCCCC(CCCCCC)C(=O)OCCCCCCN(CCCCO)CCCCCCOC(=O)C(SCCCCCCC)SCCCCCCC] | 8 |
| 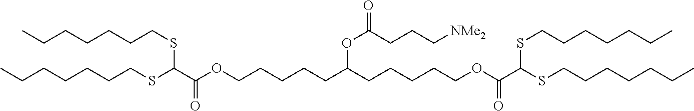 [SMILES: CCCCCCCSC(SCCCCCCC)C(=O)OCCCCCC(CCCCCOC(=O)C(SCCCCCCC)SCCCCCCC)OC(=O)CCCN(C)C] | 9 |
| 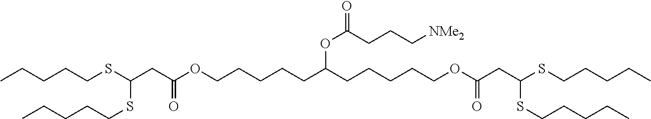 [SMILES: CCCCCSC(CC(=O)OCCCCCC(CCCCCOC(=O)CC(SCCCCC)SCCCCC)OC(=O)CCCN(C)C)SCCCCC] | 10 |
| 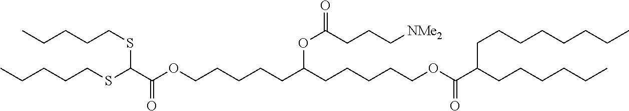 [SMILES: CCCCCCCCC(CCCCCC)C(=O)OCCCCCC(CCCCCOC(=O)C(SCCCCC)SCCCCC)OC(=O)CCCN(C)C] | 11 |
| 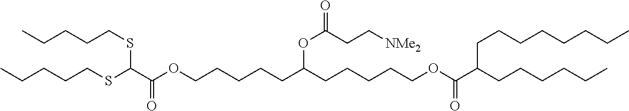 [SMILES: CCCCCCCCC(CCCCCC)C(=O)OCCCCCC(CCCCCOC(=O)C(SCCCCC)SCCCCC)OC(=O)CCN(C)C] | 12 |
| 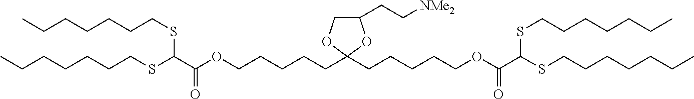 [SMILES: CCCCCCCSC(SCCCCCCC)C(=O)OCCCCCC1(CCCCCOC(=O)C(SCCCCCCC)SCCCCCCC)OCC(CCN(C)C)O1] | 13 |
| 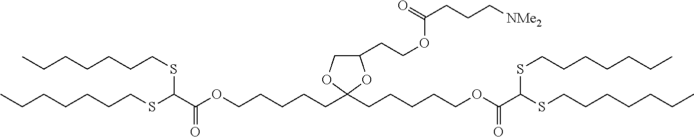 [SMILES: CCCCCCCSC(SCCCCCCC)C(=O)OCCCCCC1(CCCCCOC(=O)C(SCCCCCCC)SCCCCCCC)OCC(CCOC(=O)CCCN(C)C)O1] | 14 |
| 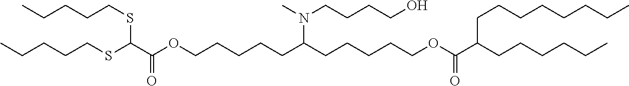 [SMILES: CCCCCCCCC(CCCCCC)C(=O)OCCCCCC(CCCCCOC(=O)C(SCCCCC)SCCCCC)N(C)CCCCO] | 15 |
| 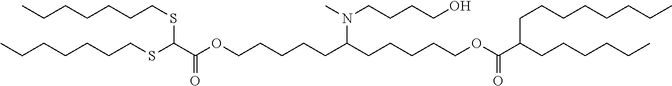 [SMILES: CCCCCCCCC(CCCCCC)C(=O)OCCCCCC(CCCCCOC(=O)C(SCCCCCCC)SCCCCCCC)N(C)CCCCO] | 16 |
| 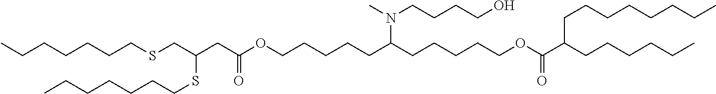 [SMILES: CCCCCCCCC(CCCCCC)C(=O)OCCCCCC(CCCCCOC(=O)CC(CSCCCCCCC)SCCCCCCC)N(C)CCCCO] | 17 |
| 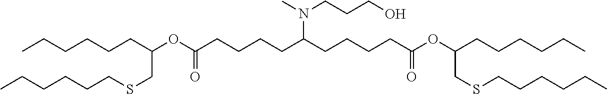 [SMILES: CCCCCCSCC(CCCCCC)OC(=O)CCCCC(CCCCC(=O)OC(CCCCCC)CSCCCCCC)N(C)CCCO] | 18 |
| 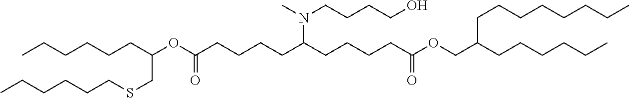 [SMILES: CCCCCCCCC(CCCCCC)COC(=O)CCCCC(CCCCC(=O)OC(CCCCCC)CSCCCCCC)N(C)CCCCO] | 19 |

Those skilled in the art would appreciate that alternative starting materials could be employed in the same sequence, leading to congeners of compound 5-19 as defined by Formula A. Therefore, the synthetic schemes set forth below are merely illustrative of select embodiments.

The synthesis of representative, but nonlimiting, lipids 5-19 requires sulfur-containing fragments 20-24 (Scheme 2). Such fragments can be prepared using any suitable method known to those of skill in the art. Particularly suitable methods are described below. Those skilled in the art would appreciate that alternative starting materials could be employed in the same sequences, leading to congeners of compound 20-24, and consequently to diverse analogues of lipids 5-19. Therefore, the schemes set forth below are merely illustrative of select embodiments.

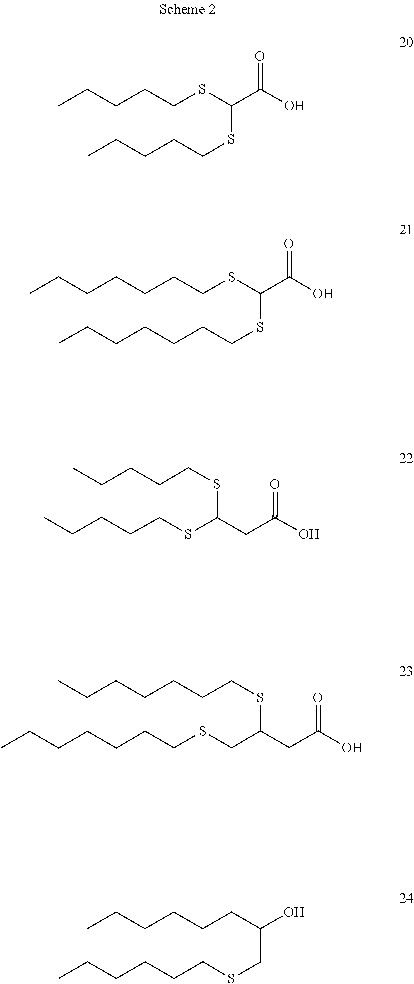 [SMILES: CCCCCSC(SCCCCC)C(=O)O ; CCCCCCCSC(SCCCCCCC)C(=O)O ; CCCCCSC(CC(=O)O)SCCCCC ; CCCCCCCSCC(CC(=O)O)SCCCCCCC ; CCCCCCSCC(O)CCCCCC]

Compounds 20 and 21 can be prepared by the method of Bates, et al. (Can J. Chem. 1980, 58, 716; incorporated herein by reference). Thus, reaction of glyoxylic acid monohydrate with a thiol in the presence of a catalytic amount of a protonic acid such as para-toluenesulfonic acid, produces the desired substances (Scheme 3).

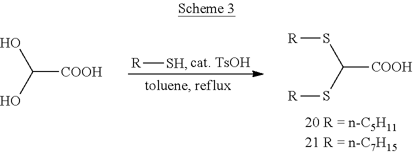

Compounds such as 22 can be prepared by the reaction of an aldehydoacid, aldehydoester, aldehydonitrile, or an acetal derivative of such compounds, with an appropriate thiol, followed by ester or nitrile hydrolysis. For example, 22 itself can be made from 25 as shown in Scheme 4.

Compounds such as 23 can be prepared by the reaction of a 4-bromocrotonate ester with an

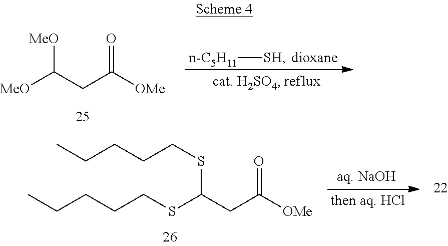

appropriate thiol, followed by ester saponification. Thus, 23 itself can be made from methyl 4-bromocrotonate, 27, as shown in Scheme 5.

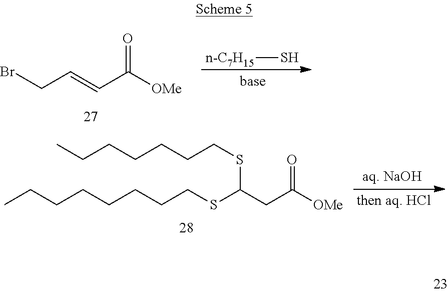

Compounds such as 24 can be prepared by reaction of an epoxide with a thiol under basic conditions. Thus, 24 itself can be made from 1-octene oxide, 29, as shown in Scheme 6.

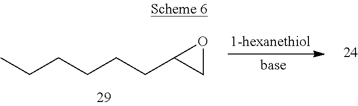

Certain steps of the synthesis of lipids such as 5-8 are described in detail in co-pending and co-owned WO 2023/173203, which is incorporated herein by reference. As described in the foregoing disclosure, one such step entails reacting an aminoalcohol, or an O-protected variant thereof, with a suitable alkyl halide or sulfonate, in an appropriate solvent, resulting in formation of different products depending on the conditions.

Specifically, a primary amine represented in Scheme 7 with the generic formula 30 can be doubly N-alkylated in a single step by reaction with about two molar equivalents of an alkyl halide or sulfonate represented in Scheme 2 with the generic formula 31, in acetonitrile, at a suitably elevated temperature, and in the presence of a base such as K₂CO₃ or Na₂CO₃, whereupon the starting primary amine 30 is converted into a tertiary amine of generic structure 32. In cases where Z in 32 is a protecting group, a deprotection step can be used to free the OH group.

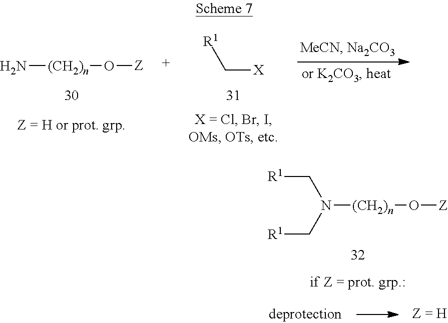

Alternatively (Scheme 8), primary amine 30 can be converted into a corresponding secondary amine of generic formula 33 by reaction with about one equivalent of the alkyl halide or sulfonate of generic formula 31, in DMF, at or below room temperature, and in the presence of K₂CO₃. Secondary amine 33 can subsequently react with a suitable alkyl halide or sulfonate of generic formula 34, in acetonitrile, at a suitably elevated temperature, and in the presence of a base such as K₂CO₃ or Na₂CO₃, whereupon 33 is converted into a tertiary amine of generic structure 35. In cases where Z in 35 is a protecting group, a deprotection step can be used to free the OH group.

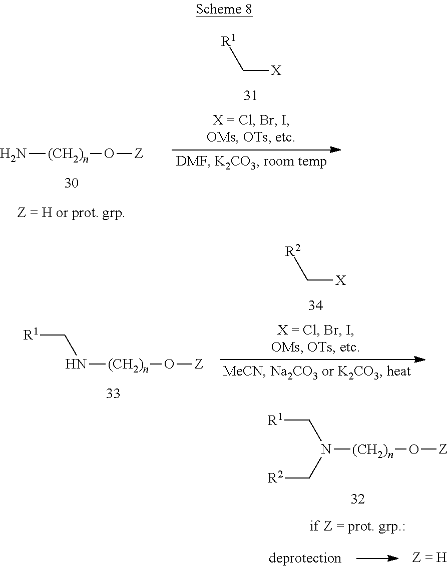

In accordance with the foregoing, representative, but nonlimiting, lipids 5-8 can be made using, for example, alkyl bromides 36-38 (Scheme 9) in the alkylation reactions of Schemes 7 and 8.

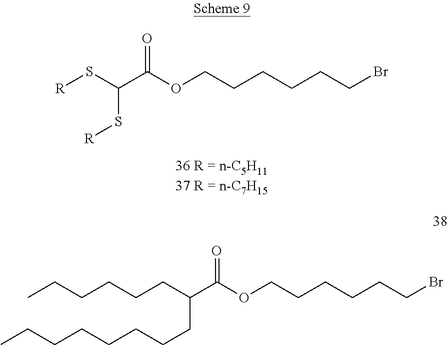

Compounds 36-38 and congeners can be prepared, for example, by esterification of 20, 21, and 2-hexyldecanoic acid, respectively, with a haloalcohol such as 6-bromo-1-hexanol in the presence of a condensing agent such as a carbodiimide, for example, EDCI, and optionally in the presence of DMAP. As described in co-owned and co-pending WO 2023/173203, an alternative way to produce esters such as 36 entails the Fischer esterification of an acid such as 2-hexyldecanoic acid with a haloalcohol such as 6-bromo-1-hexanol in the presence of an acid catalyst; for example, sulfuric acid, para-toluenesulfonic acid, and the like. Furthermore, the cited Application teaches that sulfonate (OMs, OTs, etc.) analogues of 36-38 can be prepared, for example, by mono-esterification of the corresponding acids with a diol such as 1,6-hexanediol, either in the presence of a condensing agent such as a carbodiimide, for example, EDCI, and optionally in the presence of DMAP, or by the Fischer method described above, followed by conversion of the hydroxyester products into appropriate sulfonate esters.

The method of Scheme 7 can be used to prepare lipids 5 and 6. The synthesis of lipid 5 can thus be achieved by the double N-alkylation of 4-amino-1-butanol by reaction, for example, with alkyl bromide 36 in acetonitrile, at 80° C. and in the presence of Na₂CO₃(Scheme 10).

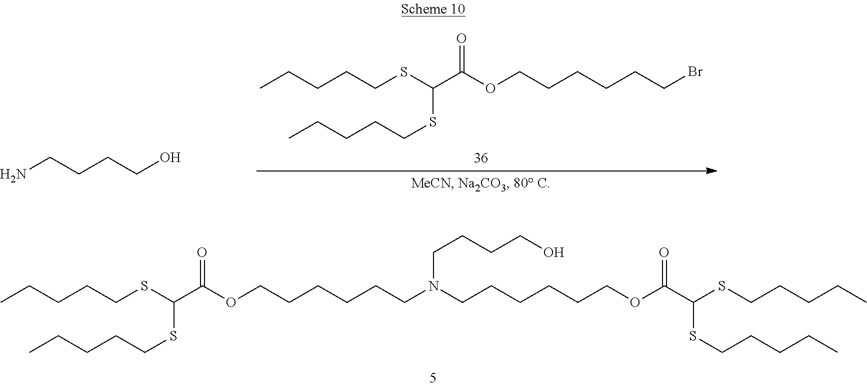 [SMILES: CCCCCSC(SCCCCC)C(=O)OCCCCCCBr ; NCCCCO ; CCCCCSC(SCCCCC)C(=O)OCCCCCCN(CCCCO)CCCCCCOC(=O)C(SCCCCC)SCCCCC]

The synthesis of lipid 6 can be achieved by double N-alkylation of 4-amino-1-butanol with, for example, alkyl bromide 37 (Scheme 11).

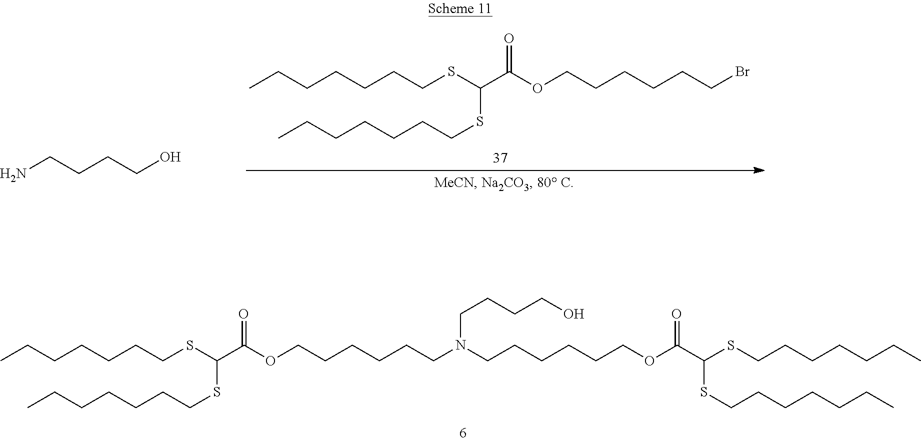 [SMILES: CCCCCCCSC(SCCCCCCC)C(=O)OCCCCCCBr ; NCCCCO ; CCCCCCCSC(SCCCCCCC)C(=O)OCCCCCCN(CCCCO)CCCCCCOC(=O)C(SCCCCCCC)SCCCCCCC]

The method of Scheme 8 can be used to prepare lipids 7 and 8. The synthesis of lipid 7 can thus be achieved by the mono N-alkylation of 4-amino-1-butanol by reaction, for example, with alkyl bromide 36 in DMF, at or below room temperature in the presence of K₂CO₃, leading to formation of secondary amine 39. Subsequent reaction of 39 with, for example, alkyl bromide 38 in MeCN at 80° C. in the presence of Na₂CO₃ produces 7 (Scheme 12).

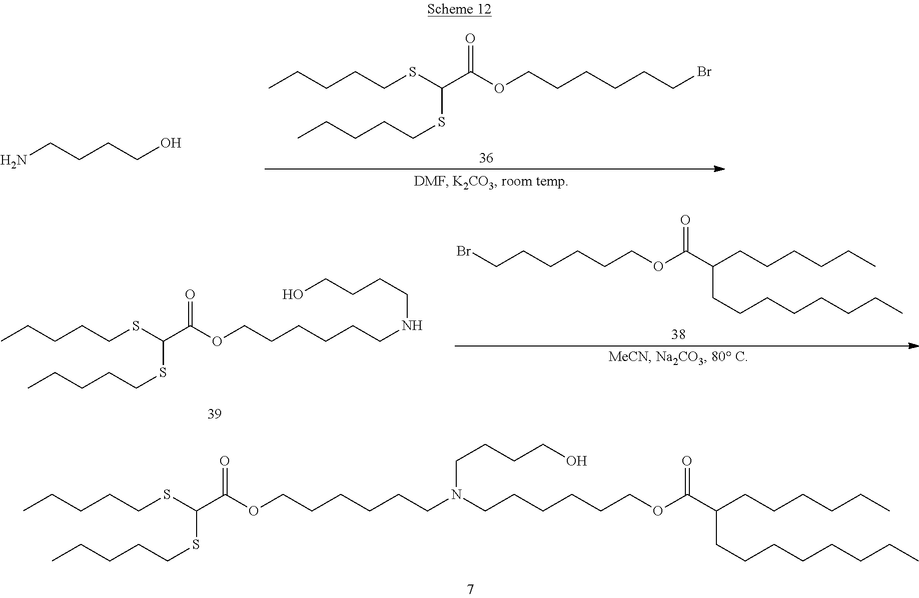 [SMILES: CCCCCSC(SCCCCC)C(=O)OCCCCCCBr ; NCCCCO ; CCCCCSC(SCCCCC)C(=O)OCCCCCCNCCCCO ; CCCCCCCCC(CCCCCC)C(=O)OCCCCCCBr ; CCCCCCCCC(CCCCCC)C(=O)OCCCCCCN(CCCCO)CCCCCCOC(=O)C(SCCCCC)SCCCCC]

Furthermore, the order of the steps shown in Scheme 12 can be reversed, meaning that 4-amino-1-butanol can be made to react first with, for example, alkyl bromide 38 in DMF at room temperature and in the presence of K₂CO₃, followed by reaction of the resulting secondary amine 40 with, foe example, alkyl bromide 36 in MeCN at 80° C. in the presence of Na₂CO₃ (Scheme 13).

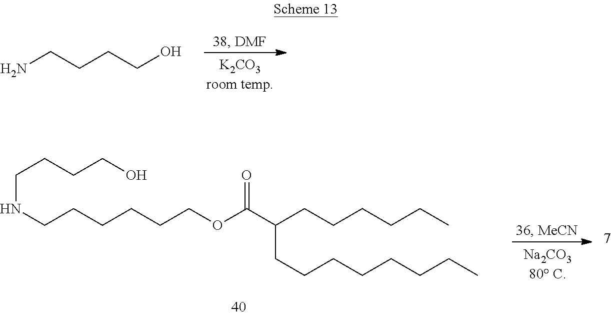 [SMILES: NCCCCO ; CCCCCCCCC(CCCCCC)C(=O)OCCCCCCNCCCCO]

Likewise, lipid 8 can be made by mono N-alkylation of 4-amino-1-butanol with, for example, alkyl bromide 37 in DMF, at room temperature and in the presence of K₂CO₃, followed by N-alkylation of the resulting 41 with, for example, alkyl halide 38 in MeCN, at 80° C. and in the presence of Na₂CO₃ (Scheme 14), or by mono N-alkylation of 4-amino-1-butanol with, for example, 38 in

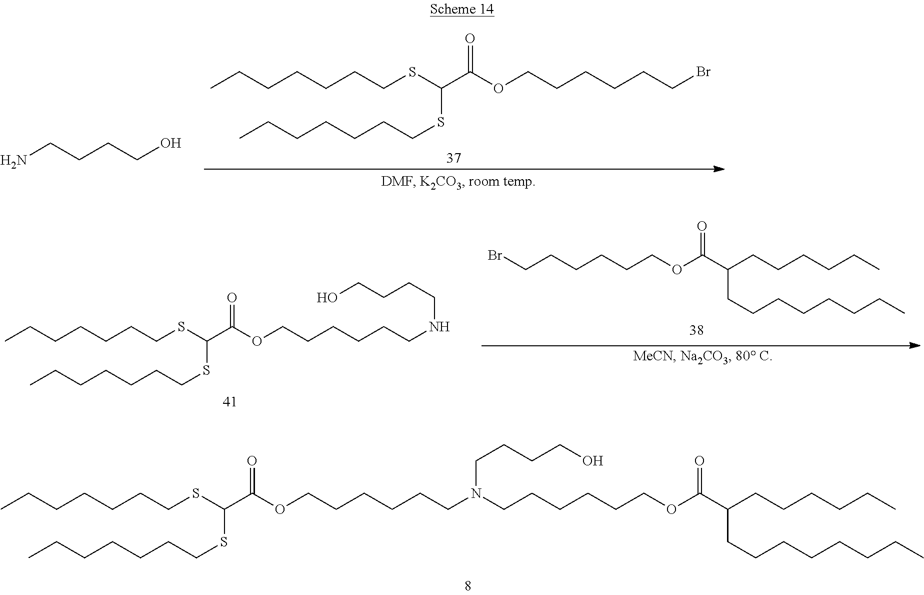 [SMILES: CCCCCCCSC(SCCCCCCC)C(=O)OCCCCCCBr ; NCCCCO ; CCCCCCCSC(SCCCCCCC)C(=O)OCCCCCCNCCCCO ; CCCCCCCCC(CCCCCC)C(=O)OCCCCCCBr ; CCCCCCCCC(CCCCCC)C(=O)OCCCCCCN(CCCCO)CCCCCCOC(=O)C(SCCCCCCC)SCCCCCCC]

DMF, at room temperature, in the presence of K₂CO₃, followed by N-alkylation of the resulting 42 with, for example, 37 in MeCN, at 80° C. and the presence of Na₂CO₃ (Scheme 16).

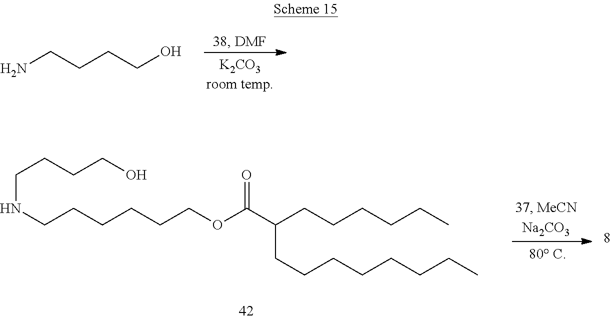 [SMILES: NCCCCO ; CCCCCCCCC(CCCCCC)C(=O)OCCCCCCNCCCCO]

The person skilled in the art will recognize that diverse congeners of lipids 5-8 can be prepared by the methods outlined above using alternative starting materials.

The skilled artisan will also appreciate that the ionizable head group present in lipids 9-19 of Table 1 can be introduced starting with a precursor of said lipids, wherein a ketone functionality is present in lieu of the ionizable head group. The ketone can then be transformed into a suitable ionizable head group by appropriate organic synthesis steps. Therefore, the synthesis of 9-19 starts with the preparation of an appropriate ketone.

Representative, but non-limiting, lipids of Table 1 can be made from dihydroxyketones of general structure 43 and/or from ketodiacids of general structure 44 (Scheme 16).

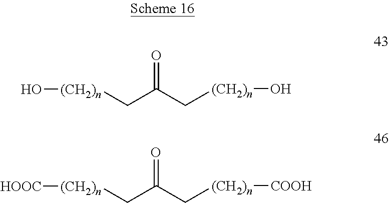

Certain steps of the synthesis of ketones such as 43 and 44 are described in detail in co-pending and co-owned WO 2023/147657, incorporated herein by reference. As described in said disclosure, a ketone such as 43 can be made by subjecting an appropriate lactone to Claisen condensation under Mukaiyama conditions, followed by hydrolysis of the resulting beta-ketolactone and decarboxylation of the intermediate beta-ketoacid, leading to the formation of a dihydroxyketone ketone.

In certain embodiments, these steps are most advantageously carried out in a “one-pot operation”, meaning that the various synthetic intermediates, while isolable, need not be isolated.

For example, the synthesis of certain lipids of Table 1 requires dihydroxyketone 47, a compound of the type 43 wherein n=4. This substance can be prepared from caprolactone as shown in Scheme 17, in which case the synthetic intermediates that optionally need not be isolated are compounds 45 and 46.

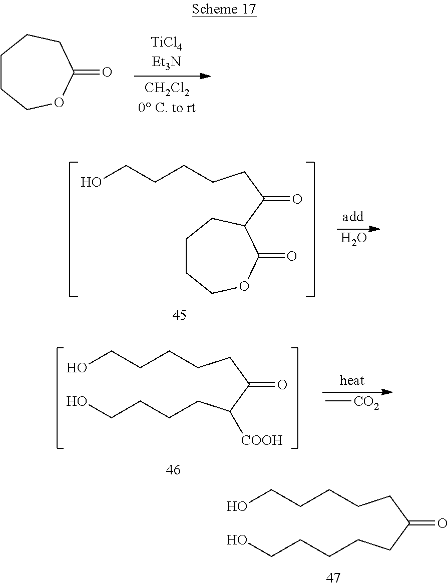

Alternatively, the foregoing PCT Application teaches that a ketone of general structure 43 can be synthesized by subjecting an appropriately O-protected derivative of a hydroxyester to Claisen condensation under Mukaiyama conditions, followed by hydrolysis of the resulting beta-ketoester, release of the O-protecting groups, and decarboxylation, resulting in formation of a ketodiol.

In certain embodiments, these steps of hydrolysis of the beta-ketoester, release of the O-protecting groups, and decarboxylation, are most advantageously carried out in a “one-pot operation”, meaning that the various synthetic intermediates, while isolable, need not be isolated.

For example, dihydroxyketone 51, a compound of the type 43 wherein n=6, can be made from O-protected hydroxyester 48, and the synthetic intermediates that optionally needs not be isolated are compounds 49 and 50 (Scheme 18).

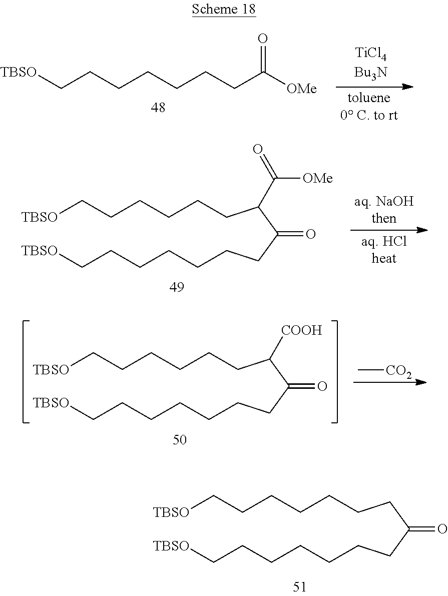

As described in the foregoing PCT Application, a ketone of general structure 44 can be made by converting the monoester of a dicarboxylic acid to the corresponding acid chloride, followed by treatment of the acid chloride with an appropriate weak base (see Durham, L. J., et al., Org. Synth. 1958, 38, 38; incorporated herein by reference). This results in formation of a beta-lactone product, described as a ketene dimer, which can be subjected to hydrolysis of the lactone and decarboxylation of the resultant beta-ketoacid to produce a ketodiester.

In certain embodiments, the steps of hydrolysis of the beta-lactone and decarboxylation are most advantageously carried out under conditions that also induce hydrolysis of the ester groups.

In certain embodiments, the steps of hydrolysis of the beta-lactone and decarboxylation and hydrolysis of the ester groups are most advantageously carried out in a “one-pot operation”, meaning that the various synthetic intermediates, while isolable, need not be isolated.

For example, ketodiacid 55, a compound of the type 44 wherein n=3, can be obtained from monoethyl adipate by way of acid chloride 52, ketene dimer 53, wherein the wavy bond signifies that the C═C double bond may be of E or Z geometry, and triacid 54, and the synthetic intermediates that optionally needs not be isolated are 53 and 54 (Scheme 19).

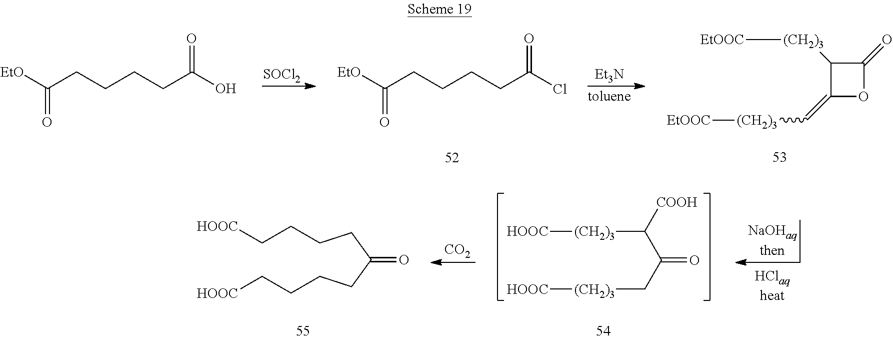

Ketones of general structure 43 or 44, as exemplified by 47 (Scheme 17) and 55 (Scheme 19), are symmetrical, meaning that the alkyl groups bonded to the carbonyl are identical. As described in co-owned and co-pending U.S. provisional patent application No. 63/445,854, unsymmetrical congeners of 43 and 44, that is, ketones such as 58, wherein two different alkyl groups are bonded to the carbonyl group, can be prepared by sequential alkylation of TosMIC with two different alkyl halides or sulfonates, followed by hydrolysis of the product under acidic conditions (Scheme 20). The individual skilled in the art will appreciate that diverse unsymmetrical analogues of the lipids of Table 1 are thus accessible from unsymmetrical ketones of the type 58 by obvious modifications of the synthetic schemes provided below.

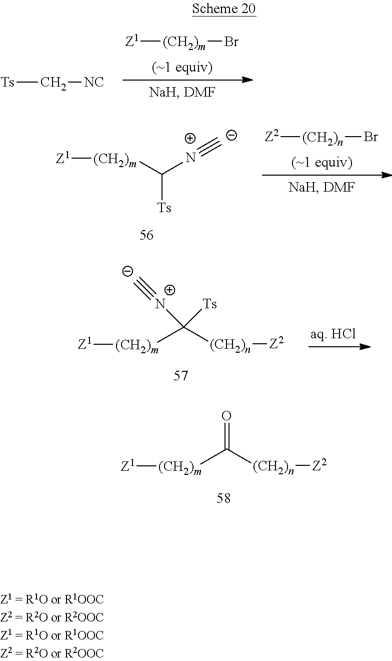

The conversion of a dihydroxyketone of general structure 43 into a lipid of Formula A starts with the esterification of the OH groups with appropriate carboxylic acids. As described in the foregoing WO 2023/147657, the esterification reaction can be carried out so that a symmetrical diester of general structure 59 is formed as the major product, for example, by the use of at least 2 molar equivalents of an acid R′—COOH in the presence of a condensing agent, for example, a carbodiimide such as EDCI, and DMAP, or so that monoester 60 is formed as the major product, for example, by the use of about 1 molar equivalent of an acid R′—COOH in the presence of a condensing agent, for example, a carbodiimide such as EDCI, and DMAP. Furthermore, monoester 60 can be converted into unsymmetrical diester 61 by subsequent reaction with a second carboxylic acid, R″—COOH, in the presence of a condensing agent, for example, a carbodiimide such as EDCI, and DMAP (Scheme 21). The ketone group in 59 or 61 can then be transformed into an ionizable head group of type 1-9 (see definitions above) by chemical methods that are well known to those skilled in the art, such as the exemplary methods provided herein.

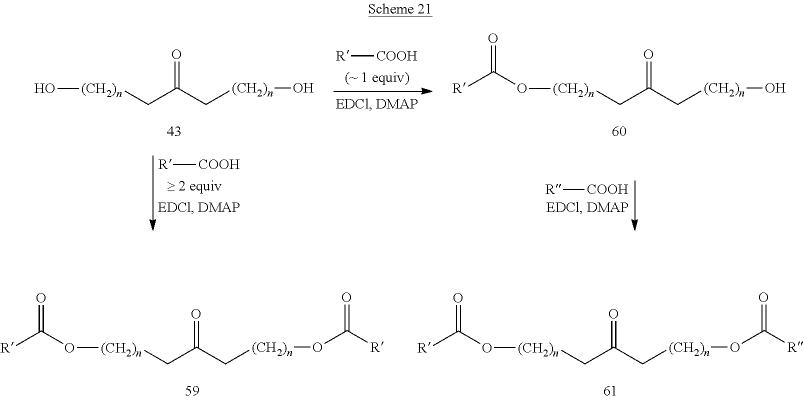

In a like manner, the conversion of a ketodiacid of general structure 44 into a lipid of Formula A starts with the esterification of the COOH groups with appropriate alcohols. As described in the foregoing WO 2023/147657, the esterification reaction can be carried out so that a symmetrical diester of general structure 62 is formed as the major product, for example, by the use of at least 2 molar equivalents of an alcohol R′—OH in the presence of a condensing agent, for example, a carbodiimide such as EDCI, and DMAP, or so that monoester 63 is formed as the major product, for example, by the use of about 1 molar equivalent of an alcohol R′—OH in the presence of a condensing agent, for example, a carbodiimide such as EDCI, and DMAP. Furthermore, monoester 63 can be converted into unsymmetrical diester 64 by subsequent reaction with a second alcohol, R″—OH, in the presence of a condensing agent, for example, a carbodiimide such as EDCI, and DMAP (Scheme 22). The ketone group in 62 or 64 can then be transformed into an ionizable head group of type 1-9 (see definitions above) by chemical methods that are well known to those skilled in the art, such as the exemplary methods provided herein.

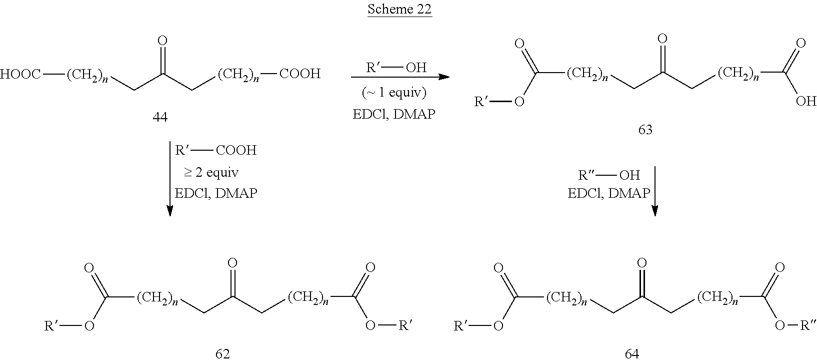

The synthesis of lipid 9 can be carried out as shown in Scheme 23. This synthetic sequence also exemplifies a method for the introduction of a type 1 ionizable head group. Thus, dihydroxyketone 47 is converted into diester 65 by reaction with excess acid 21 in the presence of a condensing agents such as EDCI and a tertiary amine base such as triethylamine, optionally in the presence of DMAP, as outlined earlier in Scheme 22. The ketone in 65 is selectively reduced with a suitable reducing agent, such as sodium borohydride, in an appropriate solvent, such as an alcohol, for instance, ethanol. Alcohol 66 thus produced is esterified with 4-(dimethylamino)butyric acid, or

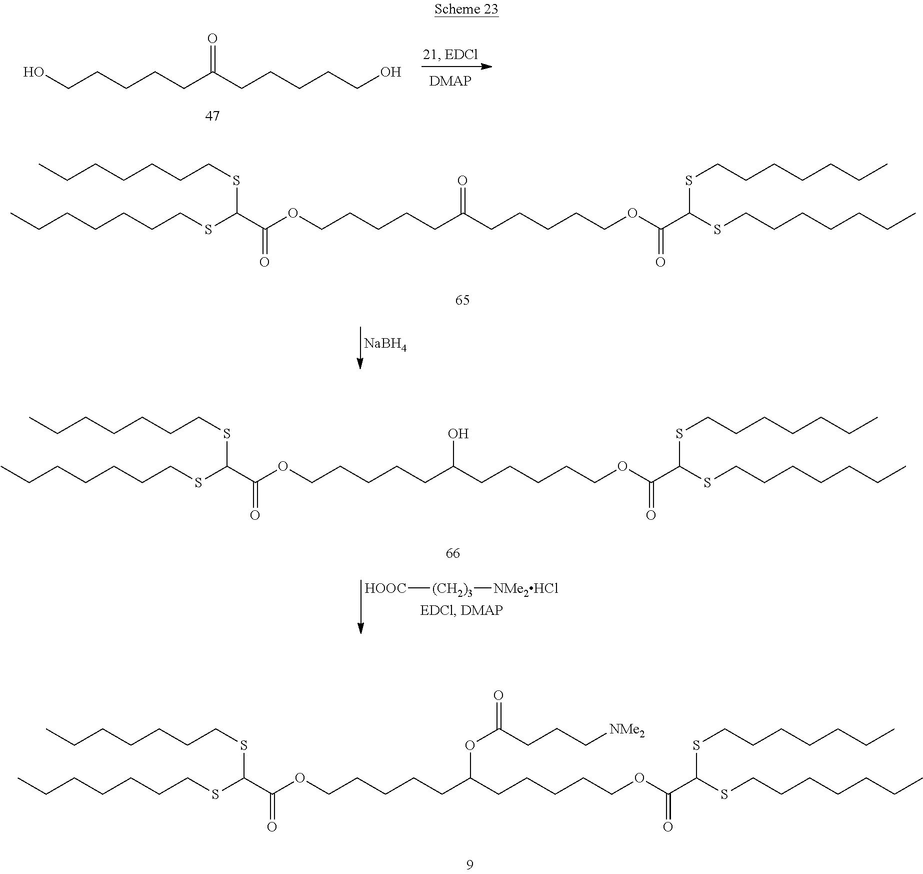

the corresponding hydrochloride salt, in the presence of a condensing agent such as EDCI and a tertiary amine base such as triethylamine, optionally in presence of DMAP, resulting in the introduction of a type 1 ionizable head group and in formation of lipid 9.

The synthesis of lipid 10 (Scheme 24) can be achieved in a similar way, except that acid 22 is used to esterify 47.

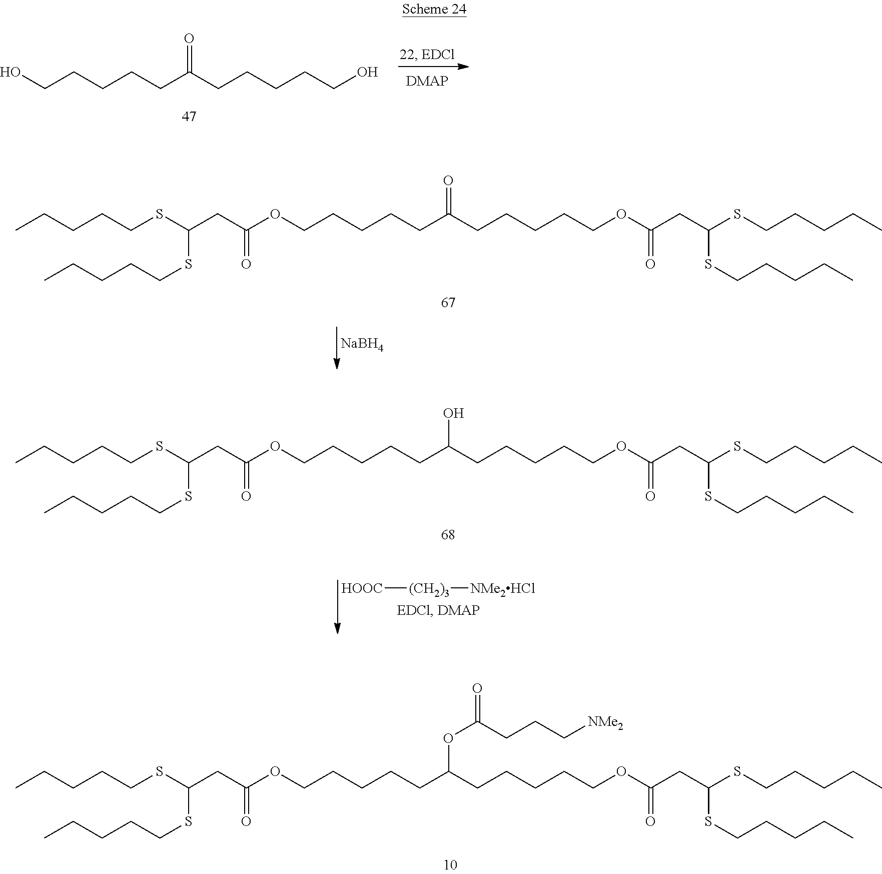

The synthesis of lipid 11 (Scheme 25) starts with the mono-esterification of 47 by reaction with about one equivalent of 2-hexyldecanoic acid in the presence of a condensing agents such as EDCI and a tertiary amine base such as triethylamine, optionally in the presence of DMAP, as outlined earlier in Scheme 21. The resulting 69 is further esterified with acid 20 in the presence of a condensing agents such as EDCI and a tertiary amine base such as triethylamine, optionally in the presence of DMAP, leading to the formation of ketone 70. The keto carbonyl group in 70 is then transformed into a type 1 ionizable head group by the method outlined in Scheme 24 above.

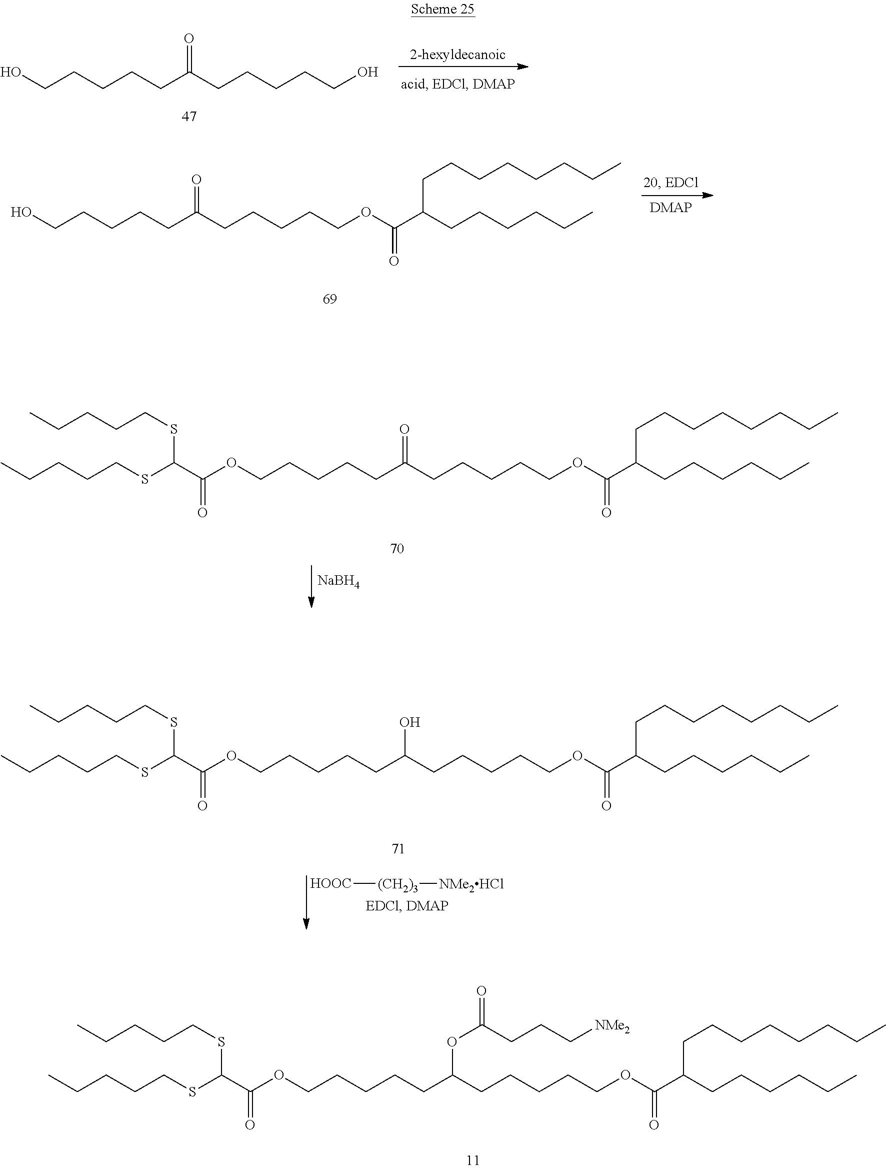

Lipid 12 can be prepared (Scheme 26) by esterification of alcohol 71 with 3-(dimethylamino)-propanoic acid or the corresponding hydrochloride salt by the method of Scheme 23 above.

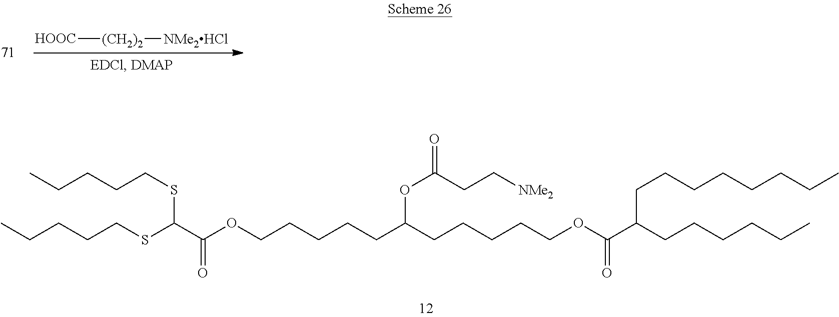

The synthesis of lipid 13 (Scheme 27) illustrates a method for the introduction of a type 2 ionizable head group. Thus, ketone 65 is converted into ketal 72 by reaction with 1,2,4-butanetriol and an acid catalyst, for example, pyridinium para-toluenesylfonate (PPTS), at elevated temperature and preferably with continuous azeotropic removal of water. For example, the reaction can be carried out in refluxing toluene using a Dean-Stark trap for water removal. The OH group in product 72 is transformed into a good leaving group, for example, sulfonate ester such as tosylate 73. Heating of 73 with dimethylamine in an appropriate solvent or mixture of solvents, optionally with microwave irradiation, produces lipid 13.

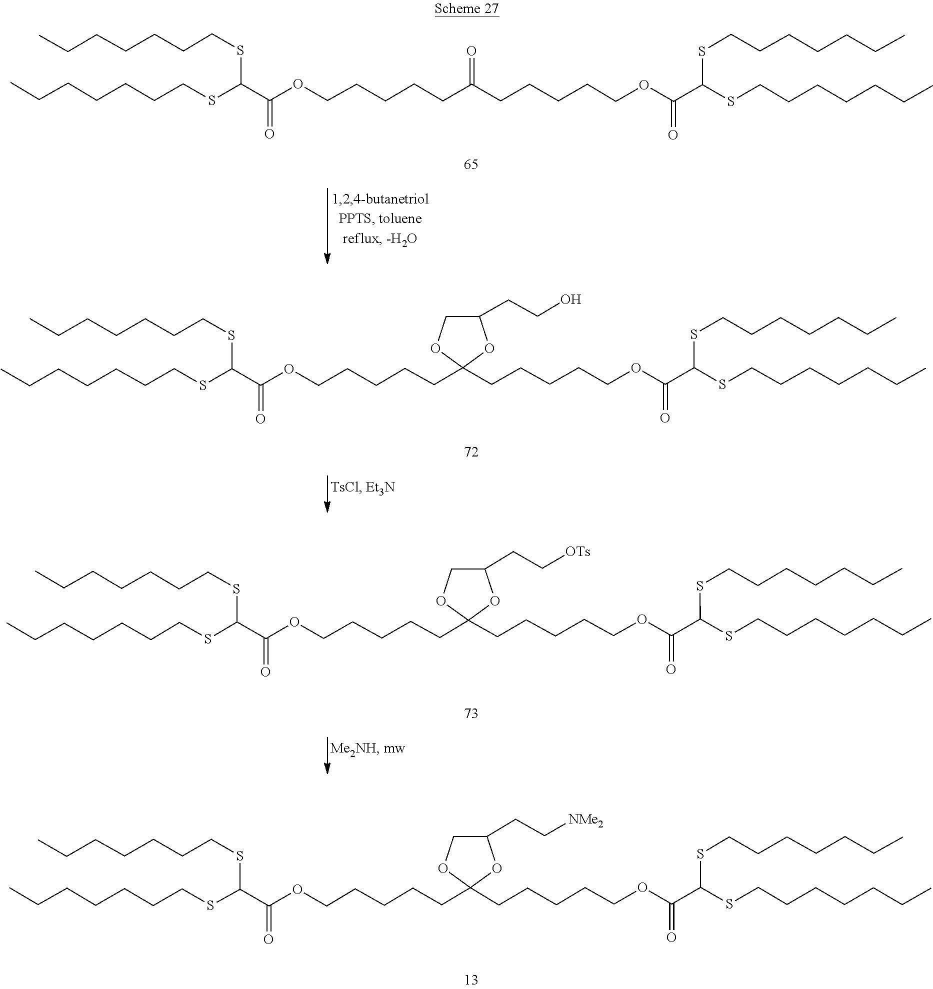

The synthesis of lipid 14 (Scheme 28) illustrates a method for the introduction of a type 3 ionizable head group. Thus, ketal 72 of Scheme 27 is esterified with 4-(dimethylamino)butyric acid, or the corresponding hydrochloride salt, by the method outlined in Scheme 23 above.

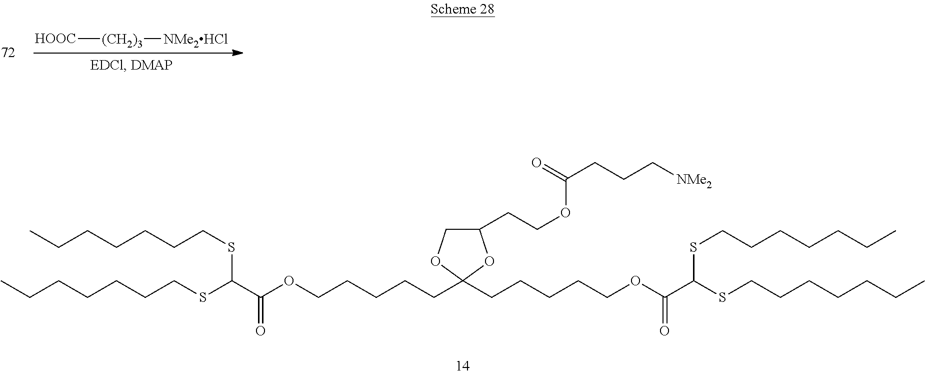

The synthesis of lipid 15 (Scheme 29) illustrates a method for the introduction of a type 7 ionizable head group. Thus, ketone 70 of Scheme 25 is reductively aminated with O-protected 4-amino-1-butanol 74 in an appropriate solvent, for example, 1,2-dichloroethane (DCE), in the presence of a reducing agent, for example, a borohydride reagent such as sodium triacetoxyborohydride, and optionally in the presence of an acid catalyst such as acetic acid. The resulting 75 is then reductively N-alkylated by reaction with an aldehyde and a suitable reducing agent in an appropriate solvent, optionally in the presence of an acid catalyst. For example, 75 can be transformed into 76 by reaction with aqueous formaldehyde in THF in the presence of NaBH(OAc)₃. Treatment of 76 with a source of fluoride ion, for example, pyridine-HF, releases the silyl protecting group to give 15.

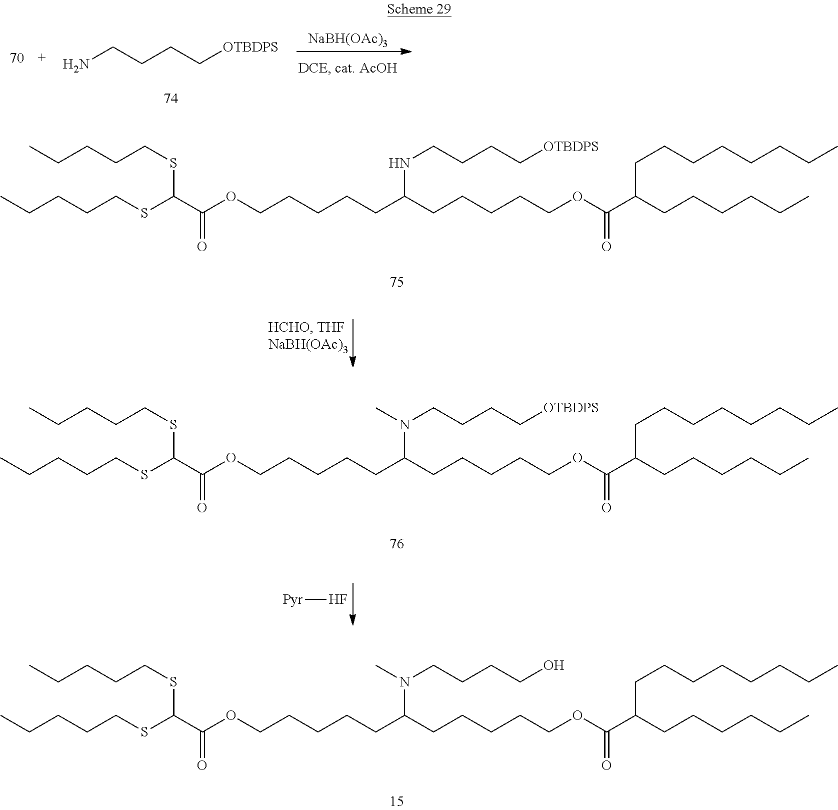

The synthesis of lipid 16 (Scheme 30) entails the esterification of compound 69 with acid 22, followed by conversion of the resulting 77 into lipid 16 by the method of Scheme 29.

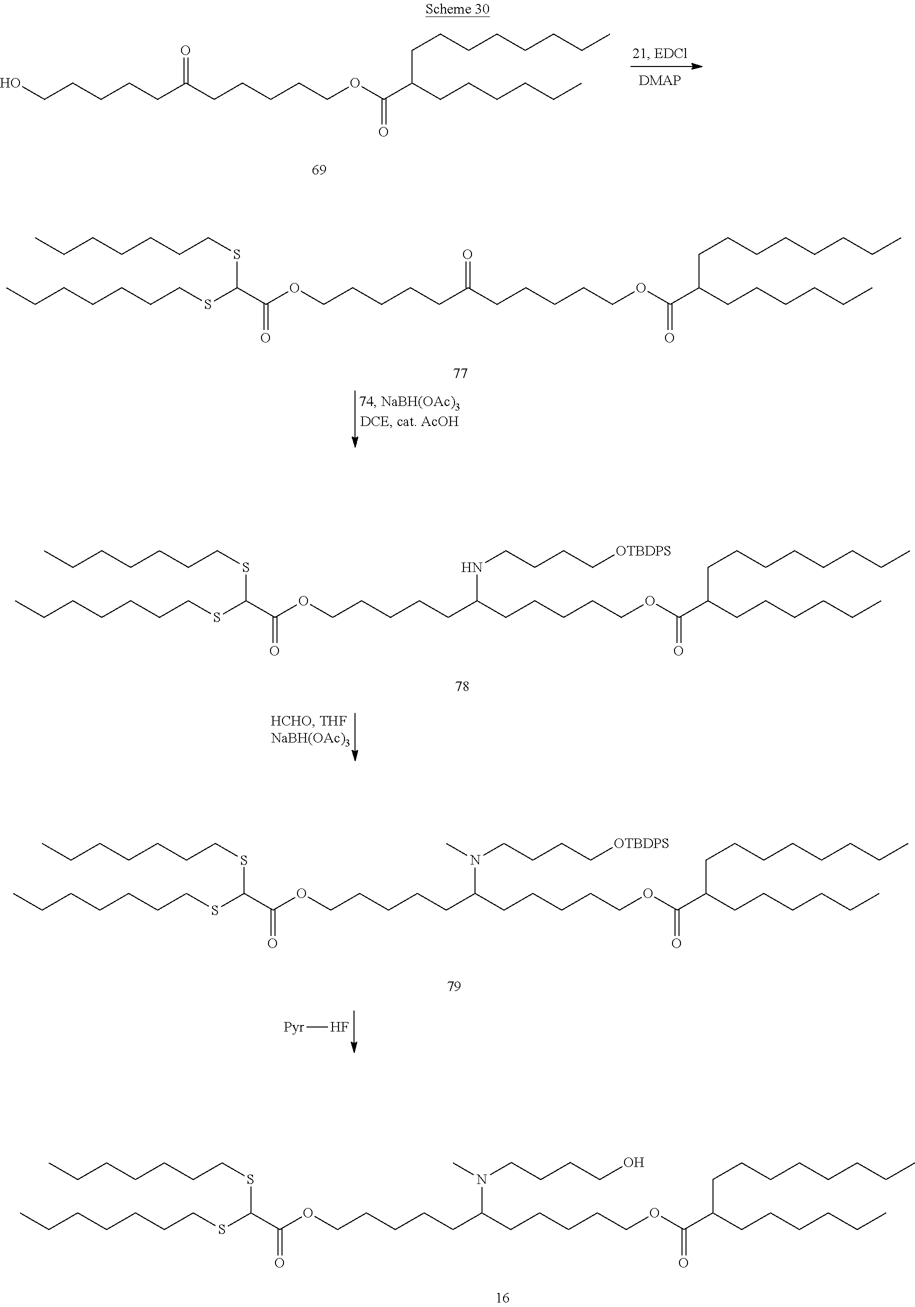

The synthesis of lipid 17 (Scheme 31) entails the esterification of compound 69 with acid 23, followed by conversion of the resulting 80 into lipid 16 by the method of Scheme 29.

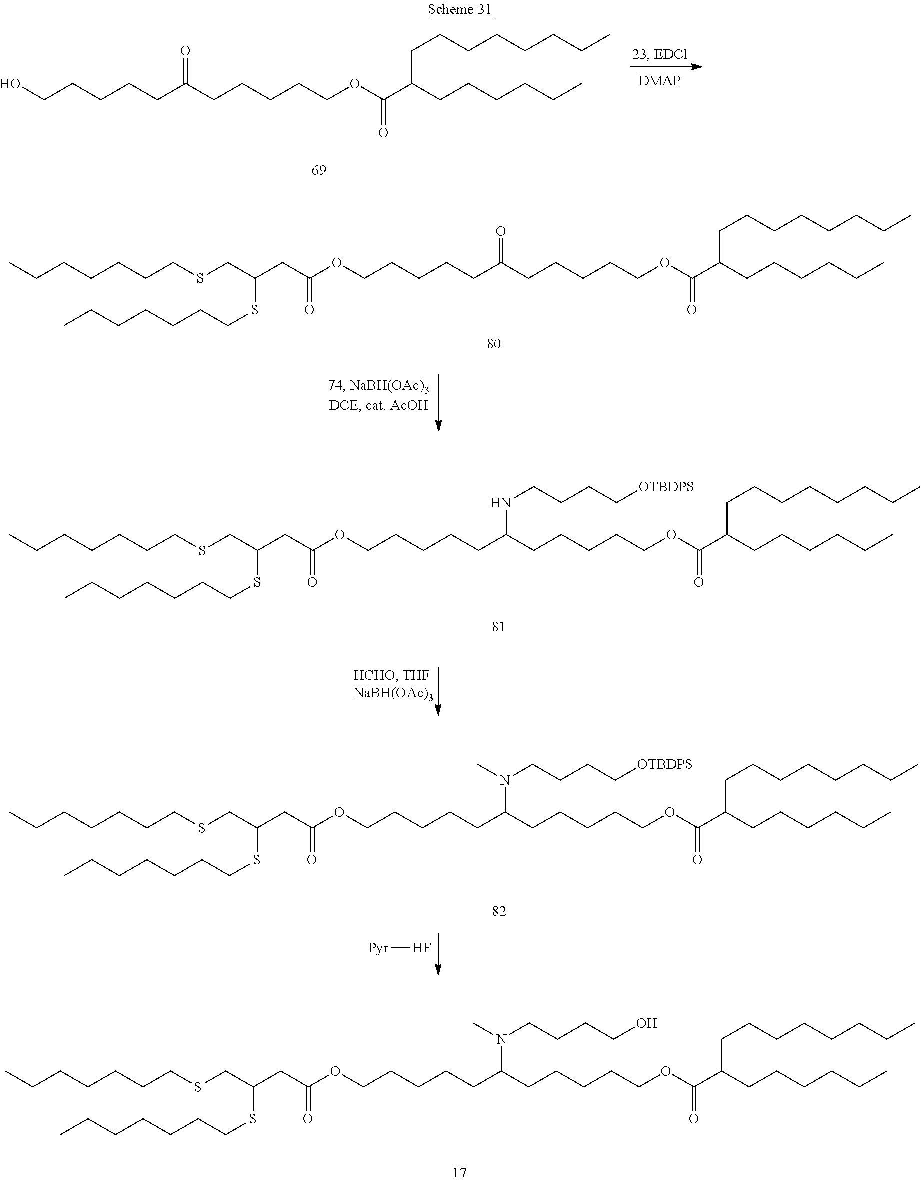

The synthesis of lipid 18 (Scheme 32) starts with the double esterification of ketodiacid 55 with alcohol 24 by the method shown earlier in Scheme 22. Accordingly, the reaction of 55 with at least 2 molar equivalents of 24 in the presence of a condensing agent, for example, a carbodiimide such as EDCI, and DMAP gives 83. This material can be converted into 18 by a sequence analogous to that outlined in Scheme 29 above, except that the first reductive amination step is carried out with amine 84, recognized as the O-TBDPS derivative of 3-amino-1-propanol. Substance 85 thus obtained is then subjected to reductive methylation (see compound 86) followed by release of the silyl group to produce lipid 18.

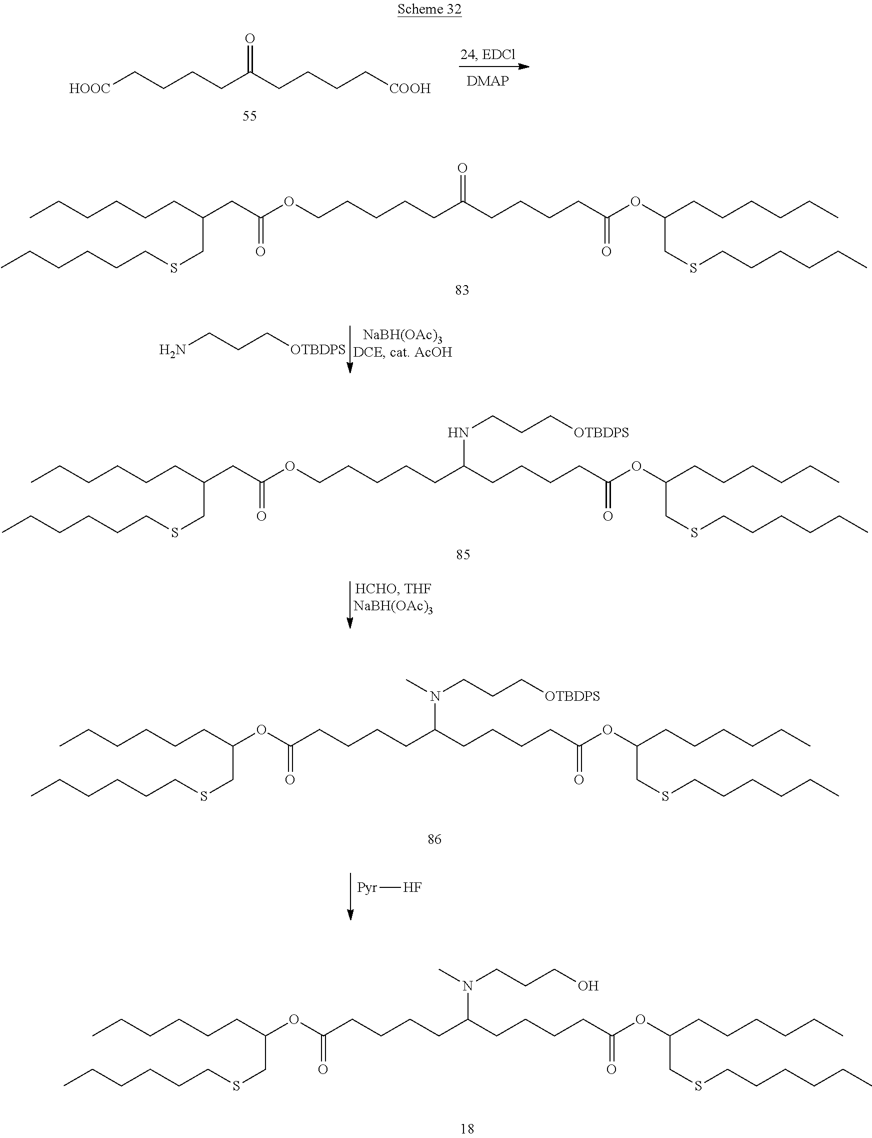

The synthesis of lipid 19 (Scheme 33) starts with the mono-esterification of ketodiacid 55 with 2-hexyl-1-decanol by the method outlined earlier in Scheme 22. Accordingly, the reaction of 55 with about 1 molar equivalent of 2-hexyl-1-decanol in the presence of a condensing agent, for example, a carbodiimide such as EDCI, and DMAP gives 87. Further esterification of 87 with alcohol 24 in the presence of a condensing agent, for example, a carbodiimide such as EDCI, and DMAP produces 88, which can be transformed into lipid 19 by the method shown earlier in Scheme 29.

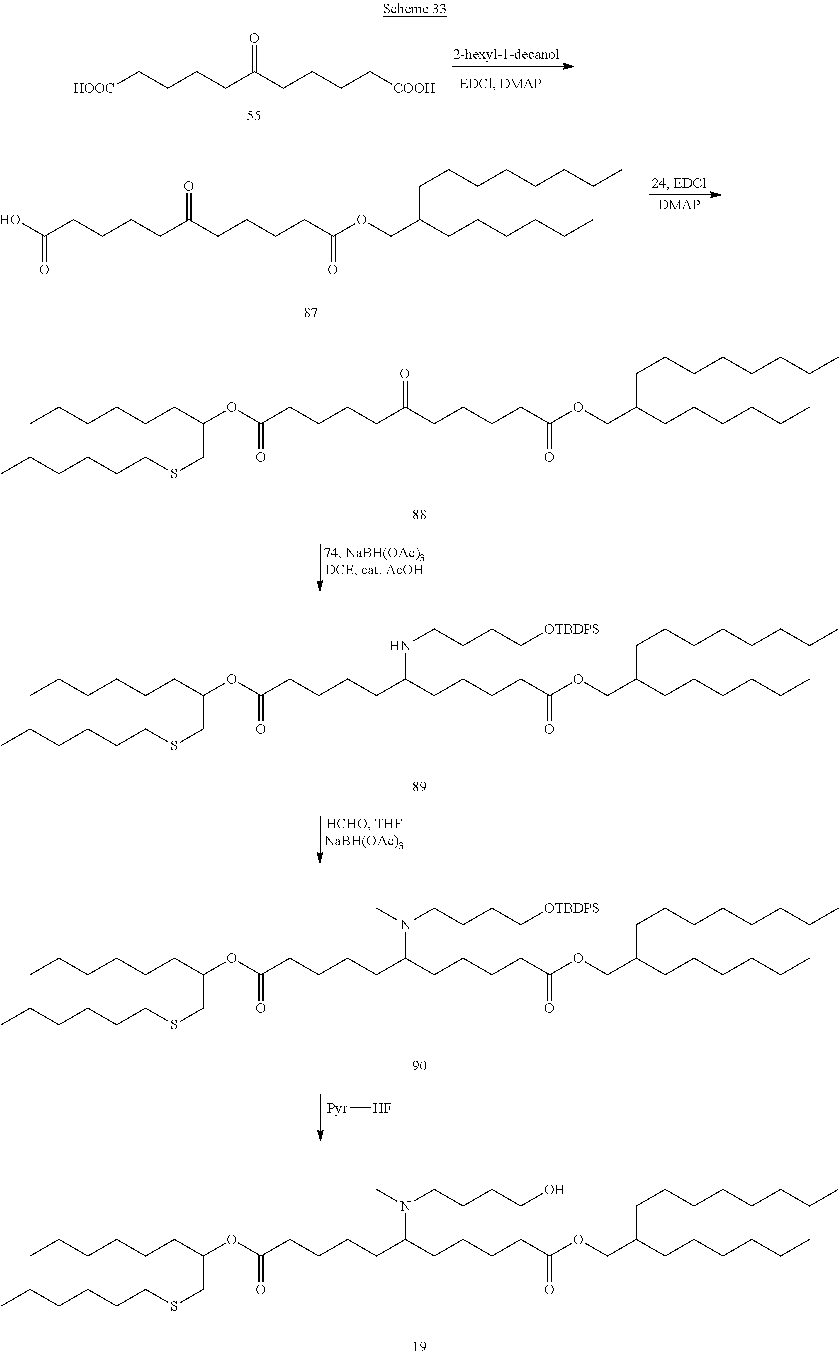

### Formulation of the Above Lipids in a Delivery Vehicle

The lipids of the disclosure may be formulated in a variety of drug delivery vehicles (also referred to herein as a “delivery vehicle”) known to those of ordinary skill in the art. An example of a delivery vehicle is a lipid nanoparticle, which includes liposomes, lipoplexes, polymer nanoparticles comprising lipids, polymer-based nanoparticles, emulsions, and micelles.

In one embodiment, a lipid having the structure of Formula A of the disclosure is formulated in a delivery vehicle by mixing the lipid with additional lipids, including helper lipids, such as vesicle forming lipids and optionally an aggregation inhibiting lipid, such as a hydrophilic polymer-lipid conjugate (e.g., PEG-lipid).

As set forth previously, a helper lipid includes a sterol, a diacylglycerol, a ceramide or derivatives thereof.

Examples of sterols include cholesterol, or a cholesterol derivative, such as cholestanol, cholestanone, cholestenone, coprostanol, cholesteryl-2′-hydroxyethyl ether, cholesteryl-4′-hydroxybutyl ether, beta-sitosterol, fucosterol, and the like.

Examples of diacylglycerols include dipalmitoylphosphatidylcholine (DPPC), distearoylphosphatidylcholine (DSPC), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), and mixtures thereof. In certain embodiments, the phospholipid is DPPC, DSPC, a DSPC-cholesterol conjugate or mixtures thereof. These lipids may be synthesized or obtained from natural sources, such as from egg. The DSPC-cholesterol conjugate is a lipid in which one of the acyl chains is substituted with a cholesterol moiety link to the head group by a succinate linker.

A suitable ceramide derivative is egg sphingomyelin or dihydrosphingomyelin.

Delivery vehicles incorporating the lipids of the disclosure can be prepared using a wide variety of well described formulation methodologies known to those of skill in the art, including but not limited to extrusion, ethanol injection and in-line mixing. In one embodiment, the preparation method is an in-line mixing technique in which aqueous and organic solutions are mixed using a rapid-mixing device as described in Kulkarni et al., 2018, ACS Nano, 12:4787 and Kulkarni et al., 2017, Nanoscale, 36:133347, each of which is incorporated herein by reference in its entirety.

The delivery vehicle can also be a nanoparticle that is a lipoplex that comprises a lipid core stabilized by a surfactant. Vesicle-forming lipids may be utilized as stabilizers. The lipid nanoparticle in another embodiment is a polymer-lipid hybrid system that comprises a polymer nanoparticle core surrounded by stabilizing lipid. Nanoparticles comprising lipids of the disclosure may alternatively be prepared from polymers without lipids. Such nanoparticles may comprise a concentrated core of a therapeutic agent that is surrounded by a polymeric shell or may have a solid or a liquid dispersed throughout a polymer matrix.

Lipids described herein can also be incorporated into emulsions, which are drug delivery vehicles that contain oil droplets or an oil core. An emulsion can be lipid-stabilized. For example, an emulsion may comprise an oil filled core stabilized by an emulsifying component such as a monolayer or bilayer of lipids.

Lipids described herein may be incorporated into a micelle. Micelles are self-assembling particles composed of amphipathic lipids or polymeric components that are utilized for the delivery of agents present in the hydrophobic core.

### Delivery of Nucleic Acid, Genetic Material, Proteins, Peptides or Other Charged Agents

Lipids disclosed herein may facilitate the incorporation of a compound or molecule (referred to herein also as “cargo” or “cargo molecule”) bearing a net negative or positive charge into the delivery vehicle and subsequent delivery to a target cell in vitro or in vivo.

In one embodiment, the cargo molecule is genetic material, such as a nucleic acid. The nucleic acid includes, without limitation, RNA, including small interfering RNA (siRNA), small nuclear RNA (snRNA), micro RNA (miRNA), messenger RNA (mRNA) or DNA such as vector DNA or linear DNA. The nucleic acid length can vary and can include nucleic acid of 5-50,000 nucleotides in length. The nucleic acid can be in any form, including single stranded DNA or RNA, double stranded DNA or RNA, or hybrids thereof. Single stranded nucleic acid includes antisense oligonucleotides.

In one embodiment, the cargo is an mRNA, which includes a polynucleotide that encodes at least one peptide, polypeptide or protein. The mRNA includes, but is not limited to, small activating RNA (saRNA) and trans-amplifying RNA (taRNA), as described in co-pending WO 2022/251953A1, which is incorporated herein by reference.

The mRNA as used herein encompasses both modified and unmodified mRNA. In one embodiment, the mRNA comprises one or more coding and non-coding regions. The mRNA can be purified from natural sources, produced using recombinant expression systems and optionally purified, or may be chemically synthesized.

In those embodiments in which an mRNA is a chemically synthesized molecule, the mRNA can comprise nucleoside analogs such as analogs having chemically modified bases or sugars, and/or backbone modifications. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, 2-thiocytidine, pseudouridine, and 5-methylcytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The mRNAs of the disclosure may be synthesized according to any of a variety of known methods. For example, mRNAs in certain embodiments may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor.

In some embodiments, in vitro synthesized mRNA may be purified before encapsulation to remove undesirable impurities including various enzymes and other reagents used during mRNA synthesis.

The present disclosure may be used to encapsulate mRNAs of a variety of lengths. In some embodiments, the present disclosure may be used to encapsulate in vitro synthesized mRNA ranging from about 1-20 kb, about 1-15 kb, about 1-10 kb, about 5-20 kb, about 5-15 kb, about 5-12 kb, about 5-10 kb, about 8-20 kb, or about 8-15 kb in length.

Typically, mRNA synthesis includes the addition of a “cap” on the 5′ end, and a “tail” on the 3′ end. The cap can provide resistance to nucleases found in most eukaryotic cells. The “tail” can be incorporated into the 3′ end to protect the mRNA from exonuclease degradation.

In some embodiments, mRNAs include a 5′ and/or 3′ untranslated region. In some embodiments, a 5′ untranslated region includes one or more elements that affect the stability or translation of mRNA, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer.

In a further embodiment, the mRNA is circular. Advantageously, such mRNA lacks 5′ and 3′ ends and thus may be more stable in vivo due to its resistance to degradation by exonucleases. The circular mRNA may be prepared by any known method, including any one of the methods described in Deviatkin et al., 2023, Vaccines, 11(2), 238, which is incorporated herein by reference. Translation of the circular mRNA is carried out by a cap-independent initiation mechanism.

While mRNA provided from in vitro transcription reactions may be desirable in certain embodiments, other sources of mRNA are contemplated, such as mRNA produced from bacteria, fungi, plants, and/or animals.

The mRNA sequence may comprise a reporter gene sequence, although the inclusion of a reporter gene sequence in pharmaceutical formulations for administration is optional. Such sequences may be incorporated into mRNA for in vitro studies or for in vivo studies in animal models to assess biodistribution.

In another embodiment, the cargo is an siRNA. An siRNA becomes incorporated into endogenous cellular machineries to result in mRNA breakdown, thereby preventing transcription. Since RNA is easily degraded, its incorporation into a delivery vehicle can reduce or prevent such degradation, thereby facilitating delivery to a target site.

The siRNA encompassed by embodiments of the disclosure may be used to specifically inhibit expression of a wide variety of target polynucleotides. The siRNA molecules targeting specific polynucleotides may be readily prepared according to procedures known in the art. An siRNA target site may be selected and corresponding siRNAs may be chemically synthesized, created by in vitro transcription, or expressed from a vector or PCR product. A wide variety of different siRNA molecules may be used to target a specific gene or transcript. The siRNA may be double-stranded RNA, or a hybrid molecule comprising both RNA and DNA, e.g., one RNA strand and one DNA strand. The siRNA may be of a variety of lengths, such as 15 to 30 nucleotides in length or 20 to 25 nucleotides in length. In certain embodiments, the siRNA is double-stranded and has 3′ overhangs or 5′ overhangs. In certain embodiments, the overhangs are UU or dTdT 3′. In particular embodiments, the siRNA comprises a stem loop structure.

In a further embodiment, the cargo molecule is a microRNA or small nuclear RNA.

Micro RNAs (miRNAs) are short, noncoding RNA molecules that are transcribed from genomic DNA, but are not translated into protein. These RNA molecules are believed to play a role in regulation of gene expression by binding to regions of target mRNA. Binding of miRNA to target mRNA may downregulate gene expression, such as by inducing translational repression, deadenylation or degradation of target mRNA. Small nuclear RNA (snRNA) are typically longer noncoding RNA molecules that are involved in gene splicing. The snRNA molecules may have therapeutic importance in diseases that are an outcome of splicing defects.

In another embodiment, the cargo is a DNA vector as described in co-owned and co-pending WO2022/251959, which is incorporated herein by reference. The DNA vector may be administered to a subject for the purpose of repairing, enhancing or blocking or reducing the expression of a cellular protein or peptide. Accordingly, the nucleotide polymers can be nucleotide sequences including genomic DNA, cDNA, or RNA.

As will be appreciated by those of skill in the art, the vectors may encode promoter regions, operator regions or structural regions. The DNA vectors may contain double-stranded DNA or may be composed of a DNA-RNA hybrid. Non-limiting examples of double-stranded DNA include structural genes, genes including operator control and termination regions, and self-replicating systems such as vector DNA.

Single-stranded nucleic acids include antisense oligonucleotides (complementary to DNA and RNA), ribozymes and triplex-forming oligonucleotides. In order to have prolonged activity, the single-stranded nucleic acids will preferably have some or all of the nucleotide linkages substituted with stable, non-phosphodiester linkages, including, for example, phosphorothioate, phosphorodithioate, phophoroselenate, or O-alkyl phosphotriester linkages.

The DNA vectors may include nucleic acids in which modifications have been made in one or more sugar moieties and/or in one or more of the pyrimidine or purine bases. Such sugar modifications may include replacement of one or more hydroxyl groups with halogens, alkyl groups, amines, azido groups or functionalized as ethers or esters. In another embodiment, the entire sugar may be replaced with sterically and electronically similar structures, including aza-sugars and carbocyclic sugar analogs. Modifications in the purine or pyrimidine base moiety include, for example, alkylated purines and pyrimidines, acylated purines or pyrimidines, or other heterocyclic substitutes known to those of skill in the art.

The DNA vector may be modified in certain embodiments with a modifier molecule such as a peptide, protein, steroid or sugar moiety. Modification of a DNA vector with such molecule may facilitate delivery to a target site of interest. In some embodiments, such modification translocates the DNA vector across a nucleus of a target cell. By way of example, a modifier may be able to bind to a specific part of the DNA vector (typically not encoding of the gene-of-interest), but also has a peptide or other modifier that has nucleus-homing effects, such as a nuclear localization signal. A non-limiting example of a modifier is a steroid-peptide nucleic acid conjugate as described by Rebuffat et al., 2002, Faseb J. 16(11):1426-8, which is incorporated herein by reference. The DNA vector may contain sequences encoding different proteins or peptides. Promoter, enhancer, stress or chemically-regulated promoters, antibiotic-sensitive or nutrient-sensitive regions, as well as therapeutic protein encoding sequences, may be included as required. Non-encoding sequences may be present as well in the DNA vector.

The nucleic acids used in the present method can be isolated from natural sources, obtained from such sources as ATCC or GenBank libraries or prepared by synthetic methods.

Synthetic nucleic acids can be prepared by a variety of solution or solid phase methods.

Generally, solid phase synthesis is preferred. Detailed descriptions of the procedures for solid phase synthesis of nucleic acids by phosphite-triester, phosphotriester, and H-phosphonate chemistries are widely available.

In one embodiment, the DNA vector is double stranded DNA and comprises more than 700 base pairs, more than 800 base pairs or more than 900 base pairs or more than 1000 base pairs.

In another embodiment, the DNA vector is a nanoplasmid or a minicircle.

Gene editing systems can also be incorporated into delivery vehicles comprising the charged lipid. This includes a Cas9-CRISPR, TALEN and zinc finger nuclease gene editing system. In the case of Cas9-CRISPR, a guide RNA (gRNA), together with a plasmid or mRNA encoding the Cas9 protein may be incorporated into a delivery vehicle comprising the lipids described herein. Optionally, a ribonucleoprotein complex may be incorporated into a delivery vehicle comprising the lipid described herein. Likewise, the disclosure includes embodiments in which genetic material encoding DNA binding and cleavage domains of a zinc finger nuclease or TALEN system are incorporated into a delivery vehicle together with the lipids of the disclosure.

While a variety of nucleic acid cargo molecules are described above, it will be understood that the above examples are non-limiting and the disclosure is not to be considered limiting with respect to the particular cargo molecule encapsulated in the delivery vehicle.

For example, the lipids described herein may also facilitate the incorporation of proteins and peptides into a delivery vehicle, which includes ribonucleoproteins. This includes both linear and non-linear peptides, proteins or ribonucleoproteins.

While pharmaceutical compositions are described above, the lipids described herein can be a component of any nutritional, cosmetic, cleaning or foodstuff product.

### Pharmaceutical Formulations

The ionizable lipids of the disclosure may be present in a salt form. The salt is typically a pharmaceutically acceptable salt. Salts derived from inorganic bases include aluminum, ammonium, calcium, copper, ferric, ferrous, lithium, magnesium, manganic salts, manganous, potassium, sodium, and zinc. In one embodiment, the base is selected from ammonium, calcium, magnesium, potassium and sodium. Salts derived from pharmaceutically acceptable organic non-toxic bases include salts of primary, secondary and tertiary amines, substituted amines including naturally occurring substituted amines, cyclic amines and the like.

In some embodiments, the delivery vehicle comprising the cargo molecule is part of a pharmaceutical composition and is administered to treat and/or prevent a disease condition. The treatment may provide a prophylactic (preventive), ameliorative or a therapeutic benefit. The pharmaceutical composition will be administered at any suitable dosage.

In one embodiment, the pharmaceutical compositions is administered parentally, i.e., intra-arterially, intravenously, subcutaneously or intramuscularly. In yet a further embodiment, the pharmaceutical compositions are for intra-tumoral or in-utero administration. In another embodiment, the pharmaceutical compositions are administered intranasally, intravitreally, subretinally, intrathecally or via other local routes.

The pharmaceutical composition comprises pharmaceutically acceptable salts and/or excipients.

The compositions described herein may be administered to a patient. The term patient as used herein includes a human or a non-human subject.

The following examples are given for the purpose of illustration only and not by way of limitation on the scope of the invention.

### EXAMPLES

### Materials

The lipid 1,2-distearoyl-sn-glycero-3-phosphorylcholine (DSPC) and 1,2-dimyristoyl-rac-glycero-3-methoxypolyethylene glycol-2000 (PEG-DMG) were purchased from Avanti Polar Lipids (Alabaster, AL). Cholesterol and 10× Phosphate Buffered Saline (pH 7.4) were purchased from Sigma Aldrich (St Louis, MO). The ionizable amino-lipid was synthesized as previously described in WO 2022/246555, which is incorporated herein by reference.

An mRNA encoding firefly luciferase purchased from RNA Technologies and Therapeutics (Montreal, QC) was used to analyse luciferase activity.

### Methods

Preparation of Lipid Nanoparticles (LNP) Containing mRNA or siRNA

Lipids 1 or 2 described herein, DSPC, cholesterol, and PEG-DMG, were dissolved in ethanol at the appropriate ratios to a final concentration of 10 mM total lipid. Nucleic acid (siRNA or mRNA) was dissolved in an appropriate buffer such as 25 mM sodium acetate pH 4 or sodium citrate pH 4 to a concentration necessary to achieve the appropriate amine-to-phosphate ratios. The aqueous and organic solutions were mixed using a rapid-mixing device as described in Kulkarni et al., 2018, ACS Nano, 12:4787 and Kulkarni et al., 2017, Nanoscale, 36:133347 (each incorporated herein by reference) at a flow rate ratio of 3:1 (v/v; respectively) and a total flow rate of 20 mL/min. The resultant mixture was dialyzed directly against 1000-fold volume of PBS pH 7.4. All formulations were concentrated using an Amicon centrifugal filter unit and analysed using the methods described below.

### Analysis of LNP

Particle size analysis of LNPs in PBS was carried out using backscatter measurements of dynamic light scattering with a Malvern Zetasizer™ (Worcestershire, UK). The reported particle sizes correspond to the number-weighted average diameters (nm). Total lipid concentrations were determined by extrapolation from the cholesterol content, which was measured using the Cholesterol E-Total Cholesterol Assay™ (Wako Diagnostics, Richmond, VA) as per the manufacturer's recommendations. Encapsulation efficiency of the formulations was determined using the Quant-iT RiboGreen™ Assay kit (Invitrogen, Waltham, MA). Briefly, the total siRNA or mRNA content in solution was measured by lysing lipid nanoparticles in a solution of TE containing 2% Triton Tx-100, and free DNA vector in solution (external to LNP) was measured based on the RiboGreen fluorescence in a TE solution without Triton. Total siRNA or mRNA content in the formulation was determined using a modified Bligh-Dyer extraction procedure. Briefly, LNP formulations containing siRNA or mRNA were dissolved in a mixture of chloroform, methanol, and PBS that results in a single phase and the absorbance at 260 nm measured using a spectrophotometer.

### In Vivo Analysis in CD-1 Mice

LNP-mRNA encoding firefly luciferase were injected intravenously (tail-vein) into 6-8 week old CD-1 mice. Four hours following injection, the animals were euthanized and the liver and spleen and isolated. Tissue was homogenized in Glo Lysis™ buffer and a luciferase assay performed using the Steady Glo™ Luciferase assay kit (as per manufacturers recommendations).

### Organic Synthesis of Lipids 5-19.

Unless otherwise specified, all reagents and solvents were commercial products and were used without further purification, except THF (freshly distilled from Na/benzophenone under Ar), CH₂Cl₂ (freshly distilled from CaH₂ under Ar). “Dry methanol” was freshly distilled from magnesium turnings. All reactions were performed under an argon atmosphere. Reaction mixture from aqueous workups were dried by passing over a plug of anhydrous Na₂SO₄ held in a filter tube and concentrated under reduced pressure on a rotary evaporator. Thin-layer chromatography was performed on silica gel plates coated with silica gel (Merck 60 F254 plates) and column chromatography was performed on 230-400 mesh silica gel. Visualization of the developed chromatogram was performed by staining with I₂ or potassium permanganate solution. ¹H and ¹³C nuclear magnetic resonance (NMR) spectra were recorded at room temperature in CDCl₃ solutions. ¹H NMR spectra were referenced to residual CHCl₃ (7.26 ppm) and ¹³C NMR spectra were referenced to the central line of the CDCl₃ triplet (77.00 ppm). Chemical shifts are reported in parts per million (ppm) on the δ scale. Multiplicities are reported as “s” (singlet), “d” (doublet), “t” (triplet), “q” (quartet), “m” (multiplet), and further qualified as “app” (apparent) and “br” (broad). Low- and high-resolution mass spectra (m/z) were obtained in the electrospray (ESI) and field desorption/field ionisation (FD/FI) mode.

The synthesis of lipids 5-6 from 4-amino-1-butanol was carried out as set forth below. As discussed, the synthesis of said lipids involves subjecting 4-amino-1-butanol mono-N-alkylation or di-N-alkylation with certain alkyl halides or sulfonates under appropriate conditions. This technology is as set forth in co-owned and co-pending WO 2023/173203 (incorporated herein by reference).

The synthesis of lipids 7-17 from caprolactone was carried out as set forth below. As discussed, the synthesis of said lipids involves subjecting certain esters or lactones to Claisen condensation under Mukaiyama conditions. This technology is as set forth in co-owned and co-pending WO 2023/147657 (incorporated herein by reference). The products of such Claisen reactions are subsequently converted into the final products as outlined in the Schemes above and as described below.

### Example 1: Methods for Chemically Synthesizing Ionizable Lipids 5-19

### (A) Preparation of Building Blocks

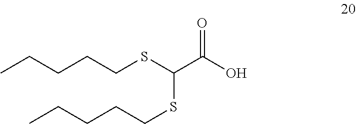 [SMILES: CCCCCSC(SCCCCC)C(=O)O]

(i) 2,2-bis(Pentylthio)acetic acid (20). A solution of glyoxylic acid monohydrate (1.00 g, 10.9 mmol), 1-pentanethiol (2.97 mL, 23.9 mmol) and TsOH (188 mg, 1.09 mmol) in toluene (40.0 mL) was refluxed under inert atmosphere for 5 hours. The mixture was then cooled to room temperature and washed with water (30.0 mL) and brine (30.0 mL). The organic layer was dried (Na₂SO₄) and concentrated to yield 41 (2.80 g, quantitative) which was used in the next step without further purification. ¹H NMR (400 MHz, CDCl₃) δ 4.37 (s, 1H), 2.82-2.61 (m, 4H), 1.62 (p, J=7.4 Hz, 4H), 1.47-1.27 (m, 8H), 0.90 (t, J=7.0 Hz, 6H).

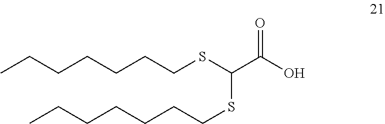 [SMILES: CCCCCCCSC(SCCCCCCC)C(=O)O]

(ii) 2,2-bis(Peptylthio)acetic acid (21). Prepared by procedure (i) above but with 1-heptanethiol in lieu of 1-pentanethiol. ¹H NMR (400 MHz, CDCl₃) δ 4.34 (s, 1H), 2.83-2.64 (m, 4H), 1.61 (p, J=7.3 Hz, 4H), 1.47-1.19 (m, 16H), 0.87 (t, 6H).

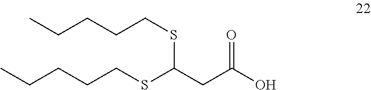 [SMILES: CCCCCSC(CC(=O)O)SCCCCC]

(iii) 3,3-bis(Pentylthio)propanoic acid (22). To a solution of methyl 3,3-dimethoxypropanoate (300 mg, 2.02 mmol) and 1-pentanethiol (0.551 mL, 4.44 mmol) in 1,4-dioxane (10.0 mL) was added conc. H₂SO₄ (0.025 mL). The resulting mixture was refluxed for 4 hours under inert atmosphere, cooled to room temperature, diluted with water (20.0 mL) and extracted with CH₂Cl₂ (3×20.0 mL). The combined organics were dried (Na₂SO₄) and concentrated to yield the methyl 3,3-bis(pentylthio)propanoate (560 mg, crude) which was used in the next step without further purification. ¹H NMR (400 MHz, CDCl₃) δ 4.21 (t, J=7.6 Hz, 1H), 3.71 (s, 3H), 2.83-2.76 (m, 2H), 2.71-2.48 (m, 4H), 1.75-1.53 (m, 4H), 1.46-1.26 (m, 8H), 0.97-0.83 (m, 6H). To a solution of this material (560 mg, crude) in EtOH (10.0 mL) was added 3 N NaOH (3.00 mL). The resulting mixture was stirred for 5 hours at room temperature, acidified to pH 2 with conc. HCl, diluted with water (10.0 mL) and extracted with CH₂Cl₂ (3×15.0 mL) to yield the acid 22 which was used directly in the next step without further purification (476 mg, crude). ¹H NMR (400 MHz, CDCl₃) δ 4.23 (t, J=7.5 Hz, 1H), 2.91-2.81 (m, 2H), 2.75-2.56 (m, 4H), 1.80 1.55 (m, 4H), 1.50-1.24 (m, 8H), 0.99-0.87 (m, 6H).

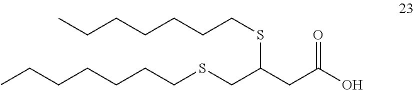 [SMILES: CCCCCCCSCC(CC(=O)O)SCCCCCCC]

(iv) 3,4-bis(Heptylthio)butanoic acid (23). A solution of methyl 4-bromocrotonate (500 mg, 2.79 mmol), 1-heptanethiol (1.10 mL, 6.98 mmol) and K₂CO₃ (1.16 g, 8.37 mmol) in DMF (12.0 mL) was heated at 70° C. for 18 hours under inert atmosphere then cooled to room temperature. The mixture was diluted with water (30.0 mL) and extracted with toluene (3×25 mL). The combined organics were washed (brine), dried (Na₂SO₄) and concentrated. The residue was purified by silica chromatography (0-25% EtOAc in Hexanes) to yield the methyl 3,4-bis(heptylthio)butanoate as an oil (997 mg, quantitative). ¹H NMR (400 MHz, CDCl₃) δ 3.70 (s, 3H), 3.18 (ddt, J=9.3, 8.3, 5.2 Hz, 1H), 3.05-2.83 (m, 2H), 2.72-2.45 (m, 6H), 1.57 (tt, J=7.8, 4.3 Hz, 4H), 1.42-1.22 (m, 16H), 0.94-0.81 (m, 6H). This material (997 mg) was converted into 23 by the saponification method of procedure (iii). ¹H NMR (400 MHz, CDCl₃) δ 3.20-3.12 (m, 1H), 3.04 (dd, J=16.3, 5.3 Hz, 1H), 2.93 (dd, J=13.6, 4.8 Hz, 1H), 2.71-2.49 (m, 6H), 1.68-1.52 (m, 4H), 1.46-1.17 (m, 16H), 0.95-0.82 (m, 6H).

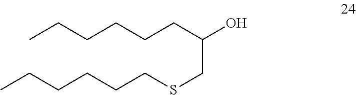 [SMILES: CCCCCCSCC(O)CCCCCC]

(v) 1-(Hexylthio)octan-2-ol (24). To a solution of 1,2-epoxyoctane (5.0 g, 39.0 mmol) and 1-hexanethiol (5.06 g, 42.8 mmol)) in ethanol (30.0 mL) was added NaOH (3.1 g, 78.0 mmol). The resulting mixture was stirred for 2 hours under nitrogen atmosphere at room temperature, then it was diluted with water (20.0 mL) and extracted with hexanes (3×20.0 mL). The combined extracts were washed with brine and concentrated. The residue was purified by silica chromatography (10% EtOAc in Hexanes) to yield the compound 24 as a yellowish oil (7.74 g, 80%). ¹H NMR (400 MHz, CDCl₃) δ 3.66-3.56 (m, 1H), 2.78-2.35 (m, 5H), 1.61-1.50 (m, 2H), 1.50-1.40 (m, 2H), 1.40-1.20 (m, 14H), 0.86 (t, J=6.91, 6H).

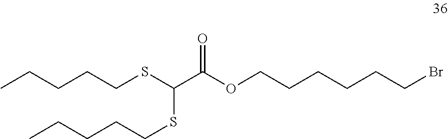 [SMILES: CCCCCSC(SCCCCC)C(=O)OCCCCCCBr]

(vi) 6-Bromohexyl 2,2-bis(pentylthio)acetate (36). A solution of 20 (650 mg, 2.48 mmol), 6-bromo-1-hexanol (584 mg, 3.22 mmol), EDCI-HCl (665 mg, 3.47 mmol) and DMAP (424 mg, 3.47 mmol) in CH₂Cl₂ (10.0 mL) was stirred under inert atmosphere for 18 hours then concentrated. The residue was purified by silica chromatography (0-20% EtOAc in Hexanes) to yield 36 (778 mg, 74%) as an oil. ¹H NMR (400 MHz, CDCl₃) δ 4.32 (s, 1H), 4.16 (t, J=6.6 Hz, 2H), 3.41 (t, J=6.8 Hz, 2H), 2.79-2.58 (m, 4H), 1.95-1.82 (m, 2H), 1.78-1.23 (m, 18H), 0.89 (t, J=7.1 Hz, 6H).

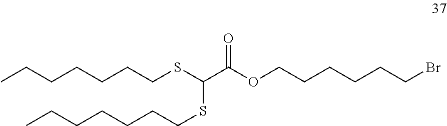 [SMILES: CCCCCCCSC(SCCCCCCC)C(=O)OCCCCCCBr]

(vii) 6-Bromohexyl 2,2-bis(heptylthio)acetate (37). Prepared from 21 by the same procedure employed for 36. ¹H NMR (400 MHz, CDCl₃) δ 4.30 (s, 1H), 4.13 (t, J=6.7 Hz, 2H), 3.60-3.53 (m, 2H), 2.77-2.54 (m, 8H), 1.74-1.44 (m, 12H), 1.44-1.19 (m, 20H), 0.91-0.82 (m, 6H).

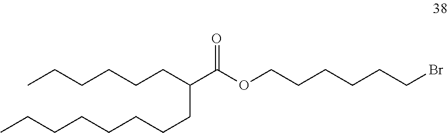 [SMILES: CCCCCCCCC(CCCCCC)C(=O)OCCCCCCBr]

(viii) 6-Bromohexyl 2-hexyldecanoate (38), Two drops of 98% sulfuric acid were added to solution of 2-hexyldecanoic acid (15.6 g, 60.8 mmol, 1.1 equiv) and 6-bromohexanol (10 g, 55.2 mmol, 1 equiv) in cyclohexane (30 mL), and the mixture was brought to reflux with continuous removal of water (Dean-Stark trap). After 24 h, some starting alcohol (ca. 10% by 1H NMR) was still present. One drop of 98% H₂SO₄ was added and the mixture was refluxed for another 24 h, whereupon the reaction was complete (¹H NMR). The reaction mixture was cooled to room temperature, diluted with hexanes (20 mL), washed with 5% aqueous Na₂CO₃ solution (2×10 mL), dried (Na₂SO₄) and evaporated. The crude product thus obtained (22 g, 97%) was used directly in the next step. ¹H NMR (400 MHz, CDCl₃) δ 4.10 (t, 2H, J=6.5), 3.40 (t, 2H, J=6.8), 2.31 (m, 1H), 1.87 (m, 2H), 1.8-1.4 (m, 30H), 0.88 (t, 6H, J=6.2 Hx). ¹³C NMR (100 MHz, CDCl₃) δ 176.8, 63.8, 45.9 33.6, 32.7, 32.5 (2 peaks), 31.9, 31.7, 29.6, 29.5, 29.2 (2 peaks), 28.5, 27.8, 27.5, 27.4, 25.2, 22.6 (2 peaks), 14.1 (2 peaks). LRMS: m/z 419 [M+H]⁺.

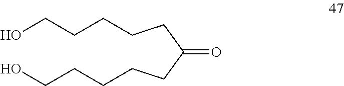 [SMILES: O=C(CCCCCO)CCCCCO]

(ix) 1,11-Dihydroxyundecan-6-one (47). A solution of TiCl₄ (74.8 g, 44 mL, 0.39 mol) in CH₂Cl₂ (200 mL) was added dropwise to a cold (0° C., ice bath), stirred solution of caprolactone (30 g, 0.26 mol) and triethylamine (Et₃N) (47.2 g, 67 mL, 0.46 mol) in CH₂Cl₂ (450 mL). After completion of the reaction, water (40 mL) was added and all volatiles were removed on rotatory evaporator (bath temperature 60° C.). The residue was extracted with several portions of 10% MeOH in CH₂Cl₂, and the combined extracts were dried over anhydrous Na₂SO₄. Evaporation of the solvent resulted in a yellowish oily residue, which was purified by column chromatography on silica gel (230-400 mesh) by eluting with a gradient of 5→7% MeOH in CH₂Cl₂. This provided 17.1 g of product 29 (65% yield) as an a off white solid. ¹H NMR (400 MHz, CDCl₃) δ 3.61-3.64 (t, 3H, J=13.2 Hz), 2.40-2.44 (t, 3H, J=14.8 Hz), 2.04 (br s, 2H), δ 1.53-1.63 (m, 8H), 1.31 1.39 (m, 6=4H). ¹³C NMR (100 MHz, CDCl₃) δ 211.7, 62.5, 42.6, 32.3, 25.3, 23.4.

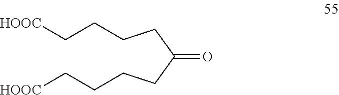 [SMILES: O=C(O)CCCCC(=O)CCCCC(=O)O]

(x) 6-Oxoundecanedioic acid (55). A solution of commercial monoethyl adipate (5.20 g, 29.9 mmol) in SOCl₂ (5.5 mL) was heated to reflux for 2 minutes then cooled to room temperature. Excess SOCl₂ was removed under vacuum. The residue was dissolved in toluene (5 mL) and concentrated to remove any remaining SOCl₂, yielding the crude acid chloride (5.73 g, quantitative), which was used in the next step without purification. ¹H NMR (400 MHz, CDCl₃) δ 4.14 (q, J=7.2 Hz, 2H), 2.92 (t, J=7.0 Hz, 2H), 2.33 (t, J=7.1 Hz, 2H), 1.87-1.60 (m, 4H), 1.26 (t, J=7.2 Hz, 3H). Neat Et₃N (4.15 mL, 29.7 mmol) was added dropwise over the course of 3 minutes to a stirring solution of the above acid chloride (5.73 g, 29.7 mmol) in toluene (50 mL) at 0° C. under an atmosphere of nitrogen. The reaction was warmed to 35° C. and stirred for 15 minutes, then cooled to room temperature and stirred for an additional 30 minutes, at which point a thick white precipitate had formed. The mixture was filtered through a pad of Celite,® and the solid precipitate was washed with more toluene (15 mL). The combined filtrates were concentrated to yield 5-(3-(4-ethoxy-4-oxobutyl)-4-oxooxetan-2-ylidene)pentanoate, which was used directly in the next step without further purification. ¹H NMR (400 MHz, CDCl₃) δ 4.75 (dt, 1H, J₁=7.7, J₂=1.3 Hz), 4.15 (AA′BB′, 4H, app J₁=7.1 Hz), 3.99 (br t, 1H, J=6.9 Hz), 2.40-2.29 (m, 4H), 2.19 (br q, 2H, J=7.5 Hz), 1.89-1.64 (m, 6H), 1.27 (t, 6H, J=7.1 Hz). This compound was suspended in 2 N aq. KOH (25.0 mL) and heated at reflux for 6 hours, whereupon the solution became homogenous. The cooled solution was washed with Et₂O (2×15.0 mL), and the ether extracts were discarded. The solution was then acidified with conc. HCl to pH 2. The aqueous layer was then kept at 0° C. for 1 hour, during which time a precipitate formed. The solid was collected by suction filtration to yield 6-oxo-undecanedioic acid as an off white solid (2.8 g, 62% over two steps). ¹H NMR (400 MHz, CDCl₃) δ 2.44-2.38 (m, 4H), 2.37-2.30 (m, 4H), 1.69-1.55 (m, 8H). MS (negative ion ESI): m/z 229 [M−1]⁻.

### (B) Synthesis of Lipids 5-8

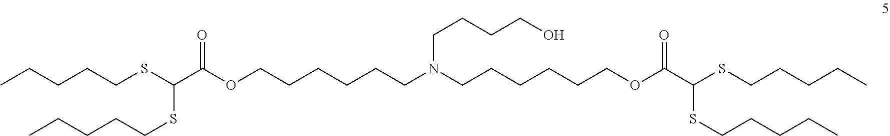 [SMILES: CCCCCSC(SCCCCC)C(=O)OCCCCCCN(CCCCO)CCCCCCOC(=O)C(SCCCCC)SCCCCC]

(i) ((4-Hydroxybutyl)azanediyl)bis(hexane-6,1-diyl) bis(2,2-bis(pentylthio)acetate) (5). A mixture of 36 (150 mg, 0.351 mmol), 4-amino-1-butanol (16 mg, 0.175 mmol) and K₂CO₃ (51 mg, 0.368 mmol) in MeCN (3.5 mL) was stirred at 80° C. in a sealed reaction vessel for 18 hours. The mixture was cooled, diluted with water (5.00 mL) and extracted with CH₂Cl₂ (3×5.00 mL). The combined organics were dried (Na₂SO₄) and concentrated. The residue was purified by silica chromatography (0-5% MeOH in CH₂Cl₂) to yield lipid 5 (85 mg, 62%) as an oil. ¹H NMR (400 MHz, CDCl₃) δ 4.32 (s, 2H), 4.15 (t, J=6.5 Hz, 4H), 3.73 (t, J=5.3 Hz, 2H), 3.10 (t, J=7.9 Hz, 2H), 3.00 (t, J=8.5 Hz, 4H), 2.80-2.58 (m, 8H), 1.95 (t, J=7.8 Hz, 2H), 1.88-1.77 (m, 4H), 1.68 (q, J=6.6 Hz, 6H), 1.59 (p, J=7.3 Hz, 8H), 1.50-1.24 (m, 24H), 0.89 (t, J=7.0 Hz, 12H).

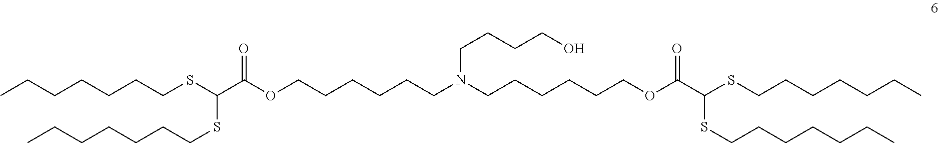 [SMILES: CCCCCCCSC(SCCCCCCC)C(=O)OCCCCCCN(CCCCO)CCCCCCOC(=O)C(SCCCCCCC)SCCCCCCC]

(ii) ((4-Hydroxybutyl)azanediyl)bis(hexane-6,1-diyl) bis(2,2-bis(heptylthio)acetate) (6). Prepared from 37 by the same procedure used for 5. ¹H NMR (400 MHz, CDCl₃) δ 4.32 (s, 2H), 4.14 (t, J=6.7 Hz, 4H), 3.63-3.52 (m, 2H), 2.79-2.59 (m, 8H), 2.58-2.40 (m, 6H), 1.80-1.47 (m, 22H), 1.47-1.18 (m, 38H), 0.97-0.83 (m, 12H).

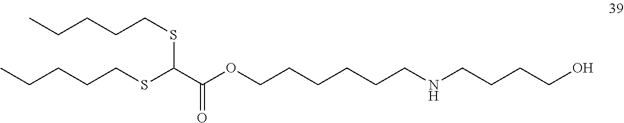 [SMILES: CCCCCSC(SCCCCC)C(=O)OCCCCCCNCCCCO]

(iii) 6-((4-Hydroxybutyl)amino)hexyl 2,2-bis(pentylthio)acetate (39). A solution of 36 (200 mg, 0.468 mmol), 4-amino-1-butanol (43.8 mg, 0.491 mmol) and K₂CO₃ (67.9 mg, 0.491 mmol) in DMF (5.00 mL) was stirred at room temperature for 18 hours under inert atmosphere. The mixture was then diluted with water (10.0 mL) and extracted with Et₂O (3×10.0 mL). The combined organics were washed (brine), dried (Na₂SO₄) and concentrated. The residue was purified by silica chromatography (0-45% MeOH in CH₂Cl₂) to yield 39 (118 mg, 58%). ¹H NMR (400 MHz, CDCl₃) δ 4.30 (s, 1H), 4.13 (t, J=6.7 Hz, 2H), 3.60-3.53 (m, 2H), 2.77-2.54 (m, 8H), 1.74-1.44 (m, 12H), 1.44-1.19 (m, 20H), 0.91-0.82 (m, 6H).

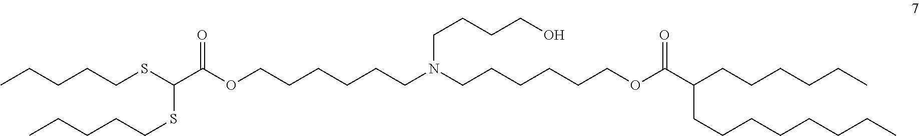 [SMILES: CCCCCCCCC(CCCCCC)C(=O)OCCCCCCN(CCCCO)CCCCCCOC(=O)C(SCCCCC)SCCCCC]

(iv) 6-((6-(2,2-bis(Pentylthio)acetoxy)hexyl)(4-hydroxybutyl)amino)hexyl 2-hexyldecanoate (7). A mixture of 39 (110 mg, 0.252 mmol), 38 (116 mg, 0.278 mmol) and K₂CO₃ (42 mg, 0.302 mmol) in MeCN (2.5 mL) was stirred at 80° C. in a sealed reaction vessel for 18 hours. The mixture was cooled, diluted with water (5.00 mL) and extracted with CH₂Cl₂ (3×5.00 mL). The combined organics were dried (Na₂SO₄) and concentrated. The residue was purified by silica chromatography (0-5% MeOH in CH₂Cl₂) to yield lipid 7 (125 mg, 64%) as an oil. ¹H NMR (400 MHz, CDCl₃) δ 4.32 (s, 1H), 4.15 (t, J=6.6 Hz, 2H), 4.05 (t, J=6.7 Hz, 2H), 3.64-3.54 (m, 2H), 2.79-2.40 (m, 10H), 2.35-2.25 (m, 1H), 1.81-1.49 (m, 18H), 1.48-1.18 (m, 38H), 0.96-0.84 (m, 12H).

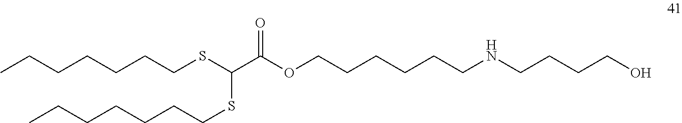 [SMILES: CCCCCCCSC(SCCCCCCC)C(=O)OCCCCCCNCCCCO]

(v) 6-((4-Hydroxybutyl)amino)hexyl 2,2-bis(heptylthio)acetate (41). Prepared from 37 by the same procedure employed for 39. ¹H NMR (400 MHz, CDCl₃) δ 4.30 (s, 1H), 4.13 (t, J=6.7 Hz, 2H), 3.60-3.53 (m, 2H), 2.77-2.54 (m, 8H), 1.74-1.44 (m, 12H), 1.44-1.19 (m, 20H), 0.91-0.82 (m, 6H).

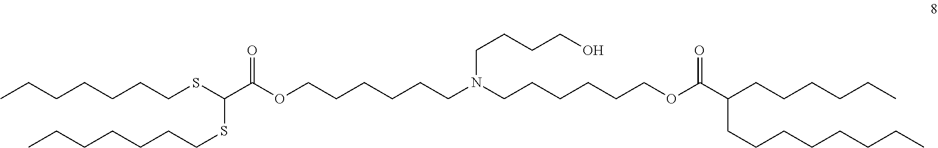 [SMILES: CCCCCCCCC(CCCCCC)C(=O)OCCCCCCN(CCCCO)CCCCCCOC(=O)C(SCCCCCCC)SCCCCCCC]

(vi) 6-((6-(2,2-bis(Heptylthio)acetoxy)hexyl)(4-hydroxybutyl)amino)hexyl 2-hexyldecanoate (8). Prepared from 41 and 38 by the same procedure employed for 7. ¹H NMR (400 MHz, CDCl₃) δ 4.31 (s, 1H), 4.14 (t, J=6.6 Hz, 2H), 4.04 (t, J=6.7 Hz, 2H), 3.67 (t, J=5.4 Hz, 2H), 3.00-2.78 (m, 6H), 2.78-2.57 (m, 4H), 2.38-2.23 (m, 1H), 1.95-1.81 (m, 2H), 1.81-1.51 (m, 16H), 1.50-1.15 (m, 46H), 0.87 (td, J=6.9, 2.5 Hz, 12H).

### (C) Synthesis of Ketone Precursors of Lipids 9-19

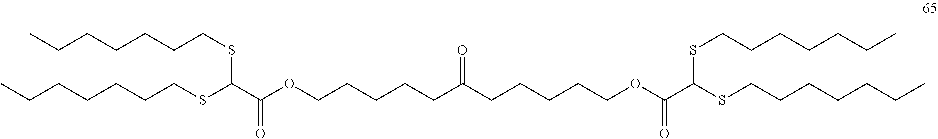 [SMILES: CCCCCCCSC(SCCCCCCC)C(=O)OCCCCCC(=O)CCCCCOC(=O)C(SCCCCCCC)SCCCCCCC]

(i) 6-Oxoundecane-1,11-diyl bis(2,2-bis(heptylthio)acetate) (65). A solution of ketodiol 47 (350 mg, 1.73 mmol), acid 21 (1.22 g, 3.81 mmol), EDCI-HCl (830 mg, 4.33 mmol) and DMAP (529 mg, 4.33 mmol) in CH₂Cl₂ (15.0 mL) was stirred under inert atmosphere for 18 hours then concentrated. The residue was purified by silica chromatography (0-10% EtOAc in Hexanes) to yield ketone 65 (1.20 g, 86%) as an oil. ¹H NMR (400 MHz, CDCl₃) δ 4.31 (s, 2H), 4.15 (t, J=6.7 Hz, 4H), 2.79-2.58 (m, 8H), 2.40 (t, J=7.4 Hz, 4H), 1.75-1.50 (m, 16H), 1.46-1.21 (m, 32H), 0.94-0.83 (m, 12H).

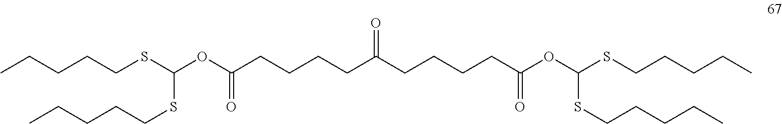 [SMILES: CCCCCSC(OC(=O)CCCCC(=O)CCCCC(=O)OC(SCCCCC)SCCCCC)SCCCCC]

(ii) 6-Oxoundecane-1,11-diyl bis(3,3-bis(pentylthio)propanoate) (67). A solution of ketodiol 47 (203 mg, 1.00 mmol), acid 22 (560 mg, crude), EDCI-HCl (403 mg, 2.10 mmol) and DMAP (256 mg, 2.10 mmol) in CH₂Cl₂ (7.00 mL) was stirred under inert atmosphere for 18 hours then concentrated. The residue was purified by silica chromatography (0-10% EtOAc in Hexanes) to yield ketone 67 (98 mg, 14%) as an oil. ¹H NMR (400 MHz, CDCl₃) δ 4.20 (t, J=7.6 Hz, 2H), 4.10 (t, J=6.7 Hz, 4H), 2.78 (d, J=7.6 Hz, 4H), 2.73-2.52 (m, 8H), 2.40 (t, J=7.4 Hz, 4H), 1.71-1.50 (m, 14H), 1.43-1.21 (m, 22H), 0.94-0.82 (m, 12H).

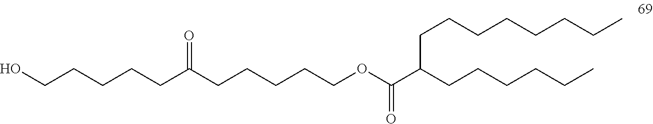 [SMILES: CCCCCCCCC(CCCCCC)C(=O)OCCCCCC(=O)CCCCCO]

(iii) 11-Hydroxy-6-oxoundecyl 2-hexyldecanoate (69). A solution of ketodiol 47 (300 mg, 1.48 mmol), 2-hexyldecanoic acid (362 mg, 1.41 mmol), EDCI-HCl (312 mg, 1.63 mmol) and DMAP (199 mg, 1.63 mmol) were stirred in DCM (6.00 mL) at room temperature under inert atmosphere for 18 hours. The mixture was concentrated, and the residue purified by silica chromatography (0-50%) to yield the monoester 69 as an oil (429 mg, 69%). ¹H NMR (400 MHz, CDCl₃) δ 4.06 (t, J=6.6 Hz, 2H), 3.65 (t, J=6.5 Hz, 2H), 2.41 (td, J=7.4, 3.8 Hz, 4H), 2.36-2.25 (m, 1H), 1.68-1.14 (m, 36H), 0.87 (t, J=6.6 Hz, 6H).

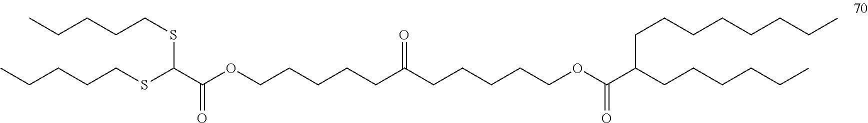 [SMILES: CCCCCCCCC(CCCCCC)C(=O)OCCCCCC(=O)CCCCCOC(=O)C(SCCCCC)SCCCCC]

(iv) 11-(2,2-bis(Pentylthio)acetoxy)-6-oxoundecyl 2-hexyldecanoate (70). A solution of alcohol 69 (800 mg, 1.82 mmol), acid 20 (576 mg, 2.18 mmol), EDCI-HCl (454 mg, 2.37 mmol) and DMAP (290 mg, 2.37 mmol) in CH₂Cl₂ (20.0 mL) was stirred under inert atmosphere at room temperature for 18 hours, then it was concentrated. The residue was purified by silica chromatography (0-10% EtOAc in Hexanes) to yield ketone 70 (1.12 g, 90%) as an oil. ¹H NMR (400 MHz, CDCl₃) δ 4.31 (s, 1H), 4.15 (t, J=6.6 Hz, 2H), 4.06 (t, J=6.6 Hz, 2H), 2.78-2.58 (m, 4H), 2.40 (td, J=7.4, 1.7 Hz, 4H), 2.34-2.24 (m, 1H), 1.85-1.50 (m, 16H), 1.50-1.13 (m, 32H), 0.95-0.80 (m, 12H).

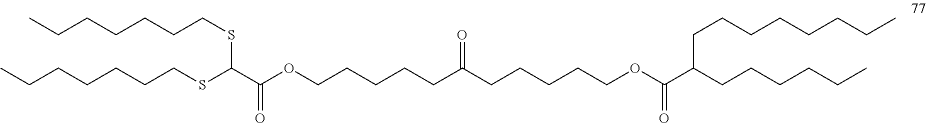 [SMILES: CCCCCCCCC(CCCCCC)C(=O)OCCCCCC(=O)CCCCCOC(=O)C(SCCCCCCC)SCCCCCCC]

(v) 11-(2,2-bis(Heptylthio)acetoxy)-6-oxoundecyl 2-hexyldecanoate (77). Prepared from 69 and 21 by the same procedure employed for 70. ¹H NMR (400 MHz, CDCl₃) δ 4.31 (s 1H), 4.15 (t, J=6.6 Hz, 2H), 4.06 (t, J=6.6 Hz, 2H), 2.78-2.58 (m, 4H), 2.40 (td, J=7.4, 1.7 Hz, 4H), 2.34-2.24 (m, 1H), 1.85-1.50 (m, 20H), 1.50-1.13 (m, 36H), 0.95-0.80 (m, 12H).

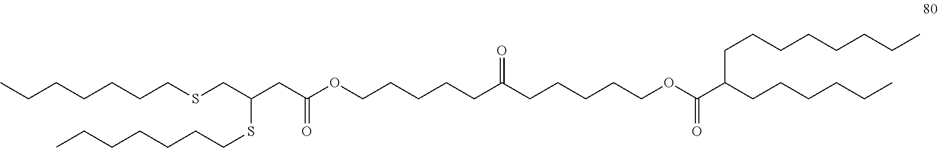 [SMILES: CCCCCCCCC(CCCCCC)C(=O)OCCCCCC(=O)CCCCCOC(=O)CC(CSCCCCCCC)SCCCCCCC]

(vi) 11-((3,4-bis(Heptylthio)butanoyl)oxy)-6-oxoundecyl 2-hexyldecanoate (80). Prepared from 69 and 23 by the same procedure employed for 70. ¹H NMR (400 MHz, CDCl₃) δ 4.13-4.02 (m, 4H), 3.23-3.10 (m, 1H), 3.00-2.85 (m, 2H), 2.75-2.45 (m, 6H), 2.40 (t, J=7.4 Hz, 4H), 2.34-2.23 (m, 1H), 1.74-1.50 (m, 16H), 1.48-1.18 (m, 40H), 0.94-0.82 (m, 12H).

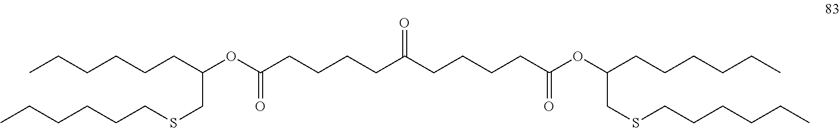 [SMILES: CCCCCCSCC(CCCCCC)OC(=O)CCCCC(=O)CCCCC(=O)OC(CCCCCC)CSCCCCCC]

(vii) bis(1-(Hexylthio)octan-2-yl) 6-oxoundecanedioate (83). A solution of ketodiacid 55 (1.0 g, 4.34 mmol), alcohol 24 (2.35 g, 9.55 mmol), EDCI-HCl (2.1 g, 10.4 mmol) and DMAP (0.53 g, 4.34 mmol) in CH₂Cl₂ (15.0 mL) was stirred under inert atmosphere for 18 hours. The solution was diluted with more CH₂Cl₂ (15.0 mL) and sequentially washed with aqueous saturated NaHCO₃ (2×15.0 mL) and water (15.0 mL), then dried (Na₂SO₄) and evaporated. The residue was purified by silica chromatography (10% EtOAc in hexanes) to yield desired product (2.0 g, 67%). ¹H NMR (400 MHz, CDCl₃) δ 5.00-4.86 (m, 2H), 2.63 (d, J=6.13 Hz, 4H), 2.52 (t, J=7.39 Hz, 4H), 2.47-2.36 (m, 4H), 2.37-2.25 (m, 4H), 1.78-1.47 (m, 14H), 1.45-1.18 (m, 30H), 0.87 (t, J=6.89, 12H). LRMS m/z 709 [M+Na]⁺.

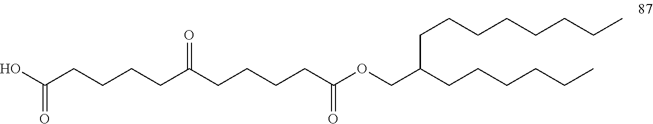 [SMILES: CCCCCCCCC(CCCCCC)COC(=O)CCCCC(=O)CCCCC(=O)O]

(viii) 11-((2-Hexyldecyl)oxy 6,11-dioxoundecanoic acid (87). Obtained from ketodiacid 55 and 1 mol equivalent of 2-hexyl-1-decanol by procedure (vii) above by using 1.5 mol equiv of EDCI. ¹H NMR (400 MHz, CDCl₃) δ 3.95 (d, J=5.80 Hz, 2H), 2.46-2.25 (m, 8H), 1.66-1.51 (m, 9H), 1.34-1.19 (m, 24H), 0.86 (t, J=6.70 Hz, 6H). LRMS: 455 [M+H]⁺.

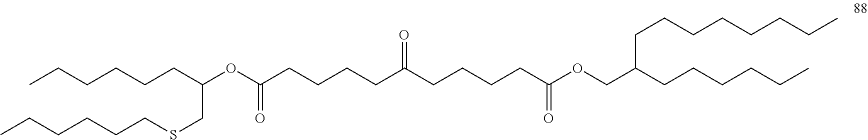 [SMILES: CCCCCCCCC(CCCCCC)COC(=O)CCCCC(=O)CCCCC(=O)OC(CCCCCC)CSCCCCCC]

(ix) 1-(2-Hexyldecyl) 11-(1-(hexylthio)octan-2-yl) 6-oxoundecanedioate (88). A solution of 87 (1.0 g, 2.2 mmol), 1-hexylsulfanyloctan-2-ol, 24, (0.65 g, 2.64 mmol), DMAP (0.27 g, 2.2 mmol), and EDCI*HCl (0.63 g, 3.3 mmol) in DCM (8.0 mL), was stirred under inert atmosphere at room temperature for 18 hours. The resulting mixture was diluted with DCM (10.0 mL), sequentially washed with sat. aq. NaHCO₃ solution (2×10.0 mL), water (2×10.0 mL), dried (Na₂SO₄) and concentrated under reduced pressure. This residue was purified by silica gel column chromatography with 4% EtOAc/hexanes to provide the desired product (1.0 g, 66% yield) as a colorless oil. ¹H NMR (400 MHz, CDCl₃) δ 5.00-4.85 (m, 1H), 3.95 (d, J=5.82 Hz, 2H), 2.63 (d, J=6.13 Hz, 2H), 2.58-2.47 (m, 2H), 2.46-2.35 (m, 4H), 2.34-2.24 (m, 4H), 1.77-1.45 (m, 15H), 1.44-1.16 (m, 36H), 0.87 (m, 12H). LRMS: 705 [M+Na]⁺.

### (D) Synthesis of Lipids 9-17

(i) General procedure for selective reduction of the ketone. Solid NaBH₄ (38 mg, 1 molar equivalent) was added portionwise to a cold (0° C.) solution of ketone (1 mmol) in EtOH (10 mL). The mixture was warmed to room temperature and stirred for 30 minutes. The reaction was carefully quenched with sat. aq. NH₄Cl (3 mL), diluted with water (5 mL) and extracted with CH₂Cl₂ (3×5.00 mL). The combined organics were dried (Na₂SO₄) and concentrated to yield the alcohol as an oil and in essentially quantitative yield. The alcohol was used in the next step without further purification. The following alcohols were thus prepared.

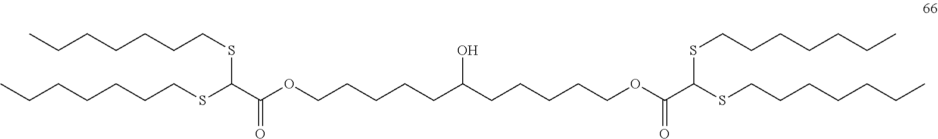 [SMILES: CCCCCCCSC(SCCCCCCC)C(=O)OCCCCCC(O)CCCCCOC(=O)C(SCCCCCCC)SCCCCCCC]

(ii) 6-Hydroxyundecane-1,11-diyl bis(2,2-bis(heptylthio)acetate) (66). Obtained from ketone 65. ¹H NMR (400 MHz, CDCl₃) δ 4.32 (s, 2H), 4.16 (t, J=6.7 Hz, 4H), 3.62-3.54 (m, 1H), 2.81-2.57 (m, 8H), 1.78-1.15 (m, 56H), 0.96-0.83 (m, 12H).

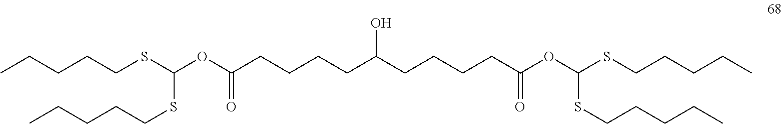 [SMILES: CCCCCSC(OC(=O)CCCCC(O)CCCCC(=O)OC(SCCCCC)SCCCCC)SCCCCC]

(iii) 6-Hydroxyundecane-1,11-diyl bis(3,3-bis(pentylthio)propanoate) (68). Obtained from ketone 67. ¹H NMR (400 MHz, CDCl₃) δ 4.20 (t, J=7.6 Hz, 2H), 4.12 (t, J=6.7 Hz, 4H), 3.61-3.54 (m, 1H), 2.78 (d, J=7.6 Hz, 4H), 2.73-2.50 (m, 8H), 1.71-1.51 (m, 12H), 1.49-1.23 (m, 28H), 0.89 (t, J=7.0 Hz, 12H).

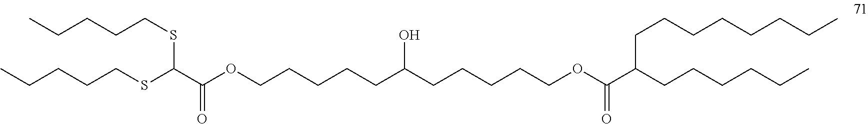 [SMILES: CCCCCCCCC(CCCCCC)C(=O)OCCCCCC(O)CCCCCOC(=O)C(SCCCCC)SCCCCC]

(iv) 11-(2,2-bis(pentylthio)acetoxy)-6-hydroxyundecyl 2-hexyldecanoate (71). Obtained from ketone 70. ¹H NMR (400 MHz, CDCl₃) δ 4.32 (s, 1H), 4.16 (t, J=6.7 Hz, 2H), 4.07 (t, J=6.6 Hz, 2H), 3.61-3.54 (m, 1H), 2.83-2.54 (m, 4H), 2.37-2.23 (m, 1H), 1.75-1.17 (m, 52H), 0.99-0.79 (m, 12H).

(v) General procedure for the preparation of ω-(dimethylamino)alkanoate esters. A solution of an alcohol (0.5 mmol), an ω-(dimethylamino)alkanoic acid hydrochloride (1.2 equiv, 0.6 mmol), EDCI-HCl (1.4 equiv, 0.7 mmol) and DMAP (1.4 equiv, 7 mmol) in CH₂Cl₂ (5 mL) was stirred under inert atmosphere at room temperature for 18 hours then concentrated. The residue was purified by silica chromatography (0-5% MeOH in CH₂Cl₂) to yield the ω-(dimethylamino)alkanoate ester of the starting alcohol (85-95% for a primary alcohol, 70-80% for a secondary alcohol) as an oil. The following lipids were thus obtained:

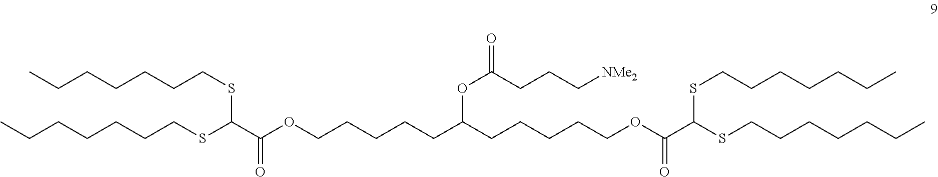 [SMILES: CCCCCCCSC(SCCCCCCC)C(=O)OCCCCCC(CCCCCOC(=O)C(SCCCCCCC)SCCCCCCC)OC(=O)CCCN(C)C]

(vi) 7-((4-(Dimethylamino)butanoyl)oxy)tridecane-1,13-diyl bis(2,2-bis(heptylthio)acetate) (9). Obtained from alcohol 66 and 4-(dimethylamino)butanoic acid hydrochloride. ¹H NMR (400 MHz, CDCl₃) δ 4.94-4.79 (m, 1H), 4.30 (s, 1H), 4.12 (t, J=6.7 Hz, 4H), 2.76-2.57 (m, 8H), 2.36-2.24 (m, 4H), 2.20 (s, 6H), 1.77 (p, J=7.5 Hz, 2H), 1.69-1.07 (m, 61H), 0.92-0.78 (m, 12H).

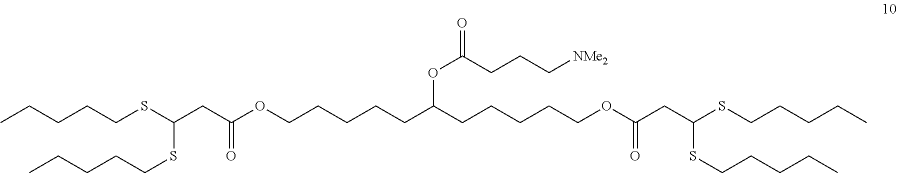 [SMILES: CCCCCSC(CC(=O)OCCCCCC(CCCCCOC(=O)CC(SCCCCC)SCCCCC)OC(=O)CCCN(C)C)SCCCCC]

(vii) 6-((4-(dimethylamino)butanoyl)oxy)undecane-1,11-diyl bis(3,3-bis(pentylthio) prop-anoate) (10). Obtained from alcohol 68 and 4-(dimethylamino)butanoic acid hydrochloride. ¹H NMR (400 MHz, CDCl₃) δ 4.85 (p, J=6.0 Hz, 1H), 4.19 (t, J=7.6 Hz, 2H), 4.09 (t, J=6.7 Hz, 4H), 2.77 (d, J=7.6 Hz, 4H), 2.72-2.52 (m, 8H), 2.42 (t, J=7.5 Hz, 2H), 2.38-2.28 (m, 8H), 1.85 (p, J=7.4 Hz, 2H), 1.71-1.45 (m, 16H), 1.43-1.21 (m, 24H), 0.88 (t, J=7.0 Hz, 12H).

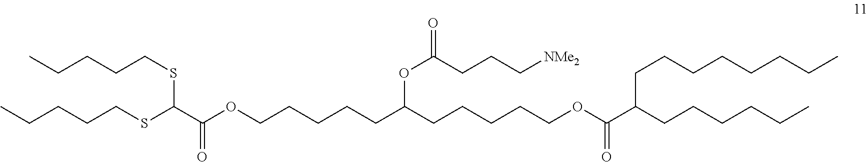 [SMILES: CCCCCCCCC(CCCCCC)C(=O)OCCCCCC(CCCCCOC(=O)C(SCCCCC)SCCCCC)OC(=O)CCCN(C)C]

(viii) 11-(2,2-bis(Pentylthio)acetoxy)-6-((4-(dimethylamino)butanoyl)oxy)undecyl 2-hexyl-decanoate (11). Obtained from alcohol 71 and 4-(dimethylamino)butanoic acid hydrochloride. ¹H NMR (400 MHz, CDCl₃) δ 4.94-4.79 (m, 1H), 4.30 (s, 1H), 4.12 (t, J=6.7 Hz, 2H), 4.02 (t, J=6.7 Hz, 2H), 2.76-2.57 (m, 4H), 2.36-2.24 (m, 4H), 2.20 (s, 6H), 1.77 (p, J=7.5 Hz, 2H), 1.69-1.07 (m, 53H), 0.92-0.78 (m, 12H).

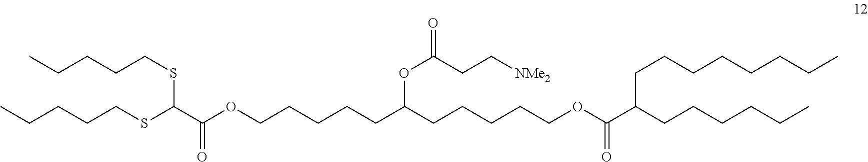 [SMILES: CCCCCCCCC(CCCCCC)C(=O)OCCCCCC(CCCCCOC(=O)C(SCCCCC)SCCCCC)OC(=O)CCN(C)C]

(ix) 11-(2,2-bis(pentylthio)acetoxy)-6-((3-(dimethylamino)propanoyl)oxy)undecyl 2-hexyldecanoate (12). Obtained from alcohol 71 and 3-(dimethylamino)propanoic acid hydrochloride. ¹H NMR (400 MHz, CDCl₃) δ 4.87 (p, J=6.1 Hz, 1H), 4.31 (s, 1H), 4.13 (t, J=6.7 Hz, 2H), 4.04 (t, J=6.7 Hz, 2H), 2.76-2.56 (m, 6H), 2.45 (t, J=7.3 Hz, 2H), 2.34-2.25 (m, 1H), 2.23 (s, 6H), 1.80-1.11 (m, 52H), 1.02-0.76 (m, 12H).

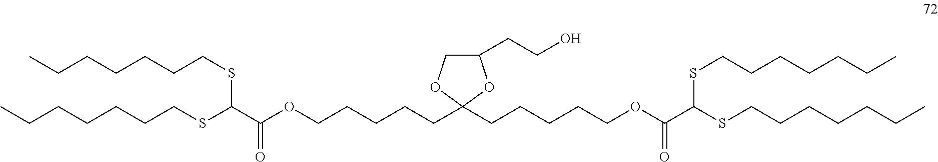 [SMILES: CCCCCCCSC(SCCCCCCC)C(=O)OCCCCCC1(CCCCCOC(=O)C(SCCCCCCC)SCCCCCCC)OCC(CCO)O1]

(x) Exemplary procedure for hydroxyketal formation: (4-(2-hydroxyethyl)-1,3-dioxolane-2,2-diyl)bis(pentane-5,1-diyl) bis(2,2-bis(heptylthio)acetate) (72). A solution of ketone 65 (700 mg, 0.868 mmol), 1,2,4-butanetriol (184.2 mg, 1.74 mmol) and pyridinium p-toluenesulfonate (43.6 mg, 0.174 mmol) in toluene (15.0 mL) was refluxed under nitrogen overnight with continuous removal of water (Dean-Stark trap). The mixture was cooled to room temperature, washed with water (2×10.0 mL), brine (10.0 mL) then dried (Na₂SO₄) and concentrated. The residue was purified by silica gel column chromatography (0-5% MeOH in CH₂Cl₂) to yield ketal 72 (396 mg, 51%) as an oil. 1H NMR (400 MHz, CDCl₃) δ 4.32 (s, 2H), 4.28-4.19 (m, 1H), 4.15 (td, J=6.7, 1.3 Hz, 4H), 4.09 (dd, J=7.9, 6.0 Hz, 1H), 3.80 (td, J=6.0, 2.5 Hz, 2H), 3.52 (t, J=8.1 Hz, 1H), 2.80-2.58 (m, 8H), 1.81 (q, J=6.0 Hz, 2H), 1.73-1.49 (m, 18H), 1.46-1.21 (m, 38H), 0.93-0.83 (m, 12H).

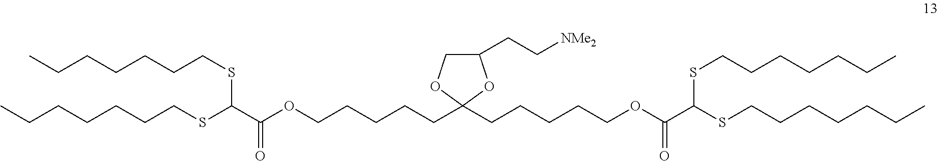 [SMILES: CCCCCCCSC(SCCCCCCC)C(=O)OCCCCCC1(CCCCCOC(=O)C(SCCCCCCC)SCCCCCCC)OCC(CCN(C)C)O1]

(xi) Exemplary procedure for hydroxyketal tosylation and tosylate displacement: (4-(2-(dimethylamino)ethyl)-1,3-dioxolane-2,2-diyl)bis(pentane-5,1-diyl) bis(2,2-bis(heptylthio)-acetate) (13). To a solution of ketal 72 (198 mg, 0.221 mmol), TEA (0.0462 mL, 0.332 mmol) and DMAP (2.70 mg, 0.0221 mmol) in CH₂Cl₂ (2.00 mL) was added TsCl (50.6 mg, 0.265 mmol) at 0° C. under inert atmosphere. The reaction was warmed to room temperature and stirred for 18 hours. The reaction was quenched with water (3.00 mL) and extracted with CH₂Cl₂ (3×4.00 mL). The combined organics were dried (Na₂SO₄) and concentrated to yield tosylate 73 (248 mg, crude) which was used in the next step without further purification. ¹H NMR (400 MHz, CDCl₃) δ 7.79 (d, J=8.3 Hz, 2H), 7.36 (d, J=8.0 Hz, 2H), 4.32 (s, 2H), 4.21-4.07 (m, 7H), 4.06-3.99 (m, 1H), 3.49-3.41 (m, 1H), 2.79-2.58 (m, 8H), 2.46 (s, 3H), 1.90 (q, J=6.4 Hz, 2H), 1.72-1.46 (m, 18H), 1.43-1.19 (m, 38H), 0.93-0.83 (m, 12H). The above tosylate (248 mg, crude), dimethyl amine (3.00 mL, 2 M in THF) and MeOH (3.00 mL) was heated in a microwave reactor (110° C., normal absorbance) for 15 minutes. The mixture was then concentrated, and the residue purified by silica chromatography (0-5% MeOH in CH₂Cl₂) to yield lipid 13 (192 mg, 94% over 2 steps) as an oil. ¹H NMR (400 MHz, CDCl₃) δ 4.32 (s, 2H), 4.19-4.12 (m, 4H), 4.11-4.03 (m, 2H), 3.52-3.41 (m, 1H), 2.79-2.59 (m, 8H), 2.56-2.23 (m, 8H), 1.91-1.50 (m, 20H), 1.45-1.22 (m, 38H), 0.92-0.84 (m, 12H).

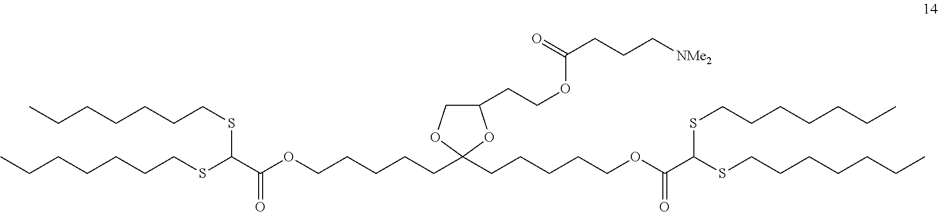 [SMILES: CCCCCCCSC(SCCCCCCC)C(=O)OCCCCCC1(CCCCCOC(=O)C(SCCCCCCC)SCCCCCCC)OCC(CCOC(=O)CCCN(C)C)O1]

(xii) Exemplary procedure for the preparation of a hydroxyketal)-(dimethyl-amino)alkanoate ester: (4-(2-((4-(dimethylamino)butanoyl)oxy)ethyl)-1,3-dioxolane-2,2-diyl)bis(pentane-5,1-diyl) bis(2,2-bis(heptylthio)acetate) (14). Prepared from hydroxyketal 72 and 4-(dimethylamino)butanoic acid hydrochloride according to general procedure (v) above. ¹H NMR (400 MHz, CDCl₃) δ 4.32 (s, 2H), 4.27-4.19 (m, 1H), 4.19-4.04 (m, 7H), 3.49 (t, J=7.7 Hz, 1H), 2.82-2.58 (m, 8H), 2.40-2.29 (m, 4H), 2.25 (s, 6H), 2.04-1.75 (m, 4H), 1.73-1.50 (m, 16H), 1.44-1.19 (m, 40H), 0.95-0.83 (m, 12H).

(xiii) General procedure for the reductive amination of a ketone. To a solution of ketone (1.0 mmol) and an O-protected derivative of an aminoalcohol (2.0 mmol) in 1,2-dichloroethane (5.00 mL) was added NaBH(OAc)₃ (1.8 mmol) and HOAc (0.1 mL). The resulting mixture was stirred under inert atmosphere at room temperature for 18 hours then quenched with sat. aq. NaHCO₃ (3 mL), diluted with water (4 mL) and extracted with CH₂Cl₂ (3×10 mL). The combined organics were dried (Na₂SO₄) and concentrated. The residue was purified by silica chromatography (0-5% MeOH in CH₂Cl₂) to yield the secondary amine (70-80% yield) as an oil. The following secondary amines were thus prepared.

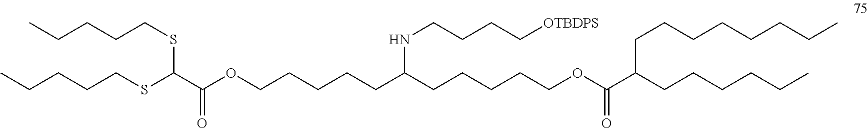

(xiv) 11-(2,2-bis(Pentylthio)acetoxy)-6-((4-((tert-butyldiphenylsilyl)oxy)butyl)amino)-undecyl 2-hexyldecanoate (75). Obtained from ketone 70 and 4-((tert-butyldiphenylsilyl)oxy)-butan-1-amine, 74. ¹H NMR (400 MHz, CDCl₃) δ 7.75-7.62 (m, 4H), 7.49-7.30 (m, 6H), 4.32 (s, 1H), 4.15 (t, J=6.7 Hz, 2H), 4.05 (t, J=6.6 Hz, 2H), 3.77-3.72 (m, 2H), 3.70-3.63 (m, 2H), 2.79-2.59 (m, 4H), 2.59-2.39 (m, 3H), 2.34-2.24 (m, 1H), 1.73-1.48 (m, 16H), 1.46-1.18 (m, 40H), 1.04 (s, 9H), 0.94-0.83 (m, 12H).

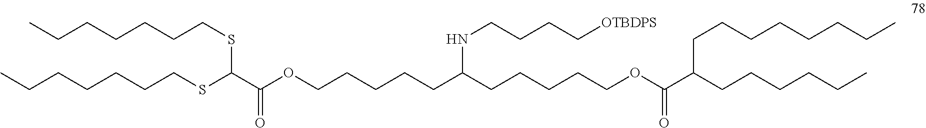

(xv) 11-(2,2-bis(Heptylthio)acetoxy)-6-((4-((tert-butyldiphenylsilyl)oxy)butyl)amino)undec-yl 2-hexyldecanoate (78). Obtained from ketone 77 and 4-((tert-butyldiphenylsilyl)oxy)-butan-1-amine, 74. H NMR (400 MHz, CDCl₃) δ 7.75-7.62 (m, 4H), 7.49-7.30 (m, 6H), 4.32 (s, 1H), 4.15 (t, J=6.7 Hz, 2H), 4.05 (t, J=6.6 Hz, 2H), 3.77-3.72 (m, 2H), 3.70-3.63 (m, 2H), 2.79-2.59 (m, 4H), 2.59-2.39 (m, 3H), 2.34-2.24 (m, 1H), 1.73-1.48 (m, 20H), 1.46-1.18 (m, 44H), 1.04 (s, 9H), 0.94-0.83 (m, 12H).

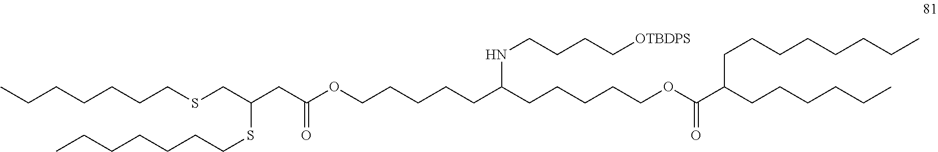

(xvi) 11-((3,4-bis(Heptylthio)butanoyl)oxy)-6-((4-((tert-butyldiphenylsilyl)oxy)butyl)amino) undecyl 2-hexyldecanoate (81). Obtained from ketone 80 and 4-((tert-butyldiphenylsilyl)oxy)-butan-1-amine, 74. ¹H NMR (400 MHz, CDCl₃) δ 7.72-7.61 (m, 4H), 7.50-7.32 (m, 6H), 4.14-4.00 (m, 4H), 3.71-3.63 (m, 2H), 3.18 (tt, J=8.6, 5.2 Hz, 1H), 2.92 (ddd, J=12.2, 8.2, 5.2 Hz, 2H), 2.73-2.43 (m, 9H), 2.30 (tt, J=8.9, 5.2 Hz, 1H), 1.59 (dp, J=20.8, 6.2 Hz, 16H), 1.48-1.19 (m, 48H), 1.04 (s, 9H), 0.92-0.82 (m, 12H).

(xvii) General procedure for reductive methylation of a secondary amine. A mixture of an above secondary amine (1.0 mmol), aq. formaldehyde (37%, 10 mL) and NaBH(OAc)₃ (5.0 mmol) in THF (20 mL) was stirred under inert atmosphere at room temperature for 3 days. The reaction was then quenched with sat. aq. NaHCO₃ (8 mL), diluted with water (15.00 mL) and extracted with CH₂Cl₂ (3×20 mL). The combined organics were dried (Na₂SO₄) and concentrated. The residue was purified by silica chromatography (0-5% MeOH in CH₂Cl₂) to yield the tertiary amine (85-95% yield) as an oil. The following tertiary amines were thus obtained:

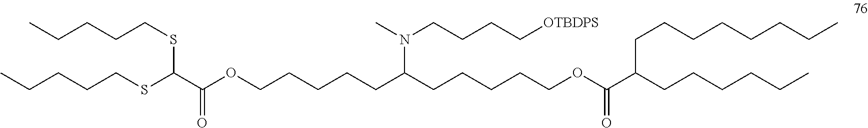

(xviii) 11-(2,2-bis(Pentylthio)acetoxy)-6-((4-((tert-butyldiphenylsilyl)oxy)butyl)(methyl)-amino) undecyl 2-hexyldecanoate (76). Obtained from secondary amine 75. ¹H NMR (400 MHz, CDCl₃) δ 7.77-7.64 (m, 4H), 7.48-7.32 (m, 6H), 4.32 (s, 1H), 4.14 (t, J=6.7 Hz, 2H), 4.05 (t, J=6.7 Hz, 2H), 3.66 (t, J=6.2 Hz, 2H), 2.78-2.58 (m, 4H), 2.38-2.26 (m, 4H), 2.12 (s, 3H), 1.76-1.15 (m, 56H), 1.04 (s, 9H), 0.95-0.83 (m, 12H).

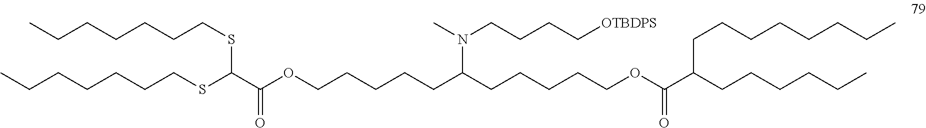

(xix) 11-(2,2-bis(heptylthio)acetoxy)-6-((4-((tert-butyldiphenylsilyl)oxy)butyl)(methyl)-amino) undecyl 2-hexyldecanoate (79). Obtained from secondary amine 78. ¹H NMR (400 MHz, CDCl₃) δ 7.77-7.64 (m, 4H), 7.48-7.32 (m, 6H) 4.32 (s, 1H), 4.14 (t, J=6.7 Hz, 2H), 4.05 (t, J=6.7 Hz, 2H), 3.66 (t, J=6.2 Hz, 2H), 2.78-2.58 (m, 4H), 2.38-2.26 (m, 4H), 2.12 (s, 3H), 1.76-1.15 (m, 64H), 1.04 (s, 9H), 0.95-0.83 (m, 12H).

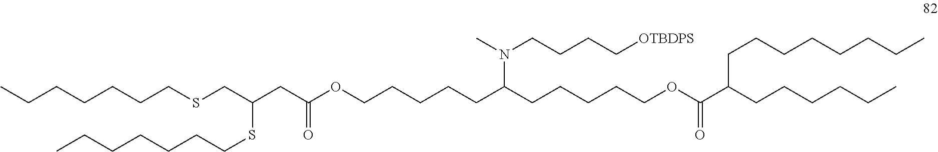

(xx) 11-((3,4-bis(Heptylthio)butanoyl)oxy)-6-((4-((tert-butyldiphenylsilyl)oxy)butyl)-(methyl)amino)undecyl 2-hexyldecanoate (82). Obtained from secondary amine 81. ¹H NMR (400 MHz, CDCl₃) δ 7.73-7.60 (m, 4H), 7.51-7.32 (m, 6H), 4.15-4.00 (m, 4H), 3.66 (t, J=6.2 Hz, 2H), 3.18 (tt, J=9.1, 5.2 Hz, 1H), 2.92 (ddd, J=11.8, 7.7, 5.2 Hz, 2H), 2.71-2.45 (m, 6H), 2.39-2.25 (m, 4H), 2.13 (s, 3H), 1.74-1.13 (m, 64H), 1.04 (s, 9H), 0.93-0.80 (m, 12H).

(xxi) General procedure for desilylation. To a solution of an above silyl ether (0.3 mmol) in THF (3 mL) was added HF-pyridine (0.4 mL) at 0° C. under inert atmosphere. The reaction was warmed to room temperature and stirred for 18 hours. Water (7 mL) was added, and the mixture was extracted with CH₂Cl₂ (3×7.00 mL). The combined organics were dried (Na₂SO₄) and concentrated. The residue was purified by silica chromatography (0-5% MeOH in CH₂Cl₂) to yield a with a type 7 ionizable head group (70-75%) as an oil. The following lipids were thus obtained:

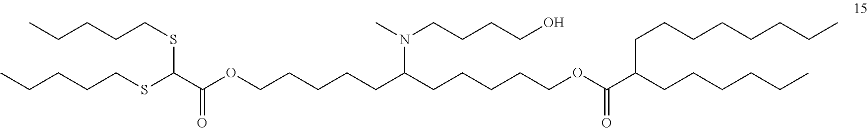 [SMILES: CCCCCCCCC(CCCCCC)C(=O)OCCCCCC(CCCCCOC(=O)C(SCCCCC)SCCCCC)N(C)CCCCO]

(xxii) 11-(2,2-bis(Pentylthio)acetoxy)-6-((4-hydroxybutyl)(methyl)amino)undecyl 2-hexyl-decanoate (15). Obtained from silyl ether 76. ¹H NMR (400 MHz, CDCl₃) δ 4.32 (s, 1H), 4.15 (t, 6.7 Hz, 2H), 4.05 (t, J=6.7 Hz, 2H), 3.60-3.52 (m, 2H), 2.80-2.58 (m, 4H), 2.51-2.39 (m, 3H), 2.37-2.24 (m, 1H), 2.15 (s, 3H), 1.73-1.13 (m, 56H), 0.97-0.80 (m, 12H).

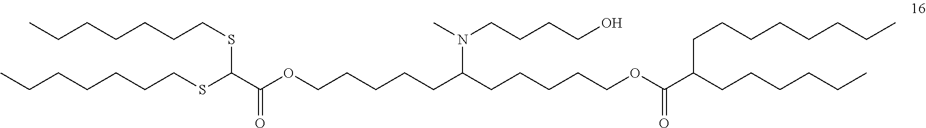 [SMILES: CCCCCCCCC(CCCCCC)C(=O)OCCCCCC(CCCCCOC(=O)C(SCCCCCCC)SCCCCCCC)N(C)CCCCO]

(xxiii) 11-(2,2-bis(Heptylthio)acetoxy)-6-((4-hydroxybutyl)(methyl)amino)undecyl 2-hexyldecanoate (16). Obtained from silyl ether 79. ¹H NMR (400 MHz, CDCl₃) δ 4.32 (s, 1H), 4.15 (t, J=6.7 Hz, 2H), 4.05 (t, J=6.7 Hz, 2H), 3.60-3.52 (m, 2H), 2.80-2.58 (m, 4H), 2.51-2.39 (m, 3H), 2.37-2.24 (m, 1H), 2.15 (s, 3H), 1.73-1.13 (m, 64H), 0.97-0.80 (m, 12H).

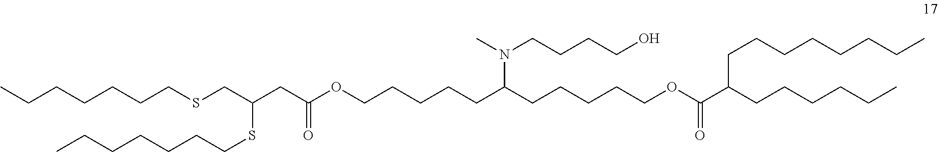 [SMILES: CCCCCCCCC(CCCCCC)C(=O)OCCCCCC(CCCCCOC(=O)CC(CSCCCCCCC)SCCCCCCC)N(C)CCCCO]

(xxiv) 11-((3,4-bis(Heptylthio)butanoyl)oxy)-6-((4-hydroxybutyl)(methyl)amino)undecyl 2-hexyldecanoate (17). Obtained from silyl ether 82. This compound was characterized as the hydrochloride salt. H NMR (400 MHz, CDCl₃) δ 4.17-4.01 (m, 4H), 3.73 (t, J=5.3 Hz, 2H), 3.21-3.12 (m, 2H), 3.09 (t, J=6.5 Hz, 2H), 2.97-2.88 (m, 2H), 2.77 (s, 3H), 2.65 (dd, J=13.6, 9.3 Hz, 1H), 2.59-2.46 (m, 5H), 2.36-2.25 (m, 1H), 1.93 (q, J=6.4 Hz, 2H), 1.84-1.14 (m, 62H), 0.87 (td, J=6.8, 2.6 Hz, 12H).

### (E) Synthesis of Lipids 18-19.

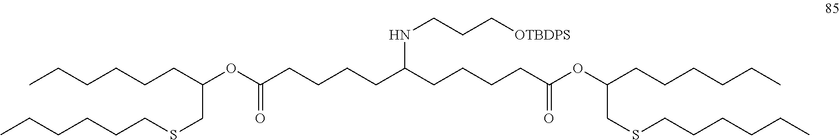

(i) bis(1-(Hexylthio)octan-2-yl) 6-((3-((tert-butyldiphenylsilyl)oxy)propyl)amino)-undecanedioate (85). To a solution of ketone 83 (1.0 g, 1.45 mmol) and OTBDPS-protected amino propanol 84 (0.68 g 0.2 mmol) in DCE (5.0 mL) was added NaBH(OAc)₃ (0.61 g, 2.9 mmol) and HOAc (0.14 mmol). The resulting mixture was stirred under inert atmosphere at room temperature for 18 hours then quenched with sat. aq. NaHCO₃ (5.0 mL), diluted with water (5.0 mL) and extracted with DCM (3×10.0 mL). The combined organics were dried (Na₂SO₄) and concentrated. The residue was purified by silica chromatography (5% MeOH in DCM) to yield secondary amine 85 (1.0 g, 70% yield) as an oil. 1H NMR (400 MHz, CDCl₃) δ 7.83-7.58 (m, 4H), 7.50-7.33 (m, 6H), 4.98-4.91 (m, 2H), 3.73 (t, J=5.93 Hz, 2H), 2.78-2.65 (m, 2H), 2.63 (d, J=6.16 Hz, 4H), 2.58-2.44 (m, 5H), 2.29 (t, J=7.62 Hz, 4H), 1.80-1.49 (m, 10H), 1.46-1.17 (m, 40H), 1.04 (s, 9H), 0.87 (t, J=6.89, 12H). LRMS m/z 984 [M+H]⁺.

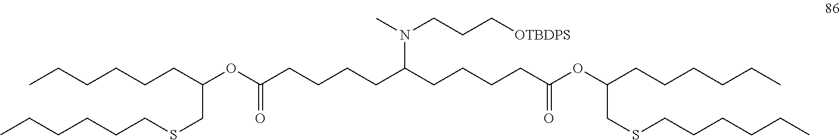

(ii) bis(1-(hexylthio)octan-2-yl) 6-((3-((tert-butyldiphenylsilyl)oxy)propyl)(methyl)amino)-undecanedioate (86). A solution of 85 (0.9 g, 0.9 mmol), sodium triacetoxyborohydride (0.39 g, 1.8 mmol), aq. formaldehyde (37%, 0.27 mL), and HOAc (0.09 mmol) was stirred in THF (2.0 mL) under nitrogen for 18 hours. The reaction was quenched with sat. aq. NaHCO₃ (2.0 mL), diluted with water (5.0 mL) and extracted with CH₂Cl₂ (3×5.0 mL). The combined extracts were dried (Na₂SO₄) and concentrated. The residue was purified by silica chromatography (4% MeOH in DCM) to yield tertiary amine 86 (0.77 g, 85%). ¹H NMR (400 MHz, CDCl₃) δ 7.87-7.55 (m, 4H), 7.50-7.33 (m, 6H), 5.02-4.81 (m, 2H), 3.69 (t, J=6.30 Hz, 2H), 2.75-2.58 (m, 4H), 2.56-2.47 (m, 4H), 2.44 (t, J=7.10 Hz, 2H), 2.34-2.20 (m, 5H), 2.11 (s, 3H), 1.82-1.49 (m, 12H), 1.46-1.09 (m, 38H), 1.04 (s, 9H), 0.93-0.82 (m, 12H). LRMS m/z 998 [M+H]⁺.

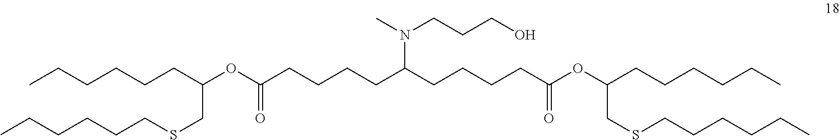 [SMILES: CCCCCCSCC(CCCCCC)OC(=O)CCCCC(CCCCC(=O)OC(CCCCCC)CSCCCCCC)N(C)CCCO]

(iii) bis(1-(Hexylthio)octan-2-yl) 6-((3-hydroxypropyl)(methyl)amino)undecanedioate (18). To a solution of 86 (0.50 g, 0.5 mmol.) in THF (1.0 mL) was added HF-pyridine (0.30 mL, 1.0 mmol) at 0° C. under inert atmosphere. The reaction was warmed to room temperature and stirred for 4 hours. Water (5.0 mL) was added, and the mixture was extracted with CH₂Cl₂ (3×5.0 mL). The combined organics were dried (Na₂SO₄) and concentrated. The residue was purified by silica chromatography (3% MeOH in DCM) to yield product (0.21 g, 53%). ¹H NMR (400 MHz, CDCl₃) δ 5.37 (br, 1H), 5.02-4.87 (m, 2H), 3.78 (t, J=5.17 Hz, 2H), 2.77-2.60 (m, 6H), 2.57-2.41 (m, 5H), 2.31 (t, J=7.58 Hz, 4H), 2.19 (s, 3H), 1.78-1.14 (m, 50H), 0.93-0.80 (m, 12H). LRMS m/z 760 [M+H]+.

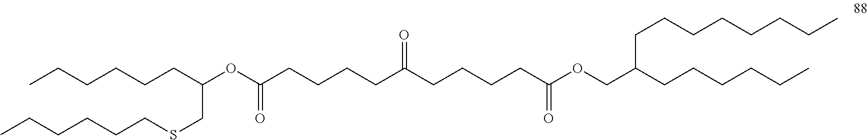 [SMILES: CCCCCCCCC(CCCCCC)COC(=O)CCCCC(=O)CCCCC(=O)OC(CCCCCC)CSCCCCCC]

(iv) 1-(2-Hexyldecyl) 11-(1-(hexylthio)octan-2-yl) 6-oxoundecanedioate (88). A solution of 87 (1.0 g, 2.2 mmol), 1-hexylsulfanyloctan-2-ol, 24, (0.65 g, 2.64 mmol), DMAP (0.27 g, 2.2 mmol), and EDCI*HCl (0.63 g, 3.3 mmol) in CH₂Cl₂ (8.0 mL), was stirred under inert atmosphere at room temperature for 18 hours. The resulting mixture was diluted with CH₂Cl₂ (10.0 mL), sequentially washed with sat. aq. NaHCO₃ solution (2×10.0 mL), water (2×10.0 mL), dried (Na₂SO₄) and concentrated under reduced pressure. This residue was purified by silica gel column chromatography with 4% EtOAc/hexanes to provide the desired product (1.0 g, 66% yield) as a colorless oil. ¹H NMR (400 MHz, CDCl₃) δ 5.00-4.85 (m, 1H), 3.95 (d, J=5.82 Hz, 2H), 2.63 (d, J=6.13 Hz, 2H), 2.58-2.47 (m, 2H), 2.46-2.35 (m, 4H), 2.34-2.24 (m, 4H), 1.77-1.45 (m, 15H), 1.44-1.16 (m, 36H), 0.87 (m, 12H). LRMS: 705 [M+Na]⁺.

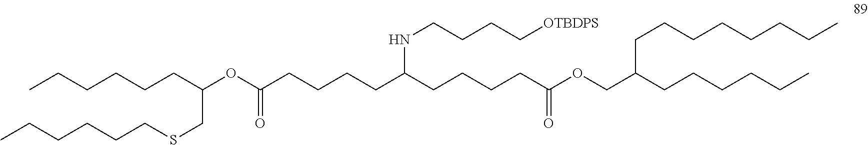

(v) 1-(2-hexyldecyl) 11-(1-(hexylthio)octan-2-yl) 6-((4-((tert-butyldiphenylsilyl)oxy)butyl)-amino)undecanedioate (89). To a solution of 88 (0.45 g, 0.66 mmol) 4-[tert-butyl(diphenyl)silyl]oxybutan-1-amine 74 (0.32 g, 0.99 mmol), and HOAc (4 mg, 0.06 mmol) in DCE (4.0 mL), sodium triacetoxyborohydride, (0.28 g, 1.32 mmol) was added portion wise and stirred at room temperature under nitrogen for 18 hours. The reaction was quenched with sat. aq. NaHCO₃ (2.0 mL), diluted with water (4.0 mL) and extracted with CH₂Cl₂ (3×5.0 mL). The combined extracts were dried (Na₂SO₄) and concentrated. The residue was purified by silica chromatography (5% MeOH in CH₂Cl₂) to yield desired product (0.49 g, 75%) as colorless oil. ¹H NMR (400 MHz, CDCl₃) δ 7.70-7.62 (m, 4H), 7.46-7.33 (m, 6H), 5.05-4.87 (m, 1H), 3.96 (d, J=5.79 Hz, 2H), 3.66 (t, J=5.88 Hz, 2H), 2.63 (d, J=6.16 Hz, 2H), 2.59-2.42 (m, 5H), 2.30 (td, J=7.55, 2.96 Hz, 4H), 1.80-1.44 (m, 17H), 1.47-1.17 (m, 42H), 1.04 (s, 9H), 0.92-0.83 (m, 12H). LRMS: 994 [M+H]⁺.

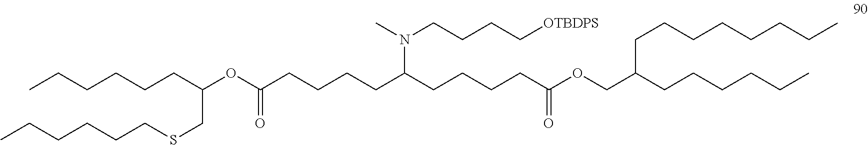

(vi) 1-(2-Hexyldecyl) 11-(1-(hexylthio)octan-2-yl) 6-((4-((tert-butyldiphenylsilyl)oxy)butyl)-(methyl)amino)undecanedioate (90). A solution of 89 (0.5 g, 0.5 mmol), sodium triacetoxyborohydride (0.21 g, 1.0 mmol), AcOH (0.05 mmol), and aq. formaldehyde (37%, 0.15 mL) was stirred in THF (2.0 mL) under nitrogen for 18 hours. The reaction was quenched with sat. aq. NaHCO₃ (2.0 mL), diluted with water (5.0 mL) and extracted with CH₂Cl₂ (3×5.0 mL). The combined organics were dried (Na₂SO₄) and concentrated. The combined extracts were dried (Na₂SO₄) and concentrated. The residue was purified by silica chromatography (4% MeOH in CH₂Cl₂) to yield desired product (0.43 g, 85%). ¹H NMR (400 MHz, CDCl₃) δ 7.70-7.58 (m, 4H), 7.49-7.31 (m, 6H), 5.02-4.85 (m, 1H), 3.96 (d, J=5.78 Hz, 2H), 3.65 (t, J=6.13 Hz, 2H), 2.63 (d, J=6.16 Hz, 2H), 2.53 (t, J=7.40 Hz, 2H), 2.37-2.24 (m, 7H), 2.12 (s, 3H), 1.75-1.12 (m, 59H), 1.04 (s, 9H), 0.90-0.83 (m, 12H). LRMS: 1008 [M+H]⁺.

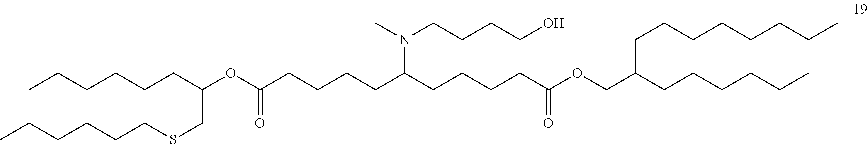 [SMILES: CCCCCCCCC(CCCCCC)COC(=O)CCCCC(CCCCC(=O)OC(CCCCCC)CSCCCCCC)N(C)CCCCO]

(vii) 1-(2-Hexyldecyl) 11-(1-(hexylthio)octan-2-yl) 6-((4-hydroxybutyl)(methyl)amino)-undecanedioate (19). To a solution of 90 (0.40 g, 0.4 mmol) in THF (1.0 mL) was added HF-pyridine (0.24 mL, 0.8 mmol) at 0° C. under inert atmosphere. The reaction was warmed to room temperature and stirred for 4 hours. Water (5.0 mL) was added, and the mixture was extracted with CH₂Cl₂ (3×5.0 mL). The combined organics were dried (Na₂SO₄) and concentrated. The residue was purified by silica chromatography (3% MeOH in CH₂Cl₂) to yield product (0.18 g, 59%). ¹H NMR (400 MHz, CDCl₃) δ 5.01-4.85 (m, 1H), 3.95 (d, J=5.74 Hz, 2H), 3.68 (t, J=7.43 Hz, 2H), 3.21-3.12 (m, 1H), 3.07 (t, J=7.43 Hz, 2H), 2.75 (s, 3H), 2.62 (d, J=6.13 Hz, 2H), 2.52 (t, J=7.40 Hz, 2H), 2.41-2.27 (m, 4H), 2.01-1.88 (m, 2H), 1.87-1.17 (m, 57H), 0.94-0.78 (m, 12H). LRMS: 770 [M+H]⁺.

### Example 2: MRNA-Containing LNPs Comprising Ionizable Lipids of the Disclosure Exhibit In Vivo Delivery of mRNA to the Liver and Spleen that is Superior to the MC3 Benchmark

LNP formulations containing 50/10/38.5/1.5 mol % of ionizable lipids 1, 5-15, 18, 19/DSPC/chol/PEG-DMG with a nitrogen-to-phosphorous ratio (N/P) of 6 and mRNA encoding luciferase were tested for in vivo transfection efficiency in the liver and spleen after injection to CD-1 mice. The mRNA dose was 1 mg/kg. Luminescence intensity in the liver or spleen was measured at 4 hours post-injection.

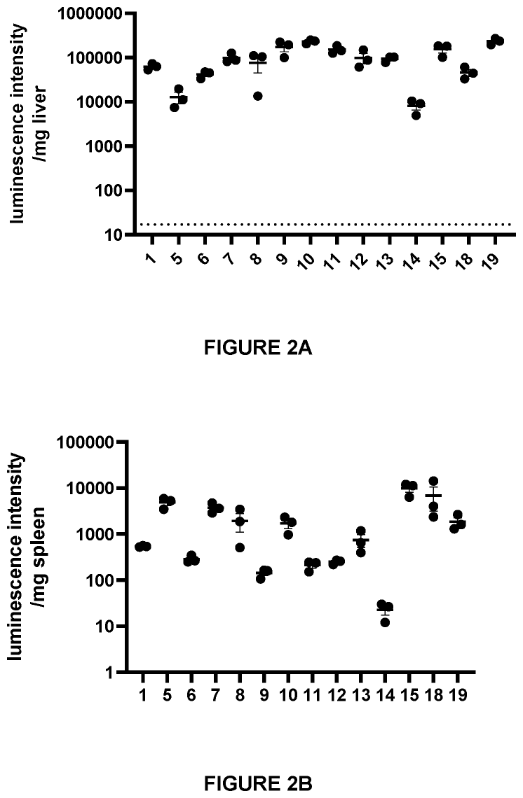

The results in FIG. 2A show that luminescence intensity per mg in the liver was higher for most of the ionizable cationic lipids tested than the MC3 benchmark, 1.

The results of FIG. 2B show that luminescence intensity per mg in the spleen was higher for many of the ionizable cationic lipids tested than the MC3 benchmark, 1.

1. A lipid having a structure of Formula A: 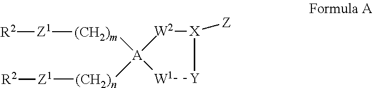
or a pharmaceutically acceptable salt thereof,
wherein
R¹ and R² are, independently, a linear or branched alkyl group comprising between six and twenty C atoms, optionally substituted, optionally comprising C═C double bonds of E or Z geometry, and optionally comprising heteroatoms selected from N, O or S, wherein
at least one of R¹ or R² comprises at least one S atom, wherein
if two or more S atoms are present in R¹ and/or R², the two or more S atoms are either bound to the same or to different respective carbon atoms, and wherein
the two or more S atoms are not bound to each other,
Z¹ and Z² are each a carboxy group 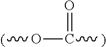
and wherein the C atom of the carboxy group is bound to the (CH₂)ₘ or (CH₂)ₙ moiety, and the O atom of the carboxy group is bound to the R¹ or R² group, or wherein the O atom of the carboxy group is bound to the (CH₂)ₘ or (CH₂)ₙ moiety, and the C atom of the carboxy group is bound to the R¹ or R² group;
m and n are, independently, 4 to 8;
A is either C or N, and if A is C, then
W¹ and Y are either bonded to each other or not bonded to each other, as indicated by the dashed bond, wherein
if W¹ and Y are bonded to each other, then
W¹ is O or S
W² is O or S
X is CH
Y is (CH₂)ₘ, wherein m is 1 or 2; and
Z is a group selected from structures a-c below, wherein the wavy line represents the bond to X:
a. 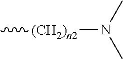
type 2 ionizable head;
b. 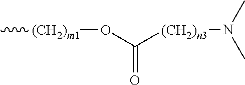
type 3 ionizable head; and
c. 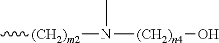
type 4 ionizable head,
if W¹ and Y are not bonded to each other, then
W¹ is H;
W² is O or S or NH or NR², wherein R² is a C₁ to C₄ alkyl optionally substituted with an OH group; and
Group 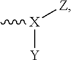
wherein the wavy line represents the bond to W², is a group selected from structures d-h below, wherein the wavy line represents the bond to W²:
d. 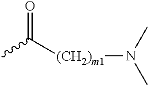
if W² is O, type 1 ionizable head;
e. 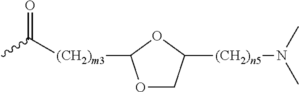
if W² is O, type 5 ionizable head;
f. 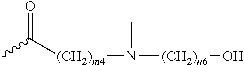
if W² is O, type 6 ionizable head;
g. [symbol] (CH₂)ₙ_{⁷}—OH if W² is NH or NR², type 7 ionizable head;
h. 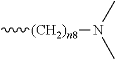
if W² is NH or NR², type 8 ionizable head; and
i. 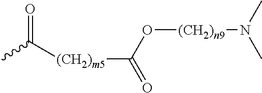
if W² is O, type 9 ionizable head,
if A is N, then
W¹ and Y are absent
W² and X together form a group of structure (CR^{a}R^{b})ₚ, wherein R^{a} and R^{b} are independently selected from H, C₁-C₅ alkyl or C₁-C₅ cycloalkyl, and wherein index p is 2 to 6; and
Z is OH or NR′R″, wherein R′ and R″ are independently C₁-C₅ alkyl, C₁-C₅ cycloalkyl, or moieties of a heterocyclic group that incorporates the N atom to which R′ and R″ are bound.

2. The lipid or the pharmaceutically acceptable salt thereof, of claim 1, wherein A is N, W¹ and Y are absent, W² and X together form a group of the structure (CR^{a}R^{b})ᵣ, and Z is NR′R″ and wherein R′ and R″ are moieties of the heterocyclic group that incorporates the N atom to which R′ and R″ are bound and wherein the heterocyclic group is selected from pyrrolidine, piperidine or morpholine.

3. The lipid or the pharmaceutically acceptable salt thereof, of claim 1, wherein A is a carbon atom.

4. The lipid or the pharmaceutically acceptable salt thereof, of claim 3, wherein W¹ and Y are not bonded to each other.

5. The lipid or the pharmaceutically acceptable salt thereof, of claim 4, wherein W² is O.

6. The lipid or the pharmaceutically acceptable salt thereof, of claim 5, wherein the moiety 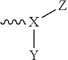
of Formula A is structure d.

7. The lipid or the pharmaceutically acceptable salt thereof, of claim 4, wherein W² is NR³ and wherein R³ is substituted with an OH group.

8. The lipid or the pharmaceutically acceptable salt thereof, of claim 7, wherein Z is structure g.

9. The lipid or the pharmaceutically acceptable salt thereof, of claim 3, wherein W¹ and Y are bonded to each other.

10. The lipid or the pharmaceutically acceptable salt thereof, of claim 9, wherein Z is structure a.

11. The lipid or the pharmaceutically acceptable salt thereof, of claim 1 having a structure of any one of the following compounds 5-19 or pharmaceutically acceptable salts thereof:

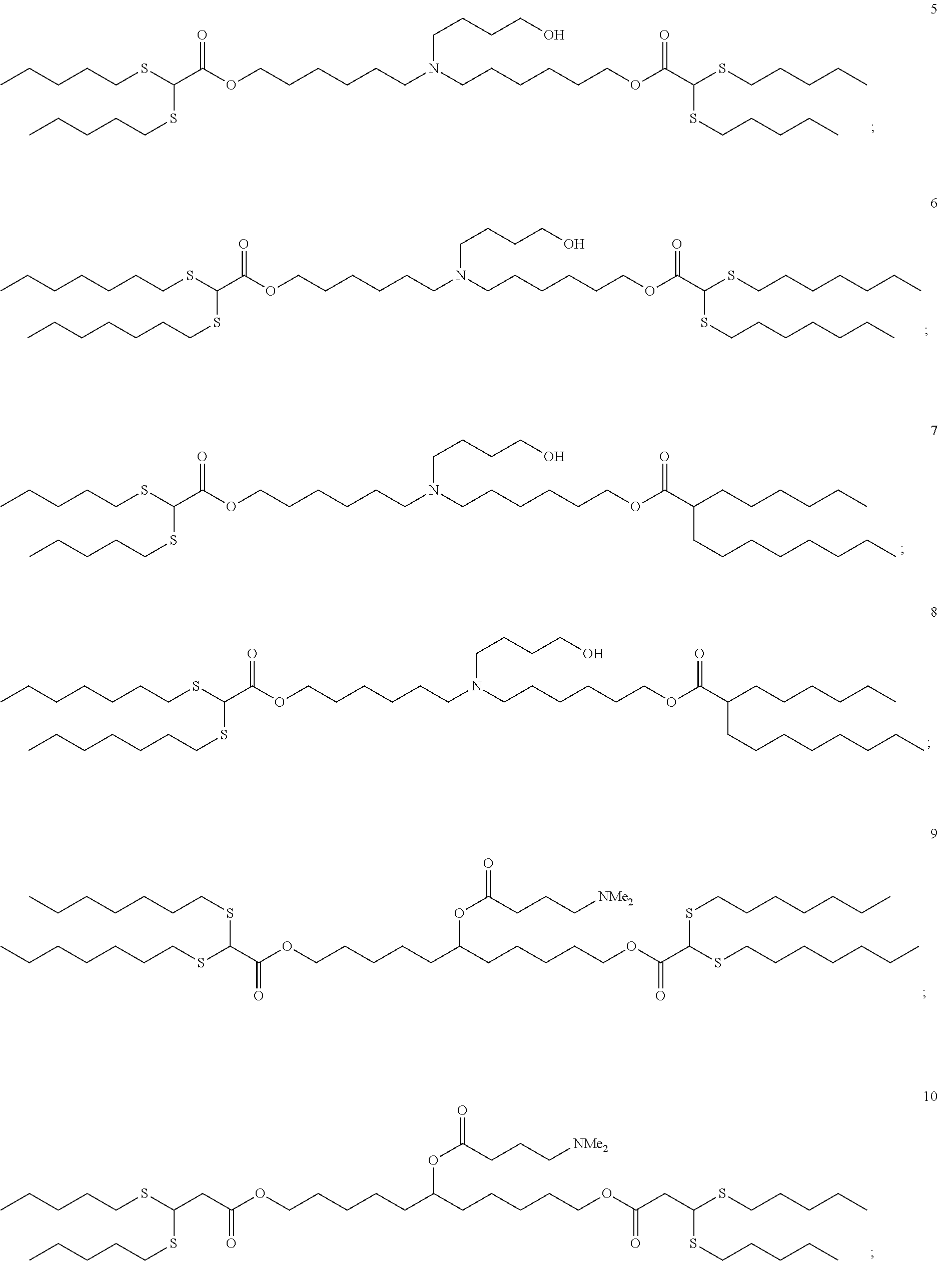

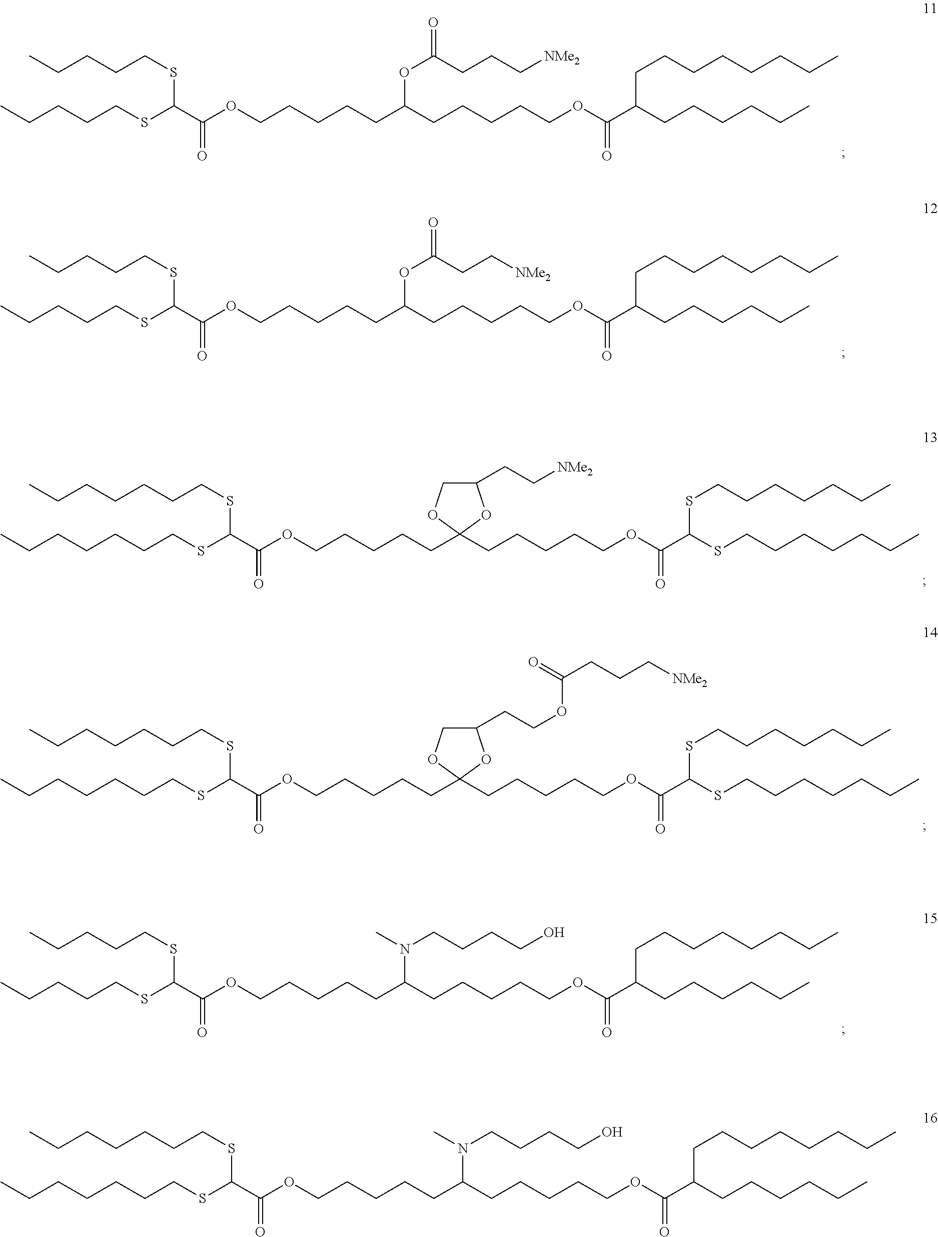

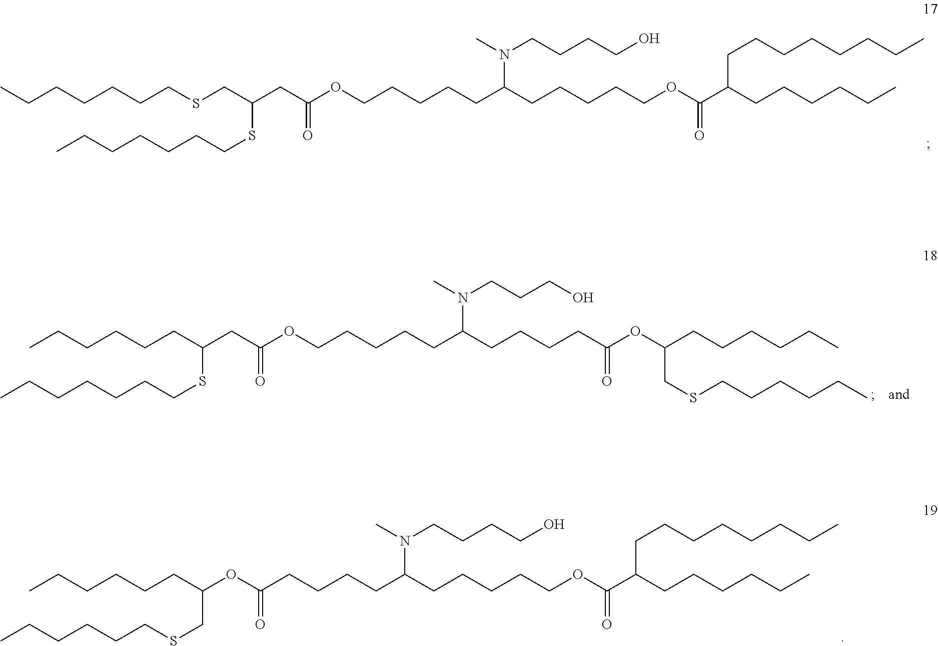

12. A lipid or a pharmaceutically acceptable salt thereof comprising:
a protonatable amino head group;
at least two lipophilic chains, wherein the protonatable amino head group has a central nitrogen atom or carbon atom to which each of the two lipophilic chains are directly bonded;
at least one of the lipophilic chains has the formula: 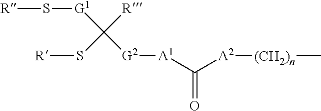
wherein R′ and R″ are, independently, linear or branched optionally substituted C₃-C₁₂ alkyl and optionally with varying degrees of unsaturation;
R′″ is H or a linear, branched, or cyclic optionally substituted C₁-C₆ alkyl group;
G¹ and G² are, independently, a group of (CR^{a}R^{b})ₚ, wherein R^{a} and R^{b} are independently selected from H or optionally substituted C₁-C₅ alkyl or cycloalkyl, and wherein p is 0 to 6;
one of A¹ and A² is an O, and a respective other one of A¹ and A² is a bond, or wherein A¹ and A² are both O;
n is 4 to 8;
each one of the lipophilic chains has between 15 and 30 carbon atoms in total; and
wherein the lipid has (i) a pKₐ of between 6 and 8; and (ii) a log P of at least 11.

13. The lipid or the pharmaceutically acceptable salt thereof, of claim 1, wherein the lipid, when formulated in a lipid nanoparticle comprising an mRNA encoding for luciferase, results in an increase in luminescence intensity per mg of organ in vivo of at least about 10% as measured in the liver and/or spleen relative to an otherwise identical lipid nanoparticle containing a DLin-MC3-DMA ionizable lipid.

14. A lipid nanoparticle comprising the lipid of claim 1 and a nucleic acid.

15. The lipid nanoparticle of claim 14, comprising a helper lipid and optionally a hydrophilic polymer-lipid conjugate.

16. The lipid nanoparticle of claim 15, wherein the helper lipid is selected from cholesterol, a diacylglycerol, a glycerophospholipid-cholesterol conjugate and a sphingolipid.

17. A lipid nanoparticle comprising:
an ionizable lipid with at least two lipophilic chains, at least one of the two lipophilic chains having a dithioacetal and/or dithioketal moiety;
one or more helper lipids;
optionally a hydrophilic polymer-lipid conjugate; and
a nucleic acid.

18. A method for administering a nucleic acid to a subject in need thereof, the method comprising preparing or providing the lipid nanoparticle of claim 14 comprising the nucleic acid and administering the lipid nanoparticle to the subject.

19. A method for delivering a cargo molecule to a cell, the method comprising contacting the lipid nanoparticle of claim 14 with the cell in vivo or in vitro.

20. The method of claim 19, wherein the cargo molecule is a nucleic acid.

21. (canceled)

22. (canceled)
